SEQUENCE LISTING (695 sequences)

SEQ ID NO: 1 - protein, 125 aa, Artificial Sequence
  Description of Artificial Sequence Synthetic polypeptide
QVQLQESGGGVVQAGGSLSLSCSPYQRASSLFAMGWFRQSPGKEREFVAGISWNGDKSQYADSVKDRFTISRDNDKNTVFLQMNSLKPEDTAVYYCAAHRASFELGFATHDYDFWGQGTQVTVSS

SEQ ID NO: 2 - protein, 111 aa, Artificial Sequence
  Description of Artificial Sequence Synthetic polypeptide
QVQLQESGGGLVQTGGSLRLSCVASGSIFSISSAVWSRQAPGKQREWVASIFSDGSTNYATSVKGRFTISRDHAKNTVYLQMNSLKPEDTGVYYCAVDGYRGQGTQVTVSS

SEQ ID NO: 3 - protein, 124 aa, Artificial Sequence
  Description of Artificial Sequence Synthetic polypeptide
QVQLQESGGGLVQAGGSLRLSCTASGRTLSYWTMGWFRQAPGKEREFVAAINWSSGTRYSDSVRDRFTIDGDTDKTTVYLEMNKMNLDDSAVYYCAAHRASFGLGYQTHEYDFWGQGTQVTVSS

SEQ ID NO: 4 - protein, 118 aa, Artificial Sequence
  Description of Artificial Sequence Synthetic polypeptide
QVQLQESGGGLVQTGDSLRLSCTASGGTFSSYTMGWYRQAPGKGREFVGSITWNSEVTYYADSVKGRFTISRDNAKNMMNLQMNSLKPEDTAVYYCAAGRAGSGFTSWGQGTQVTVSS

SEQ ID NO: 5 - protein, 131 aa, Artificial Sequence
  Description of Artificial Sequence Synthetic polypeptide
QVQLQESGGGLVQPGGSLRLSCTASGFTLDKYAVGWFRQAPGKEREGVSCISSIDDSTDYVDSVKGRFTISRDNAKNAVYLQMNSLKPEDTAVYNCMTIPLPYGSTCDIPSRSDLLAINYWGKGTLVTVSS

SEQ ID NO: 6 - protein, 126 aa, Artificial Sequence
  Description of Artificial Sequence Synthetic polypeptide
QVQLQQSGGGLVQPGGSLRLSCTASGFTVPYYYIGWFRQAPGKEREGISCIASSSGKAYYADSVKGRFTLSKDNAKNTAYLQMDSLKPEDTAVYYCAALRKYGSTCYLHVLEYDYWGQGTQVNVSS

SEQ ID NO: 7 - protein, 124 aa, Artificial Sequence
  Description of Artificial Sequence Synthetic polypeptide
QVQLQESGGGLVQAGGSLRLSCTASGRTLSYWTMGWFRQVPGKEREFVAAINWSSGTRYSESVRDRFTIDGDTDKTTVYLEMNKMNLDDSAVYYCAAHRASFGLGYQTHEYDFWGQGTQVTVSS

SEQ ID NO: 8 - protein, 124 aa, Artificial Sequence
  Description of Artificial Sequence Synthetic polypeptide
QVQLQQSGGGLVQAGGSLRLSCTASGRTLSYWTMGWFRQVPGKEREFVAAINWSSGTRYSESVRDRFTIDGDTDKTTVYLEMNKMNLDDSAVYYCAAHRASFGLGYQTHEYDFWGQGTQVTVSS

SEQ ID NO: 9 - protein, 124 aa, Artificial Sequence
  Description of Artificial Sequence Synthetic polypeptide
QVQLQQSGGGLVQAGGSLRLSCAASGSTFNNYMIGWFRQAPGKEREFVATISGSGAGTFYADSVRGRFTISRDNAKNTVYLQMNSLKLEDTAGYYCARRMSRSGIFGLRDYDSWGQGTQVTVSS

SEQ ID NO: 10 - protein, 125 aa, Artificial Sequence
  Description of Artificial Sequence Synthetic polypeptide
QVQLQQSGGGVVQAGGSLSLSCSPYQRASSLFAMGWFRQSPGKEREFVAGISWNGDKSQYADSVKDRFTISRDNDKNTVFLQMNSLKPEDTAVYYCAAHRASFELGFATHDYDFWGQGTQVTVSS

SEQ ID NO: 11 - protein, 121 aa, Artificial Sequence
  Description of Artificial Sequence Synthetic polypeptide
QVQLQESGGGLVQAGGSLRLSCAASGRTFSNADMAWFRQSPGKERESVAAISWSGGRTYYADSVKGRATISRDIAKDTVYLQMNSLKPEDTAVYYCAAGGYSNLPTSYGYWGQGTQVTVSS

SEQ ID NO: 12 - protein, 122 aa, Artificial Sequence
  Description of Artificial Sequence Synthetic polypeptide
QVQLQESGGGLVQTGGSLRLSCAASGRAFSTYGMGWFRQAPGKEREFVAGISSSGAGSAYVDSVKHRFTVSRDNAKNTMYLQMNSLKPEDTAVYYCAASTTSWGKFAHYIYWGQGTQVTVSS

SEQ ID NO: 13 - protein, 126 aa, Artificial Sequence
  Description of Artificial Sequence Synthetic polypeptide
QVQLQESGGGLVQAGGSLRLSCAASGGTFSSYIMGWFRQAPGKDREFVGAISWSGGVTHYADSVKGRFTISRDNAKNTVYLQMNSLKPEDTAVYYCAADSRISAGGSYYEADFGSWGQGTQVTVSS

SEQ ID NO: 14 - protein, 114 aa, Artificial Sequence
  Description of Artificial Sequence Synthetic polypeptide
QVQLQESGGGLVQAGGSLRLSCVVSGSIMSIRVMGWYRQAPGKQRELVATMSRGNTINYADSVRGRFTISRDNAKSTVYLQMNSLKPEDTDVYYCAALLDSYYWGQGTQVTVSS

SEQ ID NO: 15 - protein, 114 aa, Artificial Sequence
  Description of Artificial Sequence Synthetic polypeptide
QVQLQESGGGLVQAGGSLRLSCAASASIISIRVMGWYRQAPGKQRELVATMSRGGTINYADSVRGRFTISRDNAKSTVFLEMNSLKPEDTAVYYCTALLDSYYWGQGTQVTVSS

SEQ ID NO: 16 - protein, 114 aa, Artificial Sequence
  Description of Artificial Sequence Synthetic polypeptide
QVQLQESGGGLVQAGGSLRLSCAASGSIFSIRVMGWYRQAPGKQRELVATMSRGGTINYADSVRGRFTISRDNAKITVYLQMTSLKPEDTAVYYCAALLDSYYWGQGTQVTVSS

SEQ ID NO: 17 - protein, 131 aa, Artificial Sequence
  Description of Artificial Sequence Synthetic polypeptide
QVQLQESGGGLVQPGGSLRLSCTASGFTLDKYAVGWFRQAPGKEREGVSCISSIDDSTDYVDSVKGRFTISRDNAKNAVYLQMNSLKPEDTAVYDCMTIPLPYGSTCRIPSRSDLLAINYWGKGTLVTVSS

SEQ ID NO: 18 - protein, 120 aa, Artificial Sequence
  Description of Artificial Sequence Synthetic polypeptide
QVQLQESGGGLVQAGGSLRLSCQGSGRTFSTYAMGWYRQAPGKEREFVAAITRGGNTIYADSVKGRFTISRVSDKNTVYLQMSSLKPEDTAVYYCAADRIIVPRDPMDYWGKGTLVTVSS

SEQ ID NO: 19 - protein, 124 aa, Artificial Sequence
  Description of Artificial Sequence Synthetic polypeptide
QVQLQQSGGGLVQAGGSLRLSCTASGRTLSYWTMGWFRQAPGKEREFVAAINWSSGTRYSDSVRDRFTIDGDTDKTTVYLEMNKMNLDDSAVYYCAAHRASFGLGYQTHEYDFWGQGTQVTVSS

SEQ ID NO: 20 - protein, 124 aa, Artificial Sequence
  Description of Artificial Sequence Synthetic polypeptide
QVQLQESGGGLVQAGGSLRLTCTASGRTLSYWTMGWFRQAPGKEREFVAAINWSSGTRYSDSVKDRFTIDGDSDKTTVYLQMNSLNLDDSAVYYCAAHRASFGLGYQTHEYDFWGQGTQVTVSS

SEQ ID NO: 21 - protein, 126 aa, Artificial Sequence
  Description of Artificial Sequence Synthetic polypeptide
QVQLQESGGGLVQAGDSLRLSCLASGGTFSSYIMGWFRQAPGKDREFVGAISWSGGVTHYADSVKGRFTISRDNAKNTVYLQMNSLKPEDTAVYYCAADSRISAGGSYYEADFGSWGQGTQVTVSS

SEQ ID NO: 22 - protein, 122 aa, Artificial Sequence
  Description of Artificial Sequence Synthetic polypeptide
QVQLQESGGGLVQAGGSLRLTCAVSGRTFSAIHMGWFRQAPGKEREFVAGISWSGGGTAYGGTVKGRFTISRDNAKNTVSLQMNSLKSEDTAVYYCAASDTDWGRSASYDYWGQGTQVTVSA

SEQ ID NO: 23 - protein, 126 aa, Artificial Sequence
  Description of Artificial Sequence Synthetic polypeptide
QVQLQQSGGGLVQAGGSLRLSCAASGGTFSSYVMGWFRQAPGKDREFVGAISWSGGVTHYADSVKGRFTISRDNAKNTVYLQMNSLKPEDTAVYYCAADSRISAGGSYYEADFGSWGQGTQVTVSS

SEQ ID NO: 24 - protein, 120 aa, Artificial Sequence
  Description of Artificial Sequence Synthetic polypeptide
QVQLQQSGGGLVQAGDSLRLSCATSGRTFSSYTMGWFRQTPGKEREFVAAISWSGTYYTDSVKGRFTISRDTAKNTVYLQMNSLKPEDTAVYYCAVGSRRLYYSSDINYWGQGTQVTVSS

SEQ ID NO: 25 - protein, 120 aa, Artificial Sequence
  Description of Artificial Sequence Synthetic polypeptide
QVQLQESGGGLVQAGGSLRLSCATSGLTVSRYTMGWFRQTPGKDREFVAAISWSGTYYTDSVKGRFTISVDNAKNMVYLQMNSLKPEDTAVYYCAAGSRRLYYSNDINYWGQGTQVTVSS

SEQ ID NO: 26 - protein, 123 aa, Artificial Sequence
  Description of Artificial Sequence Synthetic polypeptide
QVQLQESGGGLVQAGGSLRLSCAASSRTFSNYAMAWFRQTPGKEREFLATINGDTTFTIYADSVKGRFTISRDNAKNTLYLQMNSLKAEDTAVYYCAARQWNPTMRERDYGYWGQGTEVTVSS

SEQ ID NO: 27 - protein, 126 aa, Artificial Sequence
  Description of Artificial Sequence Synthetic polypeptide
QVQLQESGGGLVQAGGSLRLSCAASGRVFENYFMGWFRQAPGKEREFVAATNWNTATNWNTYYAAFVKARFTISRDKAKNTLYLQMNSLKPEDTAVYYCAATGSRTYDVVDYYDYWGQGTQVTVSS

SEQ ID NO: 28 - protein, 124 aa, Artificial Sequence
  Description of Artificial Sequence Synthetic polypeptide
QVQLQESGGGLVQAGGSLRLSCAASGRTFSSYSMAWFRQAPGKERESVAAITYSGITTAYTDSVKGRFTIWRDNAKNTVYLQMNSLKPEDTAVYYCAASYSASRSYPFGEYDYWGQGTQVTVSS

SEQ ID NO: 29 - protein, 124 aa, Artificial Sequence
  Description of Artificial Sequence Synthetic polypeptide
QVQLQESGGGLVQAGGSLRLSCAASGRTFSSYSMAWFRQAPGKERESVAAITYSGISTAYTDSVKGRFTISRDNAKNTVYLYMNSLKPEDTAVYYCAASYSASRSYPFGEYDYWGQGTQVTVSS

SEQ ID NO: 30 - protein, 115 aa, Artificial Sequence
  Description of Artificial Sequence Synthetic polypeptide
QVQLQESGGGLVQPGGSLRLSCAASGFTFSNSAMSWMRQAPGKAVEWVSSINIGGDSRRYAESVAGRFTISRDNAKNTLYLQMNSLKPEDTAVYYCAKGLASTIRGQGTQVTVSS

SEQ ID NO: 31 - protein, 114 aa, Artificial Sequence
  Description of Artificial Sequence Synthetic polypeptide
QVQLQESGGGLVQPGGSLRLSCAASGFTFSNSAMSWMRQAPGKGVEWVSSIEVGGGRRYAESVAGRFTISRDNAKNTLYLQMNSLKPEDTAVYYCSKGLASTIRGQGTQVTVSS

SEQ ID NO: 32 - protein, 114 aa, Artificial Sequence
  Description of Artificial Sequence Synthetic polypeptide
QVQLQESGGGLVQPGGSLRLSCAASGFTFSNSAMSWMRQAPGKGVEWVSSIGIDGGRRYAEAVAGRFTISRDNAKNTLYLQMNSLKPEDTAVYYCAKGLASTIRGQGTQVTVSS

SEQ ID NO: 33 - protein, 115 aa, Artificial Sequence
  Description of Artificial Sequence Synthetic polypeptide
QVQLQESGGGLVQPGGSLRLSCAASGFTFSNSAMSWMRQAPGKGVEWVSSIGIGGGTTRYADSVAGRFTISRDNAKNTLYLQMNSLKPEDTAVYYCAKGLASTIRGQGTQVTVSS

SEQ ID NO: 34 - protein, 120 aa, Artificial Sequence
  Description of Artificial Sequence Synthetic polypeptide
QVQLQESGGGLVQAGDSLRLSCATSGRSFSSYTMGWFRQTPGKEREFVAAISWSGTYYTDSVKGRFTISRDNAKNTVYLQMNSLKPEDTAVYYCAVGSRRLYYSSDINYWGQGTQVTVSS

SEQ ID NO: 35 - protein, 120 aa, Artificial Sequence
  Description of Artificial Sequence Synthetic polypeptide
QVQLQESGGGLVQAGDSLRLSCATSGLTVSRYTMGWFRQTPGKEREFVAAISWSGTYYTDSVKGRFTISRDNAKNMVYLQMNSLKPEDTAVYYCAAGSRRLHYSSDINYWGQGTQVTVSS

SEQ ID NO: 36 - protein, 128 aa, Artificial Sequence
  Description of Artificial Sequence Synthetic polypeptide
QVQLQESGGGLVQAGESLRLSCLAAGRTFSTSTLGWFRQAPGLEREFVAAIRYTSDYTARTTDYADSVKGRFAISRDYIKQAVYLQMNNLKPEDTAVYYCAAAKYGMGYSDPSGYTYWGQGTQVTVSS

SEQ ID NO: 37 - protein, 123 aa, Artificial Sequence
  Description of Artificial Sequence Synthetic polypeptide
QVQLQESGGGLVQAGGSLRLSCAASSRTFSNYAMAWFRQTPGKEREFLAAITGDTAFTIYADSVKGRFTISRDNPKNTLYLQMNSLKAEDTAVYYCAARQWNPTMRERDYGYWGQGTEVTVSS

SEQ ID NO: 38 - protein, 127 aa, Artificial Sequence
  Description of Artificial Sequence Synthetic polypeptide
QVQLQESGGGLVQAGGSLRLSCAASGRRFRLYHMGWFRQAPGKEREFVAVISWSGGTTVYADSVKGRFTISRDNEKNAGYLQMNSLKPEDTAVYYCAVDRLIESFSDPTAWPRMDYWGKGALVTVSS

SEQ ID NO: 39 - protein, 115 aa, Artificial Sequence
  Description of Artificial Sequence Synthetic polypeptide
QVQLQESGGGLVQPGGSLRLSCAASGFTFSNSAMSWMRQAPGKGVEWVSSINIGGGTTSYADSVAGRFTISRDNAKNTLYLQMNSLKPEDTAVYYCAKGLASTIRGQGTQVTVSS

SEQ ID NO: 40 - protein, 115 aa, Artificial Sequence
  Description of Artificial Sequence Synthetic polypeptide
QVQLQESGGGLVQPGGSLRLSCAASGFTFSNSAMSWMRQAPGKGVEWVSSINIGGGTRRYAESVAGRFTISRDNAKNTLYLQMNSLKPEDTAVYYCAKGLASTIRGQGTQVTVSS

SEQ ID NO: 41 - protein, 127 aa, Artificial Sequence
  Description of Artificial Sequence Synthetic polypeptide
QVQLQESGGGLVQAGGSLRLSCAASGRKFRLYHMGWFRQAPGKEREFVAVISWSGGSTVYADSVKGRFTISRDNEKNAGYLQMNSLKPEDTAVYYCAVDRLIESFSDPTAWPRMDYWGKGALVTVSS

SEQ ID NO: 42 - protein, 114 aa, Artificial Sequence
  Description of Artificial Sequence Synthetic polypeptide
QVQLQQSGGGLVQPGGSLRLSCAASGFTFSNSAMSWMRQAPGKGVEWVSSINIGGGTRYADSVAGRFTIYRDNAKNTLYLQMNSLKSEDTAVYYCAKGLASTIRGQGTQVTVSS

SEQ ID NO: 43 - protein, 123 aa, Artificial Sequence
  Description of Artificial Sequence Synthetic polypeptide
QVQLQESGGGSVQAGGSLRLSCAASGRTFSSYDMGWFRQAPGKEREWVASISYNIYYADFVKGRFTISKDNAKNTVSLQMNSLKPEDTAVYYCAAVQRRGSYSYDRAQSYDYWGQGTQVTVSS

SEQ ID NO: 44 - protein, 114 aa, Artificial Sequence
  Description of Artificial Sequence Synthetic polypeptide
QVQLQESGGGLVQPGGSLRLSCAASGFTFSNSAMSWMRQAPGKGVEWVSSIEIGGTRRYAESVAGRFTISRDNAKNTLYLQMNSLKAEDTAVYYCAKGLASTIRGQGTQVTVSS

SEQ ID NO: 45 - protein, 115 aa, Artificial Sequence
  Description of Artificial Sequence Synthetic polypeptide
QVQLQESGGGLVQPGGSLRLSCAASGFTFSNSPMSWMRQAPGKGVEWVSSINIGAGTTRYAESVAGRFTIARDNAKNTLYLQMNSLKPEDTAVYYCAKGLASTIRGQGTQVIVSS

SEQ ID NO: 46 - protein, 115 aa, Artificial Sequence
  Description of Artificial Sequence Synthetic polypeptide
QVQLQESGGGLVQPGGSLRLSCAASGFTFSNSAMSWMRQAPGKGVEWVSSINIGGGDKRYAESVAGRFTISRDNAKNTLYLQMNSLKFEDTAVYYCAKGLASTIRGQGTQVTVSS

SEQ ID NO: 47 - protein, 114 aa, Artificial Sequence
  Description of Artificial Sequence Synthetic polypeptide
QVQLQESGGGLVQPGGSLRLSCAASGFTFSNSAMSWMRQAPGKGVEWVSSIETGGTKRYAESVAGRFTISRDNAKNTLNLQMNSLKPEDTAVYYCAKGLASTIRGQGTQVTVSS

SEQ ID NO: 48 - protein, 115 aa, Artificial Sequence
  Description of Artificial Sequence Synthetic polypeptide
QVQLQQSGGGLVQPGGSLRLSCAASGFTFSNSPMSWMRQAPGKGVEWVSSINIGEGTTRYAESVAGRFTISRDNVKNTLYLQMNSLKPEDTAVYYCAKGLASTIRGQGTQVTVSS

SEQ ID NO: 49 - protein, 115 aa, Artificial Sequence
  Description of Artificial Sequence Synthetic polypeptide
QVQLQESGGGLVQPGGSLRLSCAASGFTFSNSPMSWMRQAPGKGVEWVSSINIGGDTRRYAESVAGRFTISRDNAKNTLYLQMNSLKSEDTAVYYCAKGLASTIRGQGTQVTVSS

SEQ ID NO: 50 - protein, 114 aa, Artificial Sequence
  Description of Artificial Sequence Synthetic polypeptide
QVQLQESGGGLVQPGGSLRLSCAASGFTFSNSAMSWMRQAPGNGVEWVSSVNIDGGRRYAEAVAGRFTISRDNAKNTLYLQMNSLKPEDTAVYYCAKGLASTIRGQGTQVTVSS

SEQ ID NO: 51 - protein, 114 aa, Artificial Sequence
  Description of Artificial Sequence Synthetic polypeptide
QVQLQESGGGLVQPGGSLRLSCAASGFTFSNSAMAWMRQAPGKGVEWVSSISIDGGRRYAEAVAGRFTISRDNAKNTLYLQMNSLKPEDTAVYYCAKGLASTIRGQGTQVTVSS

SEQ ID NO: 52 - protein, 127 aa, Artificial Sequence
  Description of Artificial Sequence Synthetic polypeptide
QVQLQESGGGLVQAGGSLRLSCAASGGKFTLYHMGWFRQTPGKEREFVAVISWSGRSTVYADSVKGRFTISRDNDKNAGYLQMNSLKPEDTAIYYCAVDRLIEKFSDPTAWPRMDSWGRGTLVTVSS

SEQ ID NO: 53 - protein, 122 aa, Artificial Sequence
  Description of Artificial Sequence Synthetic polypeptide
QVQLQESGGGLVQAGDSLRLSCAASGRTASMGWFRQAPGTQREFVATITRSSIYTDYSDSVKGRFAISRDNAKNTVYLQMNSLKPEDTAVYYCAADSTMSGSSRYSSDYAYWGQGTQVTVSS

SEQ ID NO: 54 - protein, 114 aa, Artificial Sequence
  Description of Artificial Sequence Synthetic polypeptide
QVQLQESGGGLVQPGGSLRLSCAASGFTFSNSPMSWMRQAPGKGVEWVSSIDIGGNRRYAEAVAGRFTISRDNAKNTLYLQMNSLKPEDTAVYYCAKGLASTIRGQGTQVTVSS

SEQ ID NO: 55 - protein, 127 aa, Artificial Sequence
  Description of Artificial Sequence Synthetic polypeptide
QVQLQESGGGLVQAGGSLRLSCAVSGRRFTLYHMGWFRQRPGKEREFVAVISWSGGSTVYADSVKGRFTISRDNEKNAGYLQMNSLKPEDTAVYYCAVDRLIESFSDPTAWPRMDYWGKGALVTVSS

SEQ ID NO: 56 - protein, 127 aa, Artificial Sequence
  Description of Artificial Sequence Synthetic polypeptide
QVQLQQSGGGLVQAGGSLRLSCAASGRRFSLYHMGWFRQAPGKEREFVAVISWSGGTTVYADSVKGRFTISRDNEKNAGYLQMNSLKPEDTAVYYCAVDRLIESFSDPTAWPRMDYWGKGALVTVSS

SEQ ID NO: 57 - protein, 9 aa, Artificial Sequence
  Description of Artificial Sequence Synthetic peptide
QRASSLFAM

SEQ ID NO: 58 - protein, 9 aa, Artificial Sequence
  Description of Artificial Sequence Synthetic peptide
GSIFSISSA

SEQ ID NO: 59 - protein, 9 aa, Artificial Sequence
  Description of Artificial Sequence Synthetic peptide
GRTLSYWTM

SEQ ID NO: 60 - protein, 9 aa, Artificial Sequence
  Description of Artificial Sequence Synthetic peptide
GGTFSSYTM

SEQ ID NO: 61 - protein, 9 aa, Artificial Sequence
  Description of Artificial Sequence Synthetic peptide
GFTLDKYAV

SEQ ID NO: 62 - protein, 9 aa, Artificial Sequence
  Description of Artificial Sequence Synthetic peptide
GFTVPYYYI

SEQ ID NO: 63 - protein, 9 aa, Artificial Sequence
  Description of Artificial Sequence Synthetic peptide
GSTFNNYMI

SEQ ID NO: 64 - protein, 9 aa, Artificial Sequence
  Description of Artificial Sequence Synthetic peptide
GRTFSNADM

SEQ ID NO: 65 - protein, 9 aa, Artificial Sequence
  Description of Artificial Sequence Synthetic peptide
GRAFSTYGM

SEQ ID NO: 66 - protein, 9 aa, Artificial Sequence
  Description of Artificial Sequence Synthetic peptide
GGTFSSYIM

SEQ ID NO: 67 - protein, 9 aa, Artificial Sequence
  Description of Artificial Sequence Synthetic peptide
GSIMSIRVM

SEQ ID NO: 68 - protein, 9 aa, Artificial Sequence
  Description of Artificial Sequence Synthetic peptide
ASIISIRVM

SEQ ID NO: 69 - protein, 9 aa, Artificial Sequence
  Description of Artificial Sequence Synthetic peptide
GSIFSIRVM

SEQ ID NO: 70 - protein, 9 aa, Artificial Sequence
  Description of Artificial Sequence Synthetic peptide
GFTLDKYAV

SEQ ID NO: 71 - protein, 9 aa, Artificial Sequence
  Description of Artificial Sequence Synthetic peptide
GRTFSTYAM

SEQ ID NO: 72 - protein, 9 aa, Artificial Sequence
  Description of Artificial Sequence Synthetic peptide
GRTFSAIHM

SEQ ID NO: 73 - protein, 9 aa, Artificial Sequence
  Description of Artificial Sequence Synthetic peptide
GGTFSSYVM

SEQ ID NO: 74 - protein, 9 aa, Artificial Sequence
  Description of Artificial Sequence Synthetic peptide
GRTFSSYTM

SEQ ID NO: 75 - protein, 9 aa, Artificial Sequence
  Description of Artificial Sequence Synthetic peptide
GLTVSRYTM

SEQ ID NO: 76 - protein, 9 aa, Artificial Sequence
  Description of Artificial Sequence Synthetic peptide
SRTFSNYAM

SEQ ID NO: 77 - protein, 9 aa, Artificial Sequence
  Description of Artificial Sequence Synthetic peptide
GRVFENYFM

SEQ ID NO: 78 - protein, 9 aa, Artificial Sequence
  Description of Artificial Sequence Synthetic peptide
GRTFSSYSM

SEQ ID NO: 79 - protein, 9 aa, Artificial Sequence
  Description of Artificial Sequence Synthetic peptide
GRTFSSYSM

SEQ ID NO: 80 - protein, 9 aa, Artificial Sequence
  Description of Artificial Sequence Synthetic peptide
GFTFSNSAM

SEQ ID NO: 81 - protein, 9 aa, Artificial Sequence
  Description of Artificial Sequence Synthetic peptide
GFTFSNSAM

SEQ ID NO: 82 - protein, 9 aa, Artificial Sequence
  Description of Artificial Sequence Synthetic peptide
GFTFSNSAM

SEQ ID NO: 83 - protein, 9 aa, Artificial Sequence
  Description of Artificial Sequence Synthetic peptide
GFTFSNSAM

SEQ ID NO: 84 - protein, 9 aa, Artificial Sequence
  Description of Artificial Sequence Synthetic peptide
GRSFSSYTM

SEQ ID NO: 85 - protein, 9 aa, Artificial Sequence
  Description of Artificial Sequence Synthetic peptide
GLTVSRYTM

SEQ ID NO: 86 - protein, 9 aa, Artificial Sequence
  Description of Artificial Sequence Synthetic peptide
GRTFSTSTL

SEQ ID NO: 87 - protein, 9 aa, Artificial Sequence
  Description of Artificial Sequence Synthetic peptide
SRTFSNYAM

SEQ ID NO: 88 - protein, 9 aa, Artificial Sequence
  Description of Artificial Sequence Synthetic peptide
GRRFRLYHM

SEQ ID NO: 89 - protein, 9 aa, Artificial Sequence
  Description of Artificial Sequence Synthetic peptide
GFTFSNSAM

SEQ ID NO: 90 - protein, 9 aa, Artificial Sequence
  Description of Artificial Sequence Synthetic peptide
GFTFSNSAM

SEQ ID NO: 91 - protein, 9 aa, Artificial Sequence
  Description of Artificial Sequence Synthetic peptide
GRKFRLYHM

SEQ ID NO: 92 - protein, 9 aa, Artificial Sequence
  Description of Artificial Sequence Synthetic peptide
GFTFSNSAM

SEQ ID NO: 93 - protein, 9 aa, Artificial Sequence
  Description of Artificial Sequence Synthetic peptide
GRTFSSYDM

SEQ ID NO: 94 - protein, 9 aa, Artificial Sequence
  Description of Artificial Sequence Synthetic peptide
GFTFSNSAM

SEQ ID NO: 95 - protein, 9 aa, Artificial Sequence
  Description of Artificial Sequence Synthetic peptide
GFTFSNSPM

SEQ ID NO: 96 - protein, 9 aa, Artificial Sequence
  Description of Artificial Sequence Synthetic peptide
GFTFSNSAM

SEQ ID NO: 97 - protein, 9 aa, Artificial Sequence
  Description of Artificial Sequence Synthetic peptide
GFTFSNSAM

SEQ ID NO: 98 - protein, 9 aa, Artificial Sequence
  Description of Artificial Sequence Synthetic peptide
GFTFSNSPM

SEQ ID NO: 99 - protein, 9 aa, Artificial Sequence
  Description of Artificial Sequence Synthetic peptide
GFTFSNSPM

SEQ ID NO: 100 - protein, 9 aa, Artificial Sequence
  Description of Artificial Sequence Synthetic peptide
GFTFSNSAM

SEQ ID NO: 101 - protein, 9 aa, Artificial Sequence
  Description of Artificial Sequence Synthetic peptide
GFTFSNSAM

SEQ ID NO: 102 - protein, 9 aa, Artificial Sequence
  Description of Artificial Sequence Synthetic peptide
GGKFTLYHM

SEQ ID NO: 103 - protein, 6 aa, Artificial Sequence
  Description of Artificial Sequence Synthetic peptide
GRTASM

SEQ ID NO: 104 - protein, 9 aa, Artificial Sequence
  Description of Artificial Sequence Synthetic peptide
GFTFSNSPM

SEQ ID NO: 105 - protein, 9 aa, Artificial Sequence
  Description of Artificial Sequence Synthetic peptide
GRRFTLYHM

SEQ ID NO: 106 - protein, 9 aa, Artificial Sequence
  Description of Artificial Sequence Synthetic peptide
GRRFSLYHM

SEQ ID NO: 107 - protein, 8 aa, Artificial Sequence
  Description of Artificial Sequence Synthetic peptide
ISWNGDKS

SEQ ID NO: 108 - protein, 7 aa, Artificial Sequence
  Description of Artificial Sequence Synthetic peptide
IFSDGST

SEQ ID NO: 109 - protein, 7 aa, Artificial Sequence
  Description of Artificial Sequence Synthetic peptide
INWSSGT

SEQ ID NO: 110 - protein, 8 aa, Artificial Sequence
  Description of Artificial Sequence Synthetic peptide
ITWNSEVT

SEQ ID NO: 111 - protein, 8 aa, Artificial Sequence
  Description of Artificial Sequence Synthetic peptide
ISSIDDST

SEQ ID NO: 112 - protein, 8 aa, Artificial Sequence
  Description of Artificial Sequence Synthetic peptide
IASSSGKA

SEQ ID NO: 113 - protein, 8 aa, Artificial Sequence
  Description of Artificial Sequence Synthetic peptide
ISGSGAGT

SEQ ID NO: 114 - protein, 8 aa, Artificial Sequence
  Description of Artificial Sequence Synthetic peptide
ISWSGGRT

SEQ ID NO: 115 - protein, 8 aa, Artificial Sequence
  Description of Artificial Sequence Synthetic peptide
ISSSGAGS

SEQ ID NO: 116 - protein, 8 aa, Artificial Sequence
  Description of Artificial Sequence Synthetic peptide
ISWSGGVT

SEQ ID NO: 117 - protein, 7 aa, Artificial Sequence
  Description of Artificial Sequence Synthetic peptide
MSRGNTI

SEQ ID NO: 118 - protein, 7 aa, Artificial Sequence
  Description of Artificial Sequence Synthetic peptide
MSRGGTI

SEQ ID NO: 119 - protein, 7 aa, Artificial Sequence
  Description of Artificial Sequence Synthetic peptide
MSRGGTI

SEQ ID NO: 120 - protein, 8 aa, Artificial Sequence
  Description of Artificial Sequence Synthetic peptide
ISSIDDST

SEQ ID NO: 121 - protein, 7 aa, Artificial Sequence
  Description of Artificial Sequence Synthetic peptide
ITRGGNT

SEQ ID NO: 122 - protein, 8 aa, Artificial Sequence
  Description of Artificial Sequence Synthetic peptide
ISWSGGGT

SEQ ID NO: 123 - protein, 8 aa, Artificial Sequence
  Description of Artificial Sequence Synthetic peptide
ISWSGGVT

SEQ ID NO: 124 - protein, 6 aa, Artificial Sequence
  Description of Artificial Sequence Synthetic peptide
ISWSGT

SEQ ID NO: 125 - protein, 6 aa, Artificial Sequence
  Description of Artificial Sequence Synthetic peptide
ISWSGT

SEQ ID NO: 126 - protein, 8 aa, Artificial Sequence
  Description of Artificial Sequence Synthetic peptide
INGDTTFT

SEQ ID NO: 127 - protein, 11 aa, Artificial Sequence
  Description of Artificial Sequence Synthetic peptide
TNWNTATNWNT

SEQ ID NO: 128 - protein, 8 aa, Artificial Sequence
  Description of Artificial Sequence Synthetic peptide
ITYSGITT

SEQ ID NO: 129 - protein, 8 aa, Artificial Sequence
  Description of Artificial Sequence Synthetic peptide
ITYSGIST

SEQ ID NO: 130 - protein, 8 aa, Artificial Sequence
  Description of Artificial Sequence Synthetic peptide
INIGGDSR

SEQ ID NO: 131 - protein, 7 aa, Artificial Sequence
  Description of Artificial Sequence Synthetic peptide
IEVGGGR

SEQ ID NO: 132 - protein, 7 aa, Artificial Sequence
  Description of Artificial Sequence Synthetic peptide
IGIDGGR

SEQ ID NO: 133 - protein, 8 aa, Artificial Sequence
  Description of Artificial Sequence Synthetic peptide
IGIGGGTT

SEQ ID NO: 134 - protein, 6 aa, Artificial Sequence
  Description of Artificial Sequence Synthetic peptide
ISWSGT

SEQ ID NO: 135 - protein, 6 aa, Artificial Sequence
  Description of Artificial Sequence Synthetic peptide
ISWSGT

SEQ ID NO: 136 - protein, 12 aa, Artificial Sequence
  Description of Artificial Sequence Synthetic peptide
IRYTSDYTARTT

SEQ ID NO: 137 - protein, 8 aa, Artificial Sequence
  Description of Artificial Sequence Synthetic peptide
ITGDTAFT

SEQ ID NO: 138 - protein, 8 aa, Artificial Sequence
  Description of Artificial Sequence Synthetic peptide
ISWSGGTT

SEQ ID NO: 139 - protein, 8 aa, Artificial Sequence
  Description of Artificial Sequence Synthetic peptide
INIGGGTT

SEQ ID NO: 140 - protein, 8 aa, Artificial Sequence
  Description of Artificial Sequence Synthetic peptide
INIGGGTR

SEQ ID NO: 141 - protein, 8 aa, Artificial Sequence
  Description of Artificial Sequence Synthetic peptide
ISWSGGST

SEQ ID NO: 142 - protein, 7 aa, Artificial Sequence
  Description of Artificial Sequence Synthetic peptide
INIGGGT

SEQ ID NO: 143 - protein, 5 aa, Artificial Sequence
  Description of Artificial Sequence Synthetic peptide
ISYNI

SEQ ID NO: 144 - protein, 7 aa, Artificial Sequence
  Description of Artificial Sequence Synthetic peptide
IEIGGTR

SEQ ID NO: 145 - protein, 8 aa, Artificial Sequence
  Description of Artificial Sequence Synthetic peptide
INIGAGTT

SEQ ID NO: 146 - protein, 8 aa, Artificial Sequence
  Description of Artificial Sequence Synthetic peptide
INIGGGDK

SEQ ID NO: 147 - protein, 7 aa, Artificial Sequence
  Description of Artificial Sequence Synthetic peptide
IETGGTK

SEQ ID NO: 148 - protein, 8 aa, Artificial Sequence
  Description of Artificial Sequence Synthetic peptide
INIGEGTT

SEQ ID NO: 149 - protein, 8 aa, Artificial Sequence
  Description of Artificial Sequence Synthetic peptide
INIGGDTR

SEQ ID NO: 150 - protein, 7 aa, Artificial Sequence
  Description of Artificial Sequence Synthetic peptide
VNIDGGR

SEQ ID NO: 151 - protein, 7 aa, Artificial Sequence
  Description of Artificial Sequence Synthetic peptide
ISIDGGR

SEQ ID NO: 152 - protein, 8 aa, Artificial Sequence
  Description of Artificial Sequence Synthetic peptide
ISWSGRST

SEQ ID NO: 153 - protein, 8 aa, Artificial Sequence
  Description of Artificial Sequence Synthetic peptide
ITRSSIYT

SEQ ID NO: 154 - protein, 7 aa, Artificial Sequence
  Description of Artificial Sequence Synthetic peptide
IDIGGNR

SEQ ID NO: 155 - protein, 8 aa, Artificial Sequence
  Description of Artificial Sequence Synthetic peptide
ISWSGGST

SEQ ID NO: 156 - protein, 8 aa, Artificial Sequence
  Description of Artificial Sequence Synthetic peptide
ISWSGGTT

SEQ ID NO: 157 - protein, 18 aa, Artificial Sequence
  Description of Artificial Sequence Synthetic peptide
AAHRASFELGFATHDYDF

SEQ ID NO: 158 - protein, 5 aa, Artificial Sequence
  Description of Artificial Sequence Synthetic peptide
AVDGY

SEQ ID NO: 159 - protein, 18 aa, Artificial Sequence
  Description of Artificial Sequence Synthetic peptide
AAHRASFGLGYQTHEYDF

SEQ ID NO: 160 - protein, 11 aa, Artificial Sequence
  Description of Artificial Sequence Synthetic peptide
AAGRAGSGFTS

SEQ ID NO: 161 - protein, 24 aa, Artificial Sequence
  Description of Artificial Sequence Synthetic peptide
MTIPLPYGSTCDIPSRSDLLAINY

SEQ ID NO: 162 - protein, 19 aa, Artificial Sequence
  Description of Artificial Sequence Synthetic peptide
AALRKYGSTCYLHVLEYDY

SEQ ID NO: 163 - protein, 17 aa, Artificial Sequence
  Description of Artificial Sequence Synthetic peptide
ARRMSRSGIFGLRDYDS

SEQ ID NO: 164 - protein, 14 aa, Artificial Sequence
  Description of Artificial Sequence Synthetic peptide
AAGGYSNLPTSYGY

SEQ ID NO: 165 - protein, 15 aa, Artificial Sequence
  Description of Artificial Sequence Synthetic peptide
AASTTSWGKFAHYIY

SEQ ID NO: 166 - protein, 19 aa, Artificial Sequence
  Description of Artificial Sequence Synthetic peptide
AADSRISAGGSYYEADFGS

SEQ ID NO: 167 - protein, 8 aa, Artificial Sequence
  Description of Artificial Sequence Synthetic peptide
AALLDSYY

SEQ ID NO: 168 - protein, 8 aa, Artificial Sequence
  Description of Artificial Sequence Synthetic peptide
TALLDSYY

SEQ ID NO: 169 - protein, 8 aa, Artificial Sequence
  Description of Artificial Sequence Synthetic peptide
AALLDSYY

SEQ ID NO: 170 - protein, 24 aa, Artificial Sequence
  Description of Artificial Sequence Synthetic peptide
MTIPLPYGSTCRIPSRSDLLAINY

SEQ ID NO: 171 - protein, 14 aa, Artificial Sequence
  Description of Artificial Sequence Synthetic peptide
AADRIIVPRDPMDY

SEQ ID NO: 172 - protein, 15 aa, Artificial Sequence
  Description of Artificial Sequence Synthetic peptide
AASDTDWGRSASYDY

SEQ ID NO: 173 - protein, 19 aa, Artificial Sequence
  Description of Artificial Sequence Synthetic peptide
AADSRISAGGSYYEADFGS

SEQ ID NO: 174 - protein, 15 aa, Artificial Sequence
  Description of Artificial Sequence Synthetic peptide
AVGSRRLYYSSDINY

SEQ ID NO: 175 - protein, 15 aa, Artificial Sequence
  Description of Artificial Sequence Synthetic peptide
AAGSRRLYYSNDINY

SEQ ID NO: 176 - protein, 16 aa, Artificial Sequence
  Description of Artificial Sequence Synthetic peptide
AARQWNPTMRERDYGY

SEQ ID NO: 177 - protein, 16 aa, Artificial Sequence
  Description of Artificial Sequence Synthetic peptide
AATGSRTYDVVDYYDY

SEQ ID NO: 178 - protein, 17 aa, Artificial Sequence
  Description of Artificial Sequence Synthetic peptide
AASYSASRSYPFGEYDY

SEQ ID NO: 179 - protein, 17 aa, Artificial Sequence
  Description of Artificial Sequence Synthetic peptide
AASYSASRSYPFGEYDY

SEQ ID NO: 180 - protein, 8 aa, Artificial Sequence
  Description of Artificial Sequence Synthetic peptide
AKGLASTI

SEQ ID NO: 181 - protein, 8 aa, Artificial Sequence
  Description of Artificial Sequence Synthetic peptide
SKGLASTI

SEQ ID NO: 182 - protein, 8 aa, Artificial Sequence
  Description of Artificial Sequence Synthetic peptide
AKGLASTI

SEQ ID NO: 183 - protein, 8 aa, Artificial Sequence
  Description of Artificial Sequence Synthetic peptide
AKGLASTI

SEQ ID NO: 184 - protein, 15 aa, Artificial Sequence
  Description of Artificial Sequence Synthetic peptide
AVGSRRLYYSSDINY

SEQ ID NO: 185 - protein, 15 aa, Artificial Sequence
  Description of Artificial Sequence Synthetic peptide
AAGSRRLHYSSDINY

SEQ ID NO: 186 - protein, 17 aa, Artificial Sequence
  Description of Artificial Sequence Synthetic peptide
AAAKYGMGYSDPSGYTY

SEQ ID NO: 187 - protein, 16 aa, Artificial Sequence
  Description of Artificial Sequence Synthetic peptide
AARQWNPTMRERDYGY

SEQ ID NO: 188 - protein, 18 aa, Artificial Sequence
  Description of Artificial Sequence Synthetic peptide
AVDRLIESFSDPTAWPRM

SEQ ID NO: 189 - protein, 8 aa, Artificial Sequence
  Description of Artificial Sequence Synthetic peptide
AKGLASTI

SEQ ID NO: 190 - protein, 8 aa, Artificial Sequence
  Description of Artificial Sequence Synthetic peptide
AKGLASTI

SEQ ID NO: 191 - protein, 18 aa, Artificial Sequence
  Description of Artificial Sequence Synthetic peptide
AVDRLIESFSDPTAWPRM

SEQ ID NO: 192 - protein, 8 aa, Artificial Sequence
  Description of Artificial Sequence Synthetic peptide
AKGLASTI

SEQ ID NO: 193 - protein, 19 aa, Artificial Sequence
  Description of Artificial Sequence Synthetic peptide
AAVQRRGSYSYDRAQSYDY

SEQ ID NO: 194 - protein, 8 aa, Artificial Sequence
  Description of Artificial Sequence Synthetic peptide
AKGLASTI

SEQ ID NO: 195 - protein, 8 aa, Artificial Sequence
  Description of Artificial Sequence Synthetic peptide
AKGLASTI

SEQ ID NO: 196 - protein, 8 aa, Artificial Sequence
  Description of Artificial Sequence Synthetic peptide
AKGLASTI

SEQ ID NO: 197 - protein, 8 aa, Artificial Sequence
  Description of Artificial Sequence Synthetic peptide
AKGLASTI

SEQ ID NO: 198 - protein, 8 aa, Artificial Sequence
  Description of Artificial Sequence Synthetic peptide
AKGLASTI

SEQ ID NO: 199 - protein, 8 aa, Artificial Sequence
  Description of Artificial Sequence Synthetic peptide
AKGLASTI

SEQ ID NO: 200 - protein, 8 aa, Artificial Sequence
  Description of Artificial Sequence Synthetic peptide
AKGLASTI

SEQ ID NO: 201 - protein, 8 aa, Artificial Sequence
  Description of Artificial Sequence Synthetic peptide
AKGLASTI

SEQ ID NO: 202 - protein, 20 aa, Artificial Sequence
  Description of Artificial Sequence Synthetic peptide
AVDRLIEKFSDPTAWPRMDS

SEQ ID NO: 203 - protein, 18 aa, Artificial Sequence
  Description of Artificial Sequence Synthetic peptide
AADSTMSGSSRYSSDYAY

SEQ ID NO: 204 - protein, 8 aa, Artificial Sequence
  Description of Artificial Sequence Synthetic peptide
AKGLASTI

SEQ ID NO: 205 - protein, 20 aa, Artificial Sequence
  Description of Artificial Sequence Synthetic peptide
AVDRLIESFSDPTAWPRMDY

SEQ ID NO: 206 - protein, 20 aa, Artificial Sequence
  Description of Artificial Sequence Synthetic peptide
AVDRLIESFSDPTAWPRMDY

SEQ ID NO: 207 - protein, 322 aa, Clostridium perfringens
MKRLKIISITLVLTSVISTSLFSTQTQVFASELNDINKIELKNLSGEIIKENGKEAIKYTSSDTASHKGWKATLSGTFIEDPHSDKKTALLNLEGFIPSDKQIFGSKYYGKMKWPETYRINVKSADVNNNIKIANSIPKNTIDKKDVSNSIGYSIGGNISVEGKTAGAGINASYNVQNTISYEQPDFRTIQRKDDANLASWDIKFVETKDGYNIDSYHAIYGNQLFMKSRLYNNGDKNFTDDRDLSTLISGGFSPNMALALTAPKNAKESVIIVEYQRFDNDYILNWETTQWRGTNKLSSTSEYNEFMFKINWQDHKIEYYL

SEQ ID NO: 208 - protein, 398 aa, Clostridium perfringens
MKRKICKALICAALATSLWAGASTKVYAWDGKIDGTGTHAMIVTQGVSILENDLSKNEPESVRKNLEILKENMHELQLGSTYPDYDKNAYDLYQDHFWDPDTDNNFSKDNSWYLAYSIPDTGESQIRKFSALARYEWQRGNYKQATFYLGEAMHYFGDIDTPYHPANVTAVDSAGHVKFETFAEERKEQYKINTAGCKTNEDFYADILKNKDFNAWSKEYARGFAKTGKSIYYSHASMSHSWDDWDYAAKVTLANSQKGTAGYIYRFLHDVSEGNDPSVGKNVKELVAYISTSGEKDAGTDDYMYFGIKTKDGKTQEWEMDNPGNDFMTGSKDTYTFKLKDENLKIDDIQNMWIRKRKYTAFPDAYKPENIKIIANGKVVVDKDINEWISGNSTYNIK

SEQ ID NO: 209 - protein, 265 aa, Clostridium perfringens
MKKLIVKSTMMLLFSCLLCLGIQLPNTVKANEVNKYQSVMVQYLEAFKNYDIDTIVDISKDSRTVTKEEYKNMLMEFKYDPNQKLKSYEITGSRKIDNGEIFSVKTEFLNGAIYNMEFTVSYIDNKLMVSNMNRISIVNEGKCIPTPSFRTQVCTWDDELSQYIGDAVSFTRSSKFQYSSNTITLNFRQYATSGSRSLKVKYSVVDHWMWGDDIRASQWVYGENPDYARQIKLYLGSGETFKNYRIKVENYTPASIKVFGEGYCY

SEQ ID NO: 210 - protein, 696 aa, Clostridium perfringens
MKINKKIFSMLFMVIVLFTCISSNFSVSASSIQRGRDISNEVVTSLVATPNSINDGGNVQVRLEFKENHQRNIQSGDTITVKWTNSGEVFFEGYEKTIPLYIKDQNVGQAVIEKTGATLTFNDKIDKLDDVGGWATFTLQGRNITSGNHEHTGIAYIISGSKRADVNITKPESGTTSVFYYKTGSMYTNDTNHVNWWLLVNPSKVYSEKNVYIQDEIQGGQTLEPDSFEIVVTWYDGYVEKFKGKEAIREFHNKYPNSNISVSENKITVNISQEDSTQKFINIFYKTKITNPKQKEFVNNTKAWFKEYNKPAVNGESFNHSVQNINADAGVNGTVKGELKIIKTLKDKSIPIKDVQFKMRRVDNTVIKDGKKELLLTTDDKGIANVKGLPVGKYEVKEISAPEWIAFNPLIAPKLEFTISDQDTEGKLWAVENELKTISIPVEKVWVGQTSERAEIKLFADGIEVDKVILNADNNWKHTFENKPEYNSETKQKINYSVSETTISGYESNITGDAKNGFIVTNTELPDLTIGKEVIGELGDKTKVFNFELTLKQADGKPINGKFNYIGSVDDRYKKESIKPSDGEITFIEGKATITLSHGQEITIKDLPYGVTYKVMEKEANENGYLTTYNGNNEVTTGELKQDTKVQVVNNKEFVPTTGISTTTEQGTMVGMVIFSIGILMVMIVVLLQLNKGLKR

SEQ ID NO: 211 - protein, 302 aa, Clostridium perfringens
GRDISNEVVTSLVATPNSINDGGNVQVRLEFKENHQRNIQSGDTITVKWTNSGEVFFEGYEKTIPLYIKDQNVGQAVIEKTGATLTFNDKIDKLDDVGGWATFTLQGRNITSGNHEHTGIAYIISGSKRADVNITKPESGTTSVFYYKTGSMYTNDTNHVNWWLLVNPSKVYSEKNVYIQDEIQGGQTLEPDSFEIVVTWYDGYVEKFKGKEAIREFHNKYPNSNISVSENKITVNISQEDSTQKFINIFYKTKITNPKQKEFVNNTKAWFKEYNKPAVNGESFNHSVQNINADAGVNGTVK

SEQ ID NO: 212 - protein, 114 aa, Artificial Sequence
  Description of Artificial Sequence Synthetic polypeptide
QVQLQESGGGLVQAGGSLRLSCAASGSIFSIRVMGWYRQAPGKQRELVATMSRGGTINYADSVRGRFTISRDNAKSTVYLQMNSLKPEDTAVYYCAALLDSYYWGQGTQVTVSS

SEQ ID NO: 213 - protein, 114 aa, Artificial Sequence
  Description of Artificial Sequence Synthetic polypeptide
QVQLQESGGGLVQAGGSLRLSCVVSGSIMSIRVMGWYRQAPGKQRELVATMSRGNTINYADSVRGRFTISRDNAKSTVFLEMNSLKPEDTAVYYCAALLDSYYWGQGTQVTVSS

SEQ ID NO: 214 - protein, 114 aa, Artificial Sequence
  Description of Artificial Sequence Synthetic polypeptide
QVQLQESGGGLVQAGGSLRLSCAASASIISIRVMGWYRQAPGKQRELVATMSRGGTINYADSVRGRFTISRDNAKSTVYLQMNSLKPEDTDVYYCAALLDSYYWGQGTQVTVSS

SEQ ID NO: 215 - protein, 114 aa, Artificial Sequence
  Description of Artificial Sequence Synthetic polypeptide
QVQLQESGGGLVQAGGSLRLSCVVSGSIMSIRVMGWYRQAPGKQRELVATMSRGGTINYADSVRGRFTISRDNAKSTVYLQMNSLKPEDTAVYYCTALLDSYYWGQGTQVTVSS

SEQ ID NO: 216 - protein, 114 aa, Artificial Sequence
  Description of Artificial Sequence Synthetic polypeptide
QVQLQESGGGLVQAGGSLRLSCAASGSIFSIRVMGWYRQAPGKQRELVATMSRGGTINYADSVRGRFTISRDNAKSTVYLQMNSLKPEDTAVYYCTALLDSYYWGQGTQVTVSS

SEQ ID NO: 217 - protein, 114 aa, Artificial Sequence
  Description of Artificial Sequence Synthetic polypeptide
QVQLQESGGGLVQAGGSLRLSCVVSGSIMSIRVMGWYRQAPGKQRELVATMSRGGTINYADSVRGRFTISRDNAKNTVYLQMTSLKPEDTAVYYCTALLDSYYWGQGTQVTVSS

SEQ ID NO: 218 - protein, 114 aa, Artificial Sequence
  Description of Artificial Sequence Synthetic polypeptide
QVQLQESGGGLVQAGGSLRLSCAASGSIFSIRVMGWYRQAPGKQRELVATMSRGGTINYADSVRGRFTISRDNAKSTVYLQMNSLKPEDTDVYYCAALLDSYYWGQGTQVTVSS

SEQ ID NO: 219 - protein, 114 aa, Artificial Sequence
  Description of Artificial Sequence Synthetic polypeptide
QVQLQESGGGLVQAGGSLRLSCVVSGSIMSIRVMGWYRQAPGKQRELVATMSRGNTINYADSVRGRFTISRDNAKNTVYLQMTSLKPEDTAVYYCAALLDSYYWGQGTQVTVSS

SEQ ID NO: 220 - protein, 114 aa, Artificial Sequence
  Description of Artificial Sequence Synthetic polypeptide
QVQLQESGGGLVQAGGSLRLSCAASASIFSIRVMGWYRQAPGKQRELVATMSRGNTINYADSVRGRFTISRDNAKSTVYLQMTSLKPEDTAVYYCAALLDSYYWGQGTQVTVSS

SEQ ID NO: 221 - protein, 114 aa, Artificial Sequence
  Description of Artificial Sequence Synthetic polypeptide
QVQLQESGGGLVQAGGSLRLSCVVSGSIMSIRVMGWYRQAPGKQRELVATMSRGGTINYADSVRGRFTISRDNAKSTVYLQMNSLKPEDTAVYYCAALLDSYYWGQGTQVTVSS

SEQ ID NO: 222 - protein, 114 aa, Artificial Sequence
  Description of Artificial Sequence Synthetic polypeptide
QVQLQESGGGLVQAGGSLRLSCAASASIISIRVMGWYRQAPGKQRELVATMSRGGTINYADSVRGRFTISRDNAKSTVYLQMNSLKPEDTAVYYCTALLDSYYWGQGTQVTVSS

SEQ ID NO: 223 - protein, 114 aa, Artificial Sequence
  Description of Artificial Sequence Synthetic polypeptide
QVQLQESGGGLVQAGGSLRLSCAASASIISIRVMGWYRQAPGKQRELVATMSRGGTINYADSVRGRFTISRDNAKSTVYLQMNSLKPEDTAVYYCAALLDSYYWGQGTQVTVSS

SEQ ID NO: 224 - protein, 114 aa, Artificial Sequence
  Description of Artificial Sequence Synthetic polypeptide
QVQLQESGGGLVQAGGSLRLSCAASASIMSIRVMGWYRQAPGKQRELVATMSRGNTINYADSVRGRFTISRDNAKSTVFLEMNSLKPEDTAVYYCAALLDSYYWGQGTQVTVSS

SEQ ID NO: 225 - protein, 114 aa, Artificial Sequence
  Description of Artificial Sequence Synthetic polypeptide
QVQLQESGGGLVQAGGSLRLSCAASASIMSIRVMGWYRQAPGKQRELVATMSRGGTINYADSVRGRFTISRDNAKSTVYLQMNSLKPEDTDVYYCAALLDSYYWGQGTQVTVSS

SEQ ID NO: 226 - protein, 114 aa, Artificial Sequence
  Description of Artificial Sequence Synthetic polypeptide
QVQLQESGGGLVQAGGSLRLSCVVSGSIMSIRVMGWYRQAPGKQRELVATMSRGGTINYADSVRGRFTISRDNAKSTVYLQMNSLKPEDTDVYYCAALLDSYYWGQGTQVTVSS

SEQ ID NO: 227 - protein, 114 aa, Artificial Sequence
  Description of Artificial Sequence Synthetic polypeptide
QVQLQESGGGLVQAGGSLRLSCAASASIISIRVMGWYRQAPGKQRELVATMSRGGTINYADSVRGRFTISRDNAKSTVYLQMTSLKPEDTAVYYCAALLDSYYWGQGTQVTVSS

SEQ ID NO: 228 - protein, 114 aa, Artificial Sequence
  Description of Artificial Sequence Synthetic polypeptide
QVQLQESGGGLVQAGGSLRLSCVVSGSIMSIRVMGWYRQAPGKQRELVATMSRGNTINYADSVKGRFTISRDNAKSTVFLQMNSLKPEDTDVYYCAALLDSYYWGQGTQVTVSS

SEQ ID NO: 229 - protein, 114 aa, Artificial Sequence
  Description of Artificial Sequence Synthetic polypeptide
QVQLQESGGGLVQAGGSLRLSCAASASIMSIRVMGWYRQAPGKQRELVATMSRGNTINYADSVRGRFTISRDNAKSTVYLQMNSLKPEDTAVYYCAALLDSYYWGQGTQVTVSS

SEQ ID NO: 230 - protein, 114 aa, Artificial Sequence
  Description of Artificial Sequence Synthetic polypeptide
QVQLQESGGGLVQAGGSLRLSCVVSGSIMSIRVMGWYRQAPGKQRELVATMSRGNTINYADSVRGRFTISRDNAKSTVYLQMNSLKPEDTAVYYCAALLDSYYWGQGTQVTVSS

SEQ ID NO: 231 - protein, 114 aa, Artificial Sequence
  Description of Artificial Sequence Synthetic polypeptide
QVQLQESGGGLVQAGGSLRLSCVVSGSIISIRVMGWYRQAPGKQRELVATMSRGGTINYADSVRGRFTISRDNAKSTVFLEMNSLKPEDTAVYYCAALLDSYYWGQGTQVTVSS

SEQ ID NO: 232 - protein, 114 aa, Artificial Sequence
  Description of Artificial Sequence Synthetic polypeptide
QVQLQESGGGLVQAGGSLRLSCAASASIISIRVMGWYRQAPGKQRELVATMSRGNTINYADSVRGRFTISRDDAKNTVYLQMNSLRPDDTAVYYCAALLDSYYWGQGTQVTVSS

SEQ ID NO: 233 - protein, 123 aa, Artificial Sequence
  Description of Artificial Sequence Synthetic polypeptide
QVQLQESGGGLVQAGGSLRLSCAASGSIFSINVMGWYRQAPGKQRDLVALITSGGSTTYADSVKGRFTISRDNAKNTVYLQMNSLKPEDTAVYYCNAAQSRTSWLFPDEYDYWGQGTQVTVSS

SEQ ID NO: 234 - protein, 126 aa, Artificial Sequence
  Description of Artificial Sequence Synthetic polypeptide
QVQLQESGGGLVQAGGSLRLSCAASGRTFSIYAMGWFRQAPGKEREFVAVINRGGGTTTYADSVKGRFTISRDNTKNTVSLQMNSLKPDDTAVYYCAADRVTDTYYYLNPESYDYWGQGTQVTVSS

SEQ ID NO: 235 - protein, 116 aa, Artificial Sequence
  Description of Artificial Sequence Synthetic polypeptide
QVQLQESGGGLVQAGGSLRLSCAASGSGRRVGYMAWYRQTPGKQRESVATISRAGATKYADSVKDRFTISRDNAKDTVYLQMNSLKPDDTAVYYCFASLIDAGTYWGQGTQVTVSS

SEQ ID NO: 236 - protein, 126 aa, Artificial Sequence
  Description of Artificial Sequence Synthetic polypeptide
QVQLQESGGGLVQAGGSLRLSCAASGRTFSIYAMGWFRQAPGKEREFVAVINRSGGTTTYADSVKGRFTISRDNTKNTVSLQMNSLKPDDTAVYYCAADRVTDTYYYLNPESYDYWGQGTQVTVSS

SEQ ID NO: 237 - protein, 111 aa, Artificial Sequence
  Description of Artificial Sequence Synthetic polypeptide
QVQLQESGGGLVQAGGSLRLSCAASGMSFSLGTIYWYRQAPGKQREFVAFITNADTTMYANSVKGRFTISRDNGKNTVFLLMNNLKPEDSAVYYCNTATSWGQGTQVTVSS

SEQ ID NO: 238 - protein, 116 aa, Artificial Sequence
  Description of Artificial Sequence Synthetic polypeptide
QVQLQESGGGLVQAGGSLRVSCAASGSGRRVGYMAWYRQTPGKQRELVATISRAGATNYADSVKDRFTISRDNAKNTVYLQMNSLKPDDTAVYYCFASVFDAGTYWGQGTQVTVSS

SEQ ID NO: 239 - protein, 116 aa, Artificial Sequence
  Description of Artificial Sequence Synthetic polypeptide
QVQLQESGGGLVQAGGSLRLSCAASGSGRRVGYMAWYRQTPGKQRELVATISRAGATNYADSVKDRFTISRDNAKNTVYLQMNSLKPDDTAVYYCFASIFDAGTYWGQGTQVTVSS

SEQ ID NO: 240 - protein, 116 aa, Artificial Sequence
  Description of Artificial Sequence Synthetic polypeptide
QVQLQESGGGLVQAGGSLRLSCVASGSGSRINYMAWHRQTPGRQRELVAVINRTGAANYARSVKDRFTISRDNAKNTVYLQMNDLKPDDTAIYYCFASYLGAGAYWGQGTQVTVSS

SEQ ID NO: 241 - protein, 118 aa, Artificial Sequence
  Description of Artificial Sequence Synthetic polypeptide
QVQLQESGGGLVQAGGSLRLSCAASGRTFSTYTVGWFRQAPGKEREFVASITWNGGTILYADSVKGRFTISRDNAKNTVLLQMNSLKPEDTAVYYCVMGAAGQGWRYWGQGTQVTVSS

SEQ ID NO: 242 - protein, 116 aa, Artificial Sequence
  Description of Artificial Sequence Synthetic polypeptide
QVQLQESGGGLVQAGGSLRLSCVASGSGSRINYMAWHRQTPGRQRELVAVINRTGAAKYADSVKDRFTVSRDNAENTVYLQMNDLKPDDTAVYYCWASYLGAGTYWGQGIQVTVSS

SEQ ID NO: 243 - protein, 112 aa, Artificial Sequence
  Description of Artificial Sequence Synthetic polypeptide
QVQLQESGGGLVQPGGSLRLSCAASGFTFSRNYMSWVRQAPGKGLEWVGSIYSDDSTNYAPSVKGRFTISRDNAANTLYLQMNSLKSEDTAVYYCSKEGGLRGQGTQVTVSS

SEQ ID NO: 244 - protein, 116 aa, Artificial Sequence
  Description of Artificial Sequence Synthetic polypeptide
QVQLQQSGGGLVQAGGSLRLSCAASGSGRRVGYMAWYRQTPGKQRELVATISRAGATNYADSVKDRFTISRDNAKNTVYLQMNSLKPDDTAVYYCFASVFDAGTYWGQGTQVTVSS

SEQ ID NO: 245 - protein, 116 aa, Artificial Sequence
  Description of Artificial Sequence Synthetic polypeptide
QVQLQESGGGLVQAGGSLRLSCAASGSGRRVGYMAWYRQTPGKQRELVATISRAGATNYADSVKDRFTISRDNAKNTVYLQMNSLKPDDTAVYYCFASLFDAGTYWGQGTQVTVSS

SEQ ID NO: 246 - protein, 123 aa, Artificial Sequence
  Description of Artificial Sequence Synthetic polypeptide
QVQLQESGGGLVQAGGSLRLSCAASGRTFSGRTMAWFRQAPGKEREFVAAITWSGGTTYYPDSVKGRFTISRDIPKNTLYLQMNSLKSEDTAVYYCASDGPWRATTPDAYDYWGQGTQVTVSS

SEQ ID NO: 247 - protein, 111 aa, Artificial Sequence
  Description of Artificial Sequence Synthetic polypeptide
QVQLQESGGGLVQAGGSLRLSCAASGSIGTIDSMGWYREAPGKRRELVAFIMFSGRTIYQDSVKGRFSISGDNAKKTVSLQMTSLKPEDTGVYYCYSNQYWGQGTQVTVSS

SEQ ID NO: 248 - protein, 118 aa, Artificial Sequence
  Description of Artificial Sequence Synthetic polypeptide
QVQLQQSGGGLVQPGGSLRLSCAASEFSLLFGTIGWFRQAPGKEREGVSCVSSSDGSTYYADSVKGRFTISRDKAKNTWYLQMHSLKPEDTAVYYCATRCTVVPGITWGQGTQVTVSS

SEQ ID NO: 249 - protein, 112 aa, Artificial Sequence
  Description of Artificial Sequence Synthetic polypeptide
QVQLQESGGGVVQAGGSLRLSCVAPGSITRVGGMGWYRQPPGKERELVALINEVGNTNYGDSVKGRFTISRDNAKKTVYLEMNSLKPEDTAVYYCWIPPIPWGQGTQVTVSS

SEQ ID NO: 250 - protein, 118 aa, Artificial Sequence
  Description of Artificial Sequence Synthetic polypeptide
QVQLQESGGGLVQPGGSLRLSCATSPFSLRLGVVGWFRQAPGREREGVSCISSSEGSTHYADSVKGRFTISRDNAKNTVYLQMNSLKPEDTAVYYCATRCTVVPGITWGQGTQVTVSS

SEQ ID NO: 251 - protein, 127 aa, Artificial Sequence
  Description of Artificial Sequence Synthetic polypeptide
QVQLQESGGGLVQAGDSLRLSCAASARTSSSRAMGWFRQTPVREREFVAAISWSGGRTAYADSVKGRFTLSKYDKDTVSLTMNSLKPEDTAVYYCAARRSDFTGDYAYSGRSAYDYWGQGTQVTVSS

SEQ ID NO: 252 - protein, 115 aa, Artificial Sequence
  Description of Artificial Sequence Synthetic polypeptide
QVQLQESGGGSVQAGGSLRLSCAASGSTFIFDKMDWYRQTPEKSRELVATLMSRGDPYYLDSVKGRFTITRDNAKNTVYLQMNSLKPEDTAVYVCRGRAGERVYWGQGTQVTVSS

SEQ ID NO: 253 - protein, 125 aa, Artificial Sequence
  Description of Artificial Sequence Synthetic polypeptide
QVQLQESGGGLVQPGGSLRLSCAASGRTFSGVIVGWFRQAPGKEREFLATTLWSGGSTYYTDSVKGRFTISRDVAKNMVYLQMNSLKPEDAAIYYCAAKYGGSLSYMHPTGYTYWGQGTQVTVSS

SEQ ID NO: 254 - protein, 123 aa, Artificial Sequence
  Description of Artificial Sequence Synthetic polypeptide
QVQLQESGGGLVQAGGSLRLSCAASRIVFTISTMAWFRQAPGKEREFVASINRSGALTSHANSVKGRFTISRDAAKNTVYLQMNSLKDEDTAIYYCAASKANMPALPANYDYWGQGTQVTVSS

SEQ ID NO: 255 - protein, 112 aa, Artificial Sequence
  Description of Artificial Sequence Synthetic polypeptide
QVQLQESGGGVVQAGGSLRLSCVAPGSITRLGSMGWYRQPPGKQRELVALITAVGNTNYGDSVKGRFTISRDNAKKMVYLEMNSLKPEDTAVYYCWIPPIPWGQGTQVTVSS

SEQ ID NO: 256 - protein, 112 aa, Artificial Sequence
  Description of Artificial Sequence Synthetic polypeptide
QVQLQESGGGVVQAGGSLRLSCVAPGSITRLGGMGWYRQTPGKQRELVALIDTVGNTNYGESVKGRFTISRDNAKKMVYLEMNSLKPEDTAVYYCWIPPIPWGQGTQVTVSS

SEQ ID NO: 257 - protein, 123 aa, Artificial Sequence
  Description of Artificial Sequence Synthetic polypeptide
QVQLQESGGGLVQAGDSLTLSCVASERAFMYNMAWFRQAPGKERDFVAVRNWNVERTNYADFAKGRFTISRDAAKKVMYLKMNNLKPEDTAVYYCATTRVWPTQHQMGQIEYWGQGTQVTVSS

SEQ ID NO: 258 - protein, 115 aa, Artificial Sequence
  Description of Artificial Sequence Synthetic polypeptide
QVQLQESGGGLVQAGGSLRLSCAASSSFNTMGWYRQAPGKQRELVAGITSGGTIKYGDSVKGRFTISGDNAKNTVYLQMDSLKPEDTAVYYCVADWQYGSTWNYWGQGTQVTVSS

SEQ ID NO: 259 - protein, 124 aa, Artificial Sequence
  Description of Artificial Sequence Synthetic polypeptide
QVQLQESGGGLVQAGDSLRLSCAASGRNFDYYSMGWFRQAPGNERIFVAAINWRGAVIDYPDSVKGRFTISRDNAKNRVYLQMNSLKPEDTAVYYCAAASSSSRLLEPIGYNYWGQGTQVTVSS

SEQ ID NO: 260 - protein, 120 aa, Artificial Sequence
  Description of Artificial Sequence Synthetic polypeptide
QVQLQESGGGLVQAGGSLRLSCAASGSMFSINDMTWYRQAPGKQREMVATISSGGTTDYTESVKGRFFVIRDNAKITVYLQMNKLRPEDSGVYYCAGNLKRSETSYYWKTGQGIQVTVSS

SEQ ID NO: 261 - protein, 122 aa, Artificial Sequence
  Description of Artificial Sequence Synthetic polypeptide
QVQLQESGGGLVQTGGSLKLSCATSGRTFSRYHMGWFRQAPGKEREFVAAISLSGGGTAFANFVEGRFTISRDNAKNTLYLQMNSLKPEDTAVYYCTADRHEWGRLMKGDYWGQGTQVTVSS

SEQ ID NO: 262 - protein, 127 aa, Artificial Sequence
  Description of Artificial Sequence Synthetic polypeptide
QVQLQESGGGSVQAGGSLTVSCSASGRTSNSYNMAWFRQGPGKERELVAAISWTGGFTSYTNSVKDRFTISRENAKNTVYLQMNSLKPEDTAVYYCAATSRSLTSAMTREIRAYDYWGQGTQVTVSS

SEQ ID NO: 263 - protein, 115 aa, Artificial Sequence
  Description of Artificial Sequence Synthetic polypeptide
QVQLQESGGGLVQAGGSLRLSCAASGSTFSFNKMDWYRQAPEKQRELVATFMNDGNTYYVDSVKGRFTISRDNAKNTVYLQMNSLKFEDTAVYYCRGRAGMEVYWGQGTQVTVSS

SEQ ID NO: 264 - protein, 117 aa, Artificial Sequence
  Description of Artificial Sequence Synthetic polypeptide
QVQLQESGGGLVQPGGSLTLSCATSPLTLRLGPIGWFRQAPGKEREGVSCISSRDDKNYAESVKGRFTISRDNAKNMVYLQMNSLKPEDTAVYYCATRCTVVPGISWGQGTQVTVSS

SEQ ID NO: 265 - protein, 123 aa, Artificial Sequence
  Description of Artificial Sequence Synthetic polypeptide
QVQLQESGGGLVQAGDSLRLSCAASGRNFGYYTMGWFRQAPGNERIFVAAITWRGVIHHADSVKGRFTISRDNAKNTVYLQMNSLKPEDTAVYYCAAASSSSRPLEPIGYNYWGQGTQVTVSS

SEQ ID NO: 266 - protein, 126 aa, Artificial Sequence
  Description of Artificial Sequence Synthetic polypeptide
QVQLQESGGGLVQAGGSLRLSCTASGDIFSAAGMAWFRQTPGKERDLVAYVTWDGGTTRYKDSVKGRFTISRDNAKNTVLLQMNSLKPEDTAVYYCAAGNTGPFNLLHSSAQYAYWGQGTQVTVSS

SEQ ID NO: 267 - protein, 117 aa, Artificial Sequence
  Description of Artificial Sequence Synthetic polypeptide
QVQLQESGGGLVQAGGSLRLSCATSPLTLRLGAIGWFRQAPGKEREMVSCITSTEDKNYADSVKGRFTISRDNAKTMVYLQMNSLKLEDTAVYYCATRCTVVPGISWGQGTQVTVSS

SEQ ID NO: 268 - protein, 112 aa, Artificial Sequence
  Description of Artificial Sequence Synthetic polypeptide
QVQLQESGGGVVQAGGSLRLSCVAPGSITRIGGMGWYRQPPGKQRELVALINTVGNTNYGDSVKGRFTISRDNAKKTVYLEMNSLKPEDTAVYYCWIPPLPWGQGTQVTVSS

SEQ ID NO: 269 - protein, 119 aa, Artificial Sequence
  Description of Artificial Sequence Synthetic polypeptide
QVQLQQSGGGLVQAGGSLRLSCTASGRSFSRYIMGWFRQAPGKERESVARIAPSGGSAYYADSVKGRFTISRDNAKNTVYLQMNNLKSEDTAVYHCAARYDMDYEYKTWGPGTQVTVSS

SEQ ID NO: 270 - protein, 116 aa, Artificial Sequence
  Description of Artificial Sequence Synthetic polypeptide
QVQLQESGGGLVQAGGSLRLSCVASGSGSRIGFMAWHRQTPGRQRELVAVINRTGATRYADSVKDRFTISRDNAKNTVYLQMNDLKPDDTALYYCFASVVDAGTYWGQGTQVTVSS

SEQ ID NO: 271 - protein, 127 aa, Artificial Sequence
  Description of Artificial Sequence Synthetic polypeptide
QVQLQESGGGLVQPGGSLRVSCAASGLTFSDYAMGWFRQAPGQEREFVARISLTAASTLYADSVRGRFTISRDNAKNTVYLQMNSLRPDDTAVYYCAAQGRILRGRGLFKASDYDYWGQGTQVTVSS

SEQ ID NO: 272 - protein, 116 aa, Artificial Sequence
  Description of Artificial Sequence Synthetic polypeptide
QVQLQQSGGGSVQTGGSLALSCAASGTISIFDPMGWYRQAPGKQRELVASISEGSTNYANSVKGRFTISRDNAKKTVSLQMNSLEPADTAVYYCRLSRYYNSNIYWGQGTQVTVSS

SEQ ID NO: 273 - protein, 119 aa, Artificial Sequence
  Description of Artificial Sequence Synthetic polypeptide
QVQLQESGGGLVQAGGSLRLSCAASRNIYGINVIAWYRQAPGKQREMVARSANGGTTRYADSVKGRFTISRDNVKNIVYLQMSSLKPEDTAAYYCKAELYTLQHNYEYWGQGTQVTVSS

SEQ ID NO: 274 - protein, 116 aa, Artificial Sequence
  Description of Artificial Sequence Synthetic polypeptide
QVQLQESGGGSVQTGGSLALSCVASGTLSLFDPMGWYRQAPGKQRELVASISGLSTNYANSVKGRFTISRDDAKKTVSLQMNSLEPADTAVYYCHLSRYYNSNIYWGQGTQVTVSS

SEQ ID NO: 275 - protein, 120 aa, Artificial Sequence
  Description of Artificial Sequence Synthetic polypeptide
QVQLQESGGGLVQAGGSLRLSCAASGRVLSINAMGWYRQAPGKRREMVARITNGGSTNYAGSVKGRFTISRENTKNTMYLQMNSLKPEDTAVYYCLAEERPYYGGPLEYWGQGTQVTVSQ

SEQ ID NO: 276 - protein, 116 aa, Artificial Sequence
  Description of Artificial Sequence Synthetic polypeptide
QVQLQESGGGLVQAGGSLRLSCAASRTTFRVGTMAWFRQDPGKQRELVAGITSGGSTNYADSVKGRFTISRDNAKNTIYLQMNSLKPEDTGIYVCFANIVDRPVSWGQGTQVTVSS

SEQ ID NO: 277 - protein, 116 aa, Artificial Sequence
  Description of Artificial Sequence Synthetic polypeptide
QVQLQQSGGGAVQAGGSLTLSCVASGSGSRIGLMAWYRQTPGRQRELVAVIKGTGTTRYADSVKDRFTISRDNAKNTMYLQMNDLKPDDTALYYCFASVLGAGTYWGQGTQVTVSS

SEQ ID NO: 278 - protein, 116 aa, Artificial Sequence
  Description of Artificial Sequence Synthetic polypeptide
QVQLQESGGGSVQTGGSLALSCAASGTISLFDSMGWYRQAPGKQRELVASITEGSTNYANSVKGRFTISRDNAKKTVSLQMNSLEPADTAVYYCRLSRYYNSNIYWGQGTQVTVSS

SEQ ID NO: 279 - protein, 114 aa, Artificial Sequence
  Description of Artificial Sequence Synthetic polypeptide
QVQLQQSGGGLVQSGGSLRLSCAASETSLNFDDMRWYRQTPGKRREWVAIINTFPAGTTASYADSVKGRFTISKVNGENTVHLQMNRLKPEDTAVYYCNAGDYWGQGTQVTVSS

SEQ ID NO: 280 - protein, 116 aa, Artificial Sequence
  Description of Artificial Sequence Synthetic polypeptide
QVQLQESGGGLVQAGGSLRLSCTASGSDSSINYMGWYRQAPGKQRVLLAAISRDGRSNYADSVRGRFTISRDNAKNTVDLQMNSLKPEDTAVYYCYVDPLGRVPRWGQGTQVTVSS

SEQ ID NO: 281 - protein, 113 aa, Artificial Sequence
  Description of Artificial Sequence Synthetic polypeptide
QVQLQESGGGAVQAGGSLTLSCVASGTVNLMAWYRQTPGRQRELVAVIKGTGTTRYADSVKDRFTISRDNAKNTMYLQMNDLKPDDTALYYCFASVLGAGTYWGQGTQVTVSS

SEQ ID NO: 282 - protein, 113 aa, Artificial Sequence
  Description of Artificial Sequence Synthetic polypeptide
QVQLQESGGGLVQAGGSLRLSCAASGSIFSRNIILWHRQAPGKQRELVGGINTGGRTNYESSVKGRFTISRDNAKNTVYLQMDRLKPEDTAVYYCNAPSLGYWGQGTQVTVSS

SEQ ID NO: 283 - protein, 115 aa, Artificial Sequence
  Description of Artificial Sequence Synthetic polypeptide
QVQLQQSGGGLVQAGGSLRLSCVASGSGSINYMAWHRQTPGRQRELVAVINRTGAARYADSVKDRFTISRDNAENTMYLQMNDLKPDDTAVYYCFASALGAGVYWGQGTQVTVSS

SEQ ID NO: 284 - protein, 116 aa, Artificial Sequence
  Description of Artificial Sequence Synthetic polypeptide
QVQLQESGGGLVQPGGSLRLSCAASGSGWRVGYMAWYRQTPGKQRELVATISRAGATRYEDSVKDRFTISRDNAKNTVYLQMNSLKPDDTAVYYCFASIIDAGTYWGQGTPVTVSS

SEQ ID NO: 285 - protein, 123 aa, Artificial Sequence
  Description of Artificial Sequence Synthetic polypeptide
QVQLQESGGGLVQAGGSLRLSCTASGENFSTYVMGWFRQAPGKEREFVAAHNWRGGGTYYADSVKGRFTISRDHAKNTVYLEMNSLKPEDTAVYYCAARSGGSYTYTGSYHYWGQGTQVTVSS

SEQ ID NO: 286 - protein, 123 aa, Artificial Sequence
  Description of Artificial Sequence Synthetic polypeptide
QVQLQESGGGLVQAGDSLRLSCAAAGRTFSSYAMGWFRQAPGKEREFVATISRSGGSTYYADSVKGRFTISRDNAKNTVYLQMNSLKPEDTAVYYCAANRYGSSSYQGQYASWGQGTQVTVSS

SEQ ID NO: 287 - protein, 123 aa, Artificial Sequence
  Description of Artificial Sequence Synthetic polypeptide
QVQLQESGGGLVQAGGSLRLSCAASGRTFSSYHMGWFRQAPGKEREFVATISRSGGFTSYADSVKGRFTISRDNAKNTVWLQMNSLKPEDTAVYYCAAQQWPDPRNPNGYDYWGQGTQVTVSS

SEQ ID NO: 288 - protein, 123 aa, Artificial Sequence
  Description of Artificial Sequence Synthetic polypeptide
QVQLQESGGGLVQAGGSLRLACAASGRTFINYGMAWFRQSPGKEREFVAAVSISGAGTAYVEPVKDRFTISRDNTKNTLYLQMNTLKPEDTALYYCAAAKAGHWGRDANYDYWGQGTQVTVSS

SEQ ID NO: 289 - protein, 123 aa, Artificial Sequence
  Description of Artificial Sequence Synthetic polypeptide
QVQLQQSGGGLVQAGGSLRLSCSASGRTLTAYGMAWFRQSPGKEREFVAAVSLSGASTAYVEPVKDRFTISRDNTQNTVYLQMNSLKPEDTALYYCAAAKAGQWGRDAKYDYWGQGTQVTVSS

SEQ ID NO: 290 - protein, 128 aa, Artificial Sequence
  Description of Artificial Sequence Synthetic polypeptide
QVQLQQSGGGLVQAGGSLRLSCAASGRTFSTYAMGWFRQAPGKEREFVAGISWSGGRISYTDSVKGRFTISRDNAKNTVYLQMNSLKPEDTAVYYCTADLKGLWALGLPGHYASWDSWGQGTQVTVSS

SEQ ID NO: 291 - protein, 115 aa, Artificial Sequence
  Description of Artificial Sequence Synthetic polypeptide
QVQLQESGGGLVQPGGSLRLSCAASGSIGSINIMDWYRQAPGKQRDLVATFTSGGSTVYADSVKGRFTISRDNAKDTVYLQMNSLKPEDTAVYYCRARRGWAIYWGQGTQVTVSS

SEQ ID NO: 292 - protein, 123 aa, Artificial Sequence
  Description of Artificial Sequence Synthetic polypeptide
QVQLQQSGGGLVQAGDSLRLSCAASGRTFSSYGMGWFRQATGKEREFVAGISRTGSGTAYADSVKSRFTISRDNAKNTVYLQMNSLKAEDTAVYYCAADSGGSWGRGTTYDYWGQGTQVTVSS

SEQ ID NO: 293 - protein, 124 aa, Artificial Sequence
  Description of Artificial Sequence Synthetic polypeptide
QVQLQQSGGGSVQAGGSLRLSCRASARASSIGAMAWFRQAPGKDRELVAAVTAGADTTYYRDFVKGRFTLSRDNAKNTVYLQMNSLKLDDTAVYYCAAYNTAGWGEPHQSYRYWGQGTQVTVSS

SEQ ID NO: 294 - protein, 122 aa, Artificial Sequence
  Description of Artificial Sequence Synthetic polypeptide
QVQLQESGGGLVQAGGSLKLSCVASGLTFGNYDMAWFRQAPGKEREFVTHISSSGAYTSYAYFVKGRFTISRDIAKNTVYLQMNSLKPEDTAIYYCAGRRSVVVRSFDYDYWGQGTQVTVSS

SEQ ID NO: 295 - protein, 115 aa, Artificial Sequence
  Description of Artificial Sequence Synthetic polypeptide
QVQLQQSGGGLVHPGGSLRLSCAASGRIFNANGMYWYRQAPGKQRELVASLYRSGSTNYLDSVKGRFTISRDNAKNTVYLQMNSLKPEDTAVYYCNVNWALHDSWGQGTQVTVSS

SEQ ID NO: 296 - protein, 123 aa, Artificial Sequence
  Description of Artificial Sequence Synthetic polypeptide
QVQLQESGGGLVQAGDSLRLSCAASERTFSSDGMAWFRQATGKEREFVAGISRTGSATAYAEFVKSRFTISRDNAKNTVYLQMNSLKAEDTAVYYCAANSGGHWWRGATYDYWGQGTQVTVSS

SEQ ID NO: 297 - protein, 115 aa, Artificial Sequence
  Description of Artificial Sequence Synthetic polypeptide
QVQLQESGGGLVQAGGSLRLSCTASGTIFSANGMYWYRQALGQRRELVASLYRDGSTNYADSVKGRFTISRDNAKNTVYLQMNSLKPEDTAVYYCNVNWALHDSWGQGTQVTVSS

SEQ ID NO: 298 - protein, 125 aa, Artificial Sequence
  Description of Artificial Sequence Synthetic polypeptide
QVQLQESGGGVVQAGDSLRLSCTASTRASIVGAMAWFRQAPGRNRDIVAAIAAGSPSTPYYADSVKGRFAISRDNAKNTVYLQMNSLKSEDTAIYYCAAYNTANWGQPHQSYRHWGQGIQVTVSS

SEQ ID NO: 299 - protein, 118 aa, Artificial Sequence
  Description of Artificial Sequence Synthetic polypeptide
QVQLQESGGGLVQPGGSLRLSAAASGSILNINVMAWFRQAPGKQREWVASIYRDGSTYYADSVKGRFTISRDNAKNTVYLQMNSLKPEDTAVYYCNVVTYGSNRRDFWGQGTQVTVST

SEQ ID NO: 300 - protein, 123 aa, Artificial Sequence
  Description of Artificial Sequence Synthetic polypeptide
QVQLQESGGGLVQAGDSLRLSCAASGRTFSSYAMGWFRQAPGKDREFVSTISRSGGSTYYADSVKGRFTISRDNAKNTVYLQMNSLKPEDTAVYYCAANRYGSSSYQGQYGSWGQGTQVTVSS

SEQ ID NO: 301 - protein, 123 aa, Artificial Sequence
  Description of Artificial Sequence Synthetic polypeptide
QVQLQQSGGGLVQAGDSLRLSCAASGRTFSSYAMGWFRQAPGKEREFVASISRSGGSTYYADSVKVRFTISRDNAKNTVYLQMNSLKPEDTAVYYCAANRYGSSSYQGQYDYWGQGTQVTVSS

SEQ ID NO: 302 - protein, 115 aa, Artificial Sequence
  Description of Artificial Sequence Synthetic polypeptide
QVQLQESGGGLVQAGGSLRLSCTASGTIFSINGMYWYRQALGKRRELVASLYRGGSTNYADSVKGRFTISRDNAKNTVYLQMNSLKPEDTAVYYCNVNWALQDSWGQGTQVTVSS

SEQ ID NO: 303 - protein, 115 aa, Artificial Sequence
  Description of Artificial Sequence Synthetic polypeptide
QVQLQESGGGLVQAGGSLRLSCAASTSDGSINVMDWYRQTPGKQRDLVATITSLGSQVYADSVKGRFTISRDNAKDTVYLQMNSLKPEDTAVYYCRARRGWAIYWGQGTQVTVSS

SEQ ID NO: 304 - protein, 128 aa, Artificial Sequence
  Description of Artificial Sequence Synthetic polypeptide
QVQLQQSGGGLVQAGGSLRLSCAASGRTFNIYAMGWFRQAPGKEREFVAGISDSGGSANYADSVKDRFTISMDNAKNTVYLQMNSLKPEDTAVYYCAADLTGLWALGLPGHYASWDSWGQGTQVTVSS

SEQ ID NO: 305 - protein, 117 aa, Artificial Sequence
  Description of Artificial Sequence Synthetic polypeptide
QVQLQESGGGLVQPGGSLRLSCAASGFTFRSSAMSWVRQVPGKGLEWVSSIGSDGENIYYADAVKGRFTISRDNAKNTMYLQMNSLKLEDTAVYYCQLGRTVLDYFKGQGTQVTVSS

SEQ ID NO: 306 - protein, 123 aa, Artificial Sequence
  Description of Artificial Sequence Synthetic polypeptide
QVQLQESGGGLVQPGGSLRLSCAASGRTFINYGMAWFRQSPGKEREFVAAVSSSGAGTAYVEPVKDRFTISRNNTKNTVYLQMNSLKPEDTALYYCAAAKAGQWGRYANYDYWGQGTQVTVSS

SEQ ID NO: 307 - protein, 123 aa, Artificial Sequence
  Description of Artificial Sequence Synthetic polypeptide
QVQLQQSGGGLVQAGDSLRLSCAASGRTFSSYAMGWFRQAPGKEREFVAAISRSGGTTYYADSVKGRFTISRDNAKNTVYLQMNTLKPEDTAVYYCAANPYGSSSYQGQYGSWGQGTQVTVSS

SEQ ID NO: 308 - protein, 123 aa, Artificial Sequence
  Description of Artificial Sequence Synthetic polypeptide
QVQLQQSGGGLVQAGDSLRLSCAASGRAFSGYAMGWFRQAPGREREFVAAISRGGGTTYYADSVKGRFTISRDNAKNTVYLQMNSLKPEDTAVYYCAANRYGSSSYQGQYGSWGQGTQVTVSS

SEQ ID NO: 309 - protein, 123 aa, Artificial Sequence
  Description of Artificial Sequence Synthetic polypeptide
QVQLQESGGGLVQAGGSLRLSCAASGRTFINYGMAWFRQSPGKEREFVAAVSSSGAGTAYVEPVKDRFTISRDNTKNTVYLQMDTLKPEDTALYYCAAAKAGHWGRDANYDYWGQGTQVTVSS

SEQ ID NO: 310 - protein, 116 aa, Artificial Sequence
  Description of Artificial Sequence Synthetic polypeptide
QVQLQESGGGMVEPGGSLRLSCAASGSISSITFMGWHRQAPGKEGEFVALIARSGTTTYADSVKGRFSISRDNAKNTVYLQMNNLKPEDTALYYCYVDRRGAVPTWGQGTQVTVSS

SEQ ID NO: 311 - protein, 116 aa, Artificial Sequence
  Description of Artificial Sequence Synthetic polypeptide
QVQLQQSGGGLVEPGGSLRLSCAASGSISSITFMGWHRQAPGEQGELVALIARSGTTTYADSVKGRFTISRDNAKNTVYLQMNNLKPEDTALYYCYVDRRDVVPTWGQGTQVTVSS

SEQ ID NO: 312 - protein, 117 aa, Artificial Sequence
  Description of Artificial Sequence Synthetic polypeptide
QVQLQESGGGLVQAGGSLRLSCAASGTGFPIITFMGYYRQAPGNQRELVAIISRGGVAKYGDSVKDRFTISRDNAKNTVYLEMNSLKPDDTAVYYCYADRFSGSPTWGQGTQVTVSS

SEQ ID NO: 313 - protein, 114 aa, Artificial Sequence
  Description of Artificial Sequence Synthetic polypeptide
QVQLQESGGGLVQPGGSLRLSCAASVSSIGTMGWFRQAPGKQPELVASISRVGTTNYANSVKGRFTVSRDNAQNTMYLQMNSLKPEDTAVYLCFANVISGPVYWGQGTQVTVSS

SEQ ID NO: 314 - protein, 123 aa, Artificial Sequence
  Description of Artificial Sequence Synthetic polypeptide
QVQLQESGGGLVQAGGSLRLSCAASTRFFSNYAMGWFRQAPGKEREFVAAISRDGAVPLSGNSVPGRFTISRDNAKNTLYLQMNSLKPEDSAVYYCAASRQGNPYAQTSYDYWGQGTQVTVSS

SEQ ID NO: 315 - protein, 120 aa, Artificial Sequence
  Description of Artificial Sequence Synthetic polypeptide
QVQLQESGGEVVAPGGSLSLSCVASGSADSIKIMGWYRQAPGKQRELVATITSGGTTEFAESVKGRFTISRDNAKNTLYMQMNSLSPEDTAVYYCNALVSRRDSAAYFAWGQGTQVTVSS

SEQ ID NO: 316 - protein, 115 aa, Artificial Sequence
  Description of Artificial Sequence Synthetic polypeptide
QVQLQESGGGLVQPGGSLRLSCAASESIVSITPMMWYRQAPGKQREWVAITTRDGAPAYADSVKGRFTISRDSAKNTVYLQMNYLKPEDTAVYFCKARKDSHDYWGQGTQVTVSS

SEQ ID NO: 317 - protein, 119 aa, Artificial Sequence
  Description of Artificial Sequence Synthetic polypeptide
QVQLQESGGGLVQAGGSLRLSCAASETIGSIQRMGWYRQAPGKQRELVATRTNGGTTNYGDSVRGRFTISVDVAKNTVYLQMNSLKPEDTAVYYCNAHIREYYSTYDYWGQGTQVTVSS

SEQ ID NO: 318 - protein, 120 aa, Artificial Sequence
  Description of Artificial Sequence Synthetic polypeptide
QVQLQESGGGLVQPGGSLRLSCSASGSISRIRDMAWHRQVPGKQRELVASISSGGSTNVADSLKGRFTISRDNGKNTMYLQMDSLKSEDTAVYYCNALFNPIDGPARYYWGQGTQVTVSS

SEQ ID NO: 319 - protein, 120 aa, Artificial Sequence
  Description of Artificial Sequence Synthetic polypeptide
QVQLQESGGGLVQPGGSLRLSCSASGSISRIYDMAWHRQVPGKQRELVAGISRGGSTNVADSLKGRFTISRDNGKNTVYLQMDNLKSEDTAVYYCTALFNPVDGTARYYWGQGTQVTVSS

SEQ ID NO: 320 - protein, 120 aa, Artificial Sequence
  Description of Artificial Sequence Synthetic polypeptide
QVQLQESGGGLVQAGGSLRLSCAASGTIFSINVMGWYRQAPGKQRELVASITSGGQIKYADSVKGRFTTSRDNAKNTVYLQMNSLKPEDTAVYYCNAASSTWPPRDYDYWGQGTQVTVSS

SEQ ID NO: 321 - protein, 120 aa, Artificial Sequence
  Description of Artificial Sequence Synthetic polypeptide
QVQLQESGGGLVQPGGSLRLSCAASRSISSIAAMGWYRQAPGKQRELVARITNGGSTNYADSVKGRFTISRDNAKNTVYLQMNSLKPEDTAVYYCNADERPYYGDSVLSWGQGTQVTVSS

SEQ ID NO: 322 - protein, 117 aa, Artificial Sequence
  Description of Artificial Sequence Synthetic polypeptide
QVQLQQSGGGLVQAGGSLRLSCAASGTGFPIITFMGYYRQTPGNQREEVALINRGGVAKYGDSVKDRFTISRDNAKNTVYLEMNNLKPDDTAVYYCYADRFSGSPTWGQGTQVTVSS

SEQ ID NO: 323 - protein, 125 aa, Artificial Sequence
  Description of Artificial Sequence Synthetic polypeptide
QVQLQESGGGLVQAGGSLRLSCAASGRTFSNYHMAWFRQAPGKEREFVAAISRGTSTTFYRDSVRDRFTISRDNAKNTAYLQMNSLKPEDTAVYYCAADADRSTTIYSRDIYDYWGQGTQVTVSS

SEQ ID NO: 324 - protein, 123 aa, Artificial Sequence
  Description of Artificial Sequence Synthetic polypeptide
QVQLQESGGGLVQAGDSLRLSCAASEGTFSNYRMGWFRQAPGKEREFVAAISRDGAVPLSGNSPLGRFTISRDNAKNTLYLQMNSLKPEDTAVYYCAASRQGLPYVETSYDYWGQGTQVTVSS

SEQ ID NO: 325 - protein, 116 aa, Artificial Sequence
  Description of Artificial Sequence Synthetic polypeptide
QVQLQESGGGLVQPGGSLRLSCVASGSISSITFMGWYRQVLGEQRELVALSARRGTTTYADSVKGRFTISRDNAKNTVYLQMNNLKPEDTALYYCYVDRRDEVPTWGQGTQVTVSS

SEQ ID NO: 326 - protein, 125 aa, Artificial Sequence
  Description of Artificial Sequence Synthetic polypeptide
QVQLQQSGGGLVQAGGSLRLSCAASGGTFSSYVMAWFRQAPGKEREFLAAIRWSRGSTYYADSVKGRFTVFRDTVENTVYLQMNSLKPEDTAVYYCAADGNPAKLVLDQYGMDYWGKGTLVTVSS

SEQ ID NO: 327 - protein, 116 aa, Artificial Sequence
  Description of Artificial Sequence Synthetic polypeptide
QVQLQQSGGGLVEPGGSLRLSCAASGSISEITYMGWHRQAPGEQRELVALIARVGTTRYADSVKGRFTISRDNAKNTVYLQMNNLKPEDTALYYCYVDQRGVVPTWGQGTQVTVSS

SEQ ID NO: 328 - protein, 124 aa, Artificial Sequence
  Description of Artificial Sequence Synthetic polypeptide
QVQLQESGGGSVQAGGSLRLSCRASARASSIGAMAWFRQAPGKDRELVAAVNAGADTTYYRDFVKGRFTISRDNAKNTVYLQMNSLKLDDTAVYYCAAYNTAGWGEPHQSYRYWGQGIQVTVSS

SEQ ID NO: 329 - protein, 121 aa, Artificial Sequence
  Description of Artificial Sequence Synthetic polypeptide
QVQLQESGGGLVQPGGSLSLSCAASGSIFIISTMGWYRQAPGKQRELVATITSGGSTNYADPVKGRFTISRDNAKNMVYLQMNSLKPEDTAVYYCNAEVHVWGVPGPRDYWGQGTQVTVSS

SEQ ID NO: 330 - protein, 123 aa, Artificial Sequence
  Description of Artificial Sequence Synthetic polypeptide
QVQLQESGGGLVQAGDSLRLSCAASGRTFSSYAMGWFRQAPGKEREFVATISRSGGSTYYADSVKGRFTISRDNAKNTVYLQMNSLKPEDTAVYYCAANPYGSSSYQGQYASWGQGTQVTVSS

SEQ ID NO: 331 - protein, 115 aa, Artificial Sequence
  Description of Artificial Sequence Synthetic polypeptide
QVQLQQSGGGLVQAGGSLRLSCAASGSIFSSNGMYWYRQAPGKQRELVASLYRSGSTNYADSVKGRFIISRDNAKNTVYLQMNSLKPEDTAVYYCNVNWALHDSWGQGTQVTVSS

SEQ ID NO: 332 - protein, 123 aa, Artificial Sequence
  Description of Artificial Sequence Synthetic polypeptide
QVQLQESGGGLVQAGGSLRLSCAASGRTFSAYGMAWFRQSPGKEREFVAAVSGGGGGTAYAEPVKDRFTISRDNAKNTVYLQMNTLKPEDTALYYCAAATAGHWGRDANYDYWGQGTQVTVSS

SEQ ID NO: 333 - protein, 115 aa, Artificial Sequence
  Description of Artificial Sequence Synthetic polypeptide
QVQLQESGGGLVQAGGSLRLSCAASGSIFSSNGMYWYRQAPGKQRELVASLFRSGSTNYADSVKGRFTISRDNAQNTVYLRMNSLKPEDTAVYYCNVNWALHDSWGPGTQVTVSS

SEQ ID NO: 334 - protein, 123 aa, Artificial Sequence
  Description of Artificial Sequence Synthetic polypeptide
QVQLQESGGGLVQAGDSLRLSCAASGRTFSSYAMGWFRQAPGKEREFVAAISRSGGTTYYADSVKGRFTISRDNAKNTVYLQMNTLKPEDTAVYYCAANPYGSSSYQGQYGSWGQGTQVTVSS

SEQ ID NO: 335 - protein, 115 aa, Artificial Sequence
  Description of Artificial Sequence Synthetic polypeptide
QVQLQESGGGLVQPGGSLRLSCAASGIIHSINVMGWYRQAPGKQRELVAIISSGGRTTYADSVKGRSTITGDNDKNTVYLQMNSLKPEDTAVYYCTMVWGLRYYWGQGTQVTVSS

SEQ ID NO: 336 - protein, 115 aa, Artificial Sequence
  Description of Artificial Sequence Synthetic polypeptide
QVQLQQSGGGFVRPGESLTLSCAASTSIFSSNGMYWYRQAPGKRRELVASLFRSGSTNYADSVKGRFTISRDNAKNTVYLQMNSLKPEDTAVYYCNVNWALHDSWGQGTQVTVSS

SEQ ID NO: 337 - protein, 123 aa, Artificial Sequence
  Description of Artificial Sequence Synthetic polypeptide
QVQLQESGGGLVQAGDSLRLSCAASGRTFSSYAMAWFRQAPGKEREFVAAISRGGGTTYYADSVKGRFTISRDNAKNTVYLQMNSLKPEDTAVYYCAANPYGSSSYQGQYGSWGQGTQVTVSS

SEQ ID NO: 338 - protein, 115 aa, Artificial Sequence
  Description of Artificial Sequence Synthetic polypeptide
QVQLQESGGGLVQAGGSLRLSCAASGSIFSSNGMYWYRQAPGKQRELVASLYRSGSTNYADSVKGRFIISRDNAKNTVYLQMNSLKPEDTAVYYCNVNWALHDSWGQGTQVTVSS

SEQ ID NO: 339 - protein, 115 aa, Artificial Sequence
  Description of Artificial Sequence Synthetic polypeptide
QVQLQQSGGGEVQPGGSLRLSCAASGSIFSSNGMYWYRQAPGKQRELVASLYRSGSTNYADSVKGRFIISRDNAKNTVYLQMNSLKPEDTAVYYCNVNWALHDSWGQGTQVTVSS

SEQ ID NO: 340 - protein, 115 aa, Artificial Sequence
  Description of Artificial Sequence Synthetic polypeptide
QVQLQESGGGLVQAGGSLRLSCAASRSILSANGMYWYRQAPGKQRELVASLYRSGSTDYADSVKGRFTISRDDSRDTMYLQMNSLKPEDTAVYYCNVNWALHDSWGQGTQVTVSS

SEQ ID NO: 341 - protein, 9 aa, Artificial Sequence
  Description of Artificial Sequence Synthetic peptide
ASIISIRVM

SEQ ID NO: 342 - protein, 9 aa, Artificial Sequence
  Description of Artificial Sequence Synthetic peptide
GSIMSIRVM

SEQ ID NO: 343 - protein, 9 aa, Artificial Sequence
  Description of Artificial Sequence Synthetic peptide
GSIFSIRVM

SEQ ID NO: 344 - protein, 9 aa, Artificial Sequence
  Description of Artificial Sequence Synthetic peptide
ASIFSIRVM

SEQ ID NO: 345 - protein, 9 aa, Artificial Sequence
  Description of Artificial Sequence Synthetic peptide
GSIMSIRVM

SEQ ID NO: 346 - protein, 9 aa, Artificial Sequence
  Description of Artificial Sequence Synthetic peptide
ASIMSIRVM

SEQ ID NO: 347 - protein, 9 aa, Artificial Sequence
  Description of Artificial Sequence Synthetic peptide
ASIMSIRVM

SEQ ID NO: 348 - protein, 9 aa, Artificial Sequence
  Description of Artificial Sequence Synthetic peptide
GSIMSIRVM

SEQ ID NO: 349 - protein, 9 aa, Artificial Sequence
  Description of Artificial Sequence Synthetic peptide
GSIISIRVM

SEQ ID NO: 350 - protein, 9 aa, Artificial Sequence
  Description of Artificial Sequence Synthetic peptide
ASIISIRVM

SEQ ID NO: 351 - protein, 9 aa, Artificial Sequence
  Description of Artificial Sequence Synthetic peptide
GSIFSINVM

SEQ ID NO: 352 - protein, 9 aa, Artificial Sequence
  Description of Artificial Sequence Synthetic peptide
GRTFSIYAM

SEQ ID NO: 353 - protein, 9 aa, Artificial Sequence
  Description of Artificial Sequence Synthetic peptide
GSGRRVGYM

SEQ ID NO: 354 - protein, 9 aa, Artificial Sequence
  Description of Artificial Sequence Synthetic peptide
GRTFSIYAM

SEQ ID NO: 355 - protein, 9 aa, Artificial Sequence
  Description of Artificial Sequence Synthetic peptide
GMSFSLGTI

SEQ ID NO: 356 - protein, 9 aa, Artificial Sequence
  Description of Artificial Sequence Synthetic peptide
GSGRRVGYM

SEQ ID NO: 357 - protein, 9 aa, Artificial Sequence
  Description of Artificial Sequence Synthetic peptide
GSGRRVGYM

SEQ ID NO: 358 - protein, 9 aa, Artificial Sequence
  Description of Artificial Sequence Synthetic peptide
GSGSRINYM

SEQ ID NO: 359 - protein, 9 aa, Artificial Sequence
  Description of Artificial Sequence Synthetic peptide
GRTFSTYTV

SEQ ID NO: 360 - protein, 9 aa, Artificial Sequence
  Description of Artificial Sequence Synthetic peptide
GSGSRINYM

SEQ ID NO: 361 - protein, 9 aa, Artificial Sequence
  Description of Artificial Sequence Synthetic peptide
GFTFSRNYM

SEQ ID NO: 362 - protein, 9 aa, Artificial Sequence
  Description of Artificial Sequence Synthetic peptide
GSGRRVGYM

SEQ ID NO: 363 - protein, 9 aa, Artificial Sequence
  Description of Artificial Sequence Synthetic peptide
GSGRRVGYM

SEQ ID NO: 364 - protein, 9 aa, Artificial Sequence
  Description of Artificial Sequence Synthetic peptide
GRTFSGRTM

SEQ ID NO: 365 - protein, 9 aa, Artificial Sequence
  Description of Artificial Sequence Synthetic peptide
GSIGTIDSM

SEQ ID NO: 366 - protein, 9 aa, Artificial Sequence
  Description of Artificial Sequence Synthetic peptide
EFSLLFGTI

SEQ ID NO: 367 - protein, 9 aa, Artificial Sequence
  Description of Artificial Sequence Synthetic peptide
GSITRVGGM

SEQ ID NO: 368 - protein, 9 aa, Artificial Sequence
  Description of Artificial Sequence Synthetic peptide
PFSLRLGVV

SEQ ID NO: 369 - protein, 9 aa, Artificial Sequence
  Description of Artificial Sequence Synthetic peptide
ARTSSSRAM

SEQ ID NO: 370 - protein, 9 aa, Artificial Sequence
  Description of Artificial Sequence Synthetic peptide
GSTFIFDKM

SEQ ID NO: 371 - protein, 9 aa, Artificial Sequence
  Description of Artificial Sequence Synthetic peptide
GRTFSGVIV

SEQ ID NO: 372 - protein, 9 aa, Artificial Sequence
  Description of Artificial Sequence Synthetic peptide
RIVFTISTM

SEQ ID NO: 373 - protein, 9 aa, Artificial Sequence
  Description of Artificial Sequence Synthetic peptide
GSITRLGSM

SEQ ID NO: 374 - protein, 9 aa, Artificial Sequence
  Description of Artificial Sequence Synthetic peptide
GSITRLGGM

SEQ ID NO: 375 - protein, 8 aa, Artificial Sequence
  Description of Artificial Sequence Synthetic peptide
ERAFMYNM

SEQ ID NO: 376 - protein, 6 aa, Artificial Sequence
  Description of Artificial Sequence Synthetic peptide
SSFNTM

SEQ ID NO: 377 - protein, 9 aa, Artificial Sequence
  Description of Artificial Sequence Synthetic peptide
GRNFDYYSM

SEQ ID NO: 378 - protein, 9 aa, Artificial Sequence
  Description of Artificial Sequence Synthetic peptide
GSMFSINDM

SEQ ID NO: 379 - protein, 9 aa, Artificial Sequence
  Description of Artificial Sequence Synthetic peptide
GRTFSRYHM

SEQ ID NO: 380 - protein, 9 aa, Artificial Sequence
  Description of Artificial Sequence Synthetic peptide
GRTSNSYNM

SEQ ID NO: 381 - protein, 9 aa, Artificial Sequence
  Description of Artificial Sequence Synthetic peptide
GSTFSFNKM

SEQ ID NO: 382 - protein, 9 aa, Artificial Sequence
  Description of Artificial Sequence Synthetic peptide
PLTLRLGPI

SEQ ID NO: 383 - protein, 9 aa, Artificial Sequence
  Description of Artificial Sequence Synthetic peptide
GRNFGYYTM

SEQ ID NO: 384 - protein, 9 aa, Artificial Sequence
  Description of Artificial Sequence Synthetic peptide
GDIFSAAGM

SEQ ID NO: 385 - protein, 9 aa, Artificial Sequence
  Description of Artificial Sequence Synthetic peptide
PLTLRLGAI

SEQ ID NO: 386 - protein, 9 aa, Artificial Sequence
  Description of Artificial Sequence Synthetic peptide
GSITRIGGM

SEQ ID NO: 387 - protein, 9 aa, Artificial Sequence
  Description of Artificial Sequence Synthetic peptide
GRSFSRYIM

SEQ ID NO: 388 - protein, 9 aa, Artificial Sequence
  Description of Artificial Sequence Synthetic peptide
GSGSRIGFM

SEQ ID NO: 389 - protein, 9 aa, Artificial Sequence
  Description of Artificial Sequence Synthetic peptide
GLTFSDYAM

SEQ ID NO: 390 - protein, 9 aa, Artificial Sequence
  Description of Artificial Sequence Synthetic peptide
GTISIFDPM

SEQ ID NO: 391 - protein, 9 aa, Artificial Sequence
  Description of Artificial Sequence Synthetic peptide
RNIYGINVI

SEQ ID NO: 392 - protein, 9 aa, Artificial Sequence
  Description of Artificial Sequence Synthetic peptide
GTLSLFDPM

SEQ ID NO: 393 - protein, 9 aa, Artificial Sequence
  Description of Artificial Sequence Synthetic peptide
GRVLSINAM

SEQ ID NO: 394 - protein, 9 aa, Artificial Sequence
  Description of Artificial Sequence Synthetic peptide
RTTFRVGTM

SEQ ID NO: 395 - protein, 9 aa, Artificial Sequence
  Description of Artificial Sequence Synthetic peptide
GSGSRIGLM

SEQ ID NO: 396 - protein, 9 aa, Artificial Sequence
  Description of Artificial Sequence Synthetic peptide
GTISLFDSM

SEQ ID NO: 397 - protein, 9 aa, Artificial Sequence
  Description of Artificial Sequence Synthetic peptide
ETSLNFDDM

SEQ ID NO: 398 - protein, 9 aa, Artificial Sequence
  Description of Artificial Sequence Synthetic peptide
GSDSSINYM

SEQ ID NO: 399 - protein, 6 aa, Artificial Sequence
  Description of Artificial Sequence Synthetic peptide
GTVNLM

SEQ ID NO: 400 - protein, 9 aa, Artificial Sequence
  Description of Artificial Sequence Synthetic peptide
GSIFSRNII

SEQ ID NO: 401 - protein, 8 aa, Artificial Sequence
  Description of Artificial Sequence Synthetic peptide
GSGSINYM

SEQ ID NO: 402 - protein, 9 aa, Artificial Sequence
  Description of Artificial Sequence Synthetic peptide
GSGWRVGYM

SEQ ID NO: 403 - protein, 9 aa, Artificial Sequence
  Description of Artificial Sequence Synthetic peptide
GENFSTYVM

SEQ ID NO: 404 - protein, 9 aa, Artificial Sequence
  Description of Artificial Sequence Synthetic peptide
GRTFSSYAM

SEQ ID NO: 405 - protein, 9 aa, Artificial Sequence
  Description of Artificial Sequence Synthetic peptide
GRTFSSYHM

SEQ ID NO: 406 - protein, 9 aa, Artificial Sequence
  Description of Artificial Sequence Synthetic peptide
GRTFINYGM

SEQ ID NO: 407 - protein, 9 aa, Artificial Sequence
  Description of Artificial Sequence Synthetic peptide
GRTLTAYGM

SEQ ID NO: 408 - protein, 9 aa, Artificial Sequence
  Description of Artificial Sequence Synthetic peptide
GRTFSTYAM

SEQ ID NO: 409 - protein, 9 aa, Artificial Sequence
  Description of Artificial Sequence Synthetic peptide
GSIGSINIM

SEQ ID NO: 410 - protein, 9 aa, Artificial Sequence
  Description of Artificial Sequence Synthetic peptide
GRTFSSYGM

SEQ ID NO: 411 - protein, 9 aa, Artificial Sequence
  Description of Artificial Sequence Synthetic peptide
ARASSIGAM

SEQ ID NO: 412 - protein, 9 aa, Artificial Sequence
  Description of Artificial Sequence Synthetic peptide
GLTFGNYDM

SEQ ID NO: 413 - protein, 9 aa, Artificial Sequence
  Description of Artificial Sequence Synthetic peptide
GRIFNANGM

SEQ ID NO: 414 - protein, 9 aa, Artificial Sequence
  Description of Artificial Sequence Synthetic peptide
ERTFSSDGM

SEQ ID NO: 415 - protein, 9 aa, Artificial Sequence
  Description of Artificial Sequence Synthetic peptide
GTIFSANGM

SEQ ID NO: 416 - protein, 9 aa, Artificial Sequence
  Description of Artificial Sequence Synthetic peptide
TRASIVGAM

SEQ ID NO: 417 - protein, 9 aa, Artificial Sequence
  Description of Artificial Sequence Synthetic peptide
GSILNINVM

SEQ ID NO: 418 - protein, 9 aa, Artificial Sequence
  Description of Artificial Sequence Synthetic peptide
GRTFSSYAM

SEQ ID NO: 419 - protein, 9 aa, Artificial Sequence
  Description of Artificial Sequence Synthetic peptide
GRTFSSYAM

SEQ ID NO: 420 - protein, 9 aa, Artificial Sequence
  Description of Artificial Sequence Synthetic peptide
GTIFSINGM

SEQ ID NO: 421 - protein, 9 aa, Artificial Sequence
  Description of Artificial Sequence Synthetic peptide
TSDGSINVM

SEQ ID NO: 422 - protein, 9 aa, Artificial Sequence
  Description of Artificial Sequence Synthetic peptide
GRTFNIYAM

SEQ ID NO: 423 - protein, 9 aa, Artificial Sequence
  Description of Artificial Sequence Synthetic peptide
GFTFRSSAM

SEQ ID NO: 424 - protein, 9 aa, Artificial Sequence
  Description of Artificial Sequence Synthetic peptide
GRTFINYGM

SEQ ID NO: 425 - protein, 9 aa, Artificial Sequence
  Description of Artificial Sequence Synthetic peptide
GRTFSSYAM

SEQ ID NO: 426 - protein, 9 aa, Artificial Sequence
  Description of Artificial Sequence Synthetic peptide
GRAFSGYAM

SEQ ID NO: 427 - protein, 9 aa, Artificial Sequence
  Description of Artificial Sequence Synthetic peptide
GRTFINYGM

SEQ ID NO: 428 - protein, 9 aa, Artificial Sequence
  Description of Artificial Sequence Synthetic peptide
GSISSITFM

SEQ ID NO: 429 - protein, 9 aa, Artificial Sequence
  Description of Artificial Sequence Synthetic peptide
GSISSITFM

SEQ ID NO: 430 - protein, 10 aa, Artificial Sequence
  Description of Artificial Sequence Synthetic peptide
GTGFPIITFM

SEQ ID NO: 431 - protein, 7 aa, Artificial Sequence
  Description of Artificial Sequence Synthetic peptide
VSSIGTM

SEQ ID NO: 432 - protein, 9 aa, Artificial Sequence
  Description of Artificial Sequence Synthetic peptide
TRFFSNYAM

SEQ ID NO: 433 - protein, 9 aa, Artificial Sequence
  Description of Artificial Sequence Synthetic peptide
GSADSIKIM

SEQ ID NO: 434 - protein, 9 aa, Artificial Sequence
  Description of Artificial Sequence Synthetic peptide
ESIVSITPM

SEQ ID NO: 435 - protein, 9 aa, Artificial Sequence
  Description of Artificial Sequence Synthetic peptide
ETIGSIQRM

SEQ ID NO: 436 - protein, 9 aa, Artificial Sequence
  Description of Artificial Sequence Synthetic peptide
GSISRIRDM

SEQ ID NO: 437 - protein, 9 aa, Artificial Sequence
  Description of Artificial Sequence Synthetic peptide
GSISRIYDM

SEQ ID NO: 438 - protein, 9 aa, Artificial Sequence
  Description of Artificial Sequence Synthetic peptide
GTIFSINVM

SEQ ID NO: 439 - protein, 9 aa, Artificial Sequence
  Description of Artificial Sequence Synthetic peptide
RSISSIAAM

SEQ ID NO: 440 - protein, 10 aa, Artificial Sequence
  Description of Artificial Sequence Synthetic peptide
GTGFPIITFM

SEQ ID NO: 441 - protein, 9 aa, Artificial Sequence
  Description of Artificial Sequence Synthetic peptide
GRTFSNYHM

SEQ ID NO: 442 - protein, 9 aa, Artificial Sequence
  Description of Artificial Sequence Synthetic peptide
EGTFSNYRM

SEQ ID NO: 443 - protein, 9 aa, Artificial Sequence
  Description of Artificial Sequence Synthetic peptide
GSISSITFM

SEQ ID NO: 444 - protein, 9 aa, Artificial Sequence
  Description of Artificial Sequence Synthetic peptide
GGTFSSYVM

SEQ ID NO: 445 - protein, 9 aa, Artificial Sequence
  Description of Artificial Sequence Synthetic peptide
GSISEITYM

SEQ ID NO: 446 - protein, 9 aa, Artificial Sequence
  Description of Artificial Sequence Synthetic peptide
ARASSIGAM

SEQ ID NO: 447 - protein, 9 aa, Artificial Sequence
  Description of Artificial Sequence Synthetic peptide
GSIFIISTM

SEQ ID NO: 448 - protein, 9 aa, Artificial Sequence
  Description of Artificial Sequence Synthetic peptide
GRTFSSYAM

SEQ ID NO: 449 - protein, 9 aa, Artificial Sequence
  Description of Artificial Sequence Synthetic peptide
GSIFSSNGM

SEQ ID NO: 450 - protein, 9 aa, Artificial Sequence
  Description of Artificial Sequence Synthetic peptide
GRTFSAYGM

SEQ ID NO: 451 - protein, 9 aa, Artificial Sequence
  Description of Artificial Sequence Synthetic peptide
GSIFSSNGM

SEQ ID NO: 452 - protein, 9 aa, Artificial Sequence
  Description of Artificial Sequence Synthetic peptide
GRTFSSYAM

SEQ ID NO: 453 - protein, 9 aa, Artificial Sequence
  Description of Artificial Sequence Synthetic peptide
GIIHSINVM

SEQ ID NO: 454 - protein, 9 aa, Artificial Sequence
  Description of Artificial Sequence Synthetic peptide
TSIFSSNGM

SEQ ID NO: 455 - protein, 9 aa, Artificial Sequence
  Description of Artificial Sequence Synthetic peptide
GRTFSSYAM

SEQ ID NO: 456 - protein, 9 aa, Artificial Sequence
  Description of Artificial Sequence Synthetic peptide
GSIFSSNGM

SEQ ID NO: 457 - protein, 9 aa, Artificial Sequence
  Description of Artificial Sequence Synthetic peptide
GSIFSSNGM

SEQ ID NO: 458 - protein, 9 aa, Artificial Sequence
  Description of Artificial Sequence Synthetic peptide
RSILSANGM

SEQ ID NO: 459 - protein, 7 aa, Artificial Sequence
  Description of Artificial Sequence Synthetic peptide
MSRGGTI

SEQ ID NO: 460 - protein, 7 aa, Artificial Sequence
  Description of Artificial Sequence Synthetic peptide
MSRGGTI

SEQ ID NO: 461 - protein, 7 aa, Artificial Sequence
  Description of Artificial Sequence Synthetic peptide
MSRGGTI

SEQ ID NO: 462 - protein, 7 aa, Artificial Sequence
  Description of Artificial Sequence Synthetic peptide
MSRGNTI

SEQ ID NO: 463 - protein, 7 aa, Artificial Sequence
  Description of Artificial Sequence Synthetic peptide
MSRGGTI

SEQ ID NO: 464 - protein, 7 aa, Artificial Sequence
  Description of Artificial Sequence Synthetic peptide
MSRGNTI

SEQ ID NO: 465 - protein, 7 aa, Artificial Sequence
  Description of Artificial Sequence Synthetic peptide
MSRGGTI

SEQ ID NO: 466 - protein, 7 aa, Artificial Sequence
  Description of Artificial Sequence Synthetic peptide
MSRGNTI

SEQ ID NO: 467 - protein, 7 aa, Artificial Sequence
  Description of Artificial Sequence Synthetic peptide
MSRGGTI

SEQ ID NO: 468 - protein, 7 aa, Artificial Sequence
  Description of Artificial Sequence Synthetic peptide
MSRGNTI

SEQ ID NO: 469 - protein, 7 aa, Artificial Sequence
  Description of Artificial Sequence Synthetic peptide
ITSGGST

SEQ ID NO: 470 - protein, 8 aa, Artificial Sequence
  Description of Artificial Sequence Synthetic peptide
INRGGGTT

SEQ ID NO: 471 - protein, 7 aa, Artificial Sequence
  Description of Artificial Sequence Synthetic peptide
ISRAGAT

SEQ ID NO: 472 - protein, 8 aa, Artificial Sequence
  Description of Artificial Sequence Synthetic peptide
INRSGGTT

SEQ ID NO: 473 - protein, 7 aa, Artificial Sequence
  Description of Artificial Sequence Synthetic peptide
ITNADTT

SEQ ID NO: 474 - protein, 7 aa, Artificial Sequence
  Description of Artificial Sequence Synthetic peptide
ISRAGAT

SEQ ID NO: 475 - protein, 7 aa, Artificial Sequence
  Description of Artificial Sequence Synthetic peptide
ISRAGAT

SEQ ID NO: 476 - protein, 7 aa, Artificial Sequence
  Description of Artificial Sequence Synthetic peptide
INRTGAA

SEQ ID NO: 477 - protein, 8 aa, Artificial Sequence
  Description of Artificial Sequence Synthetic peptide
ITWNGGTI

SEQ ID NO: 478 - protein, 7 aa, Artificial Sequence
  Description of Artificial Sequence Synthetic peptide
INRTGAA

SEQ ID NO: 479 - protein, 7 aa, Artificial Sequence
  Description of Artificial Sequence Synthetic peptide
IYSDDST

SEQ ID NO: 480 - protein, 7 aa, Artificial Sequence
  Description of Artificial Sequence Synthetic peptide
ISRAGAT

SEQ ID NO: 481 - protein, 7 aa, Artificial Sequence
  Description of Artificial Sequence Synthetic peptide
ISRAGAT

SEQ ID NO: 482 - protein, 8 aa, Artificial Sequence
  Description of Artificial Sequence Synthetic peptide
ITWSGGTT

SEQ ID NO: 483 - protein, 7 aa, Artificial Sequence
  Description of Artificial Sequence Synthetic peptide
IMFSGRT

SEQ ID NO: 484 - protein, 8 aa, Artificial Sequence
  Description of Artificial Sequence Synthetic peptide
VSSSDGST

SEQ ID NO: 485 - protein, 7 aa, Artificial Sequence
  Description of Artificial Sequence Synthetic peptide
INEVGNT

SEQ ID NO: 486 - protein, 8 aa, Artificial Sequence
  Description of Artificial Sequence Synthetic peptide
ISSSEGST

SEQ ID NO: 487 - protein, 8 aa, Artificial Sequence
  Description of Artificial Sequence Synthetic peptide
ISWSGGRT

SEQ ID NO: 488 - protein, 7 aa, Artificial Sequence
  Description of Artificial Sequence Synthetic peptide
LMSRGDP

SEQ ID NO: 489 - protein, 8 aa, Artificial Sequence
  Description of Artificial Sequence Synthetic peptide
TLWSGGST

SEQ ID NO: 490 - protein, 8 aa, Artificial Sequence
  Description of Artificial Sequence Synthetic peptide
INRSGALT

SEQ ID NO: 491 - protein, 7 aa, Artificial Sequence
  Description of Artificial Sequence Synthetic peptide
ITAVGNT

SEQ ID NO: 492 - protein, 7 aa, Artificial Sequence
  Description of Artificial Sequence Synthetic peptide
IDTVGNT

SEQ ID NO: 493 - protein, 8 aa, Artificial Sequence
  Description of Artificial Sequence Synthetic peptide
RNWNVERT

SEQ ID NO: 494 - protein, 7 aa, Artificial Sequence
  Description of Artificial Sequence Synthetic peptide
ITSGGTI

SEQ ID NO: 495 - protein, 8 aa, Artificial Sequence
  Description of Artificial Sequence Synthetic peptide
INWRGAVI

SEQ ID NO: 496 - protein, 7 aa, Artificial Sequence
  Description of Artificial Sequence Synthetic peptide
ISSGGTT

SEQ ID NO: 497 - protein, 8 aa, Artificial Sequence
  Description of Artificial Sequence Synthetic peptide
ISLSGGGT

SEQ ID NO: 498 - protein, 8 aa, Artificial Sequence
  Description of Artificial Sequence Synthetic peptide
ISWTGGFT

SEQ ID NO: 499 - protein, 7 aa, Artificial Sequence
  Description of Artificial Sequence Synthetic peptide
FMNDGNT

SEQ ID NO: 500 - protein, 7 aa, Artificial Sequence
  Description of Artificial Sequence Synthetic peptide
ISSRDDK

SEQ ID NO: 501 - protein, 7 aa, Artificial Sequence
  Description of Artificial Sequence Synthetic peptide
ITWRGVI

SEQ ID NO: 502 - protein, 8 aa, Artificial Sequence
  Description of Artificial Sequence Synthetic peptide
VTWDGGTT

SEQ ID NO: 503 - protein, 7 aa, Artificial Sequence
  Description of Artificial Sequence Synthetic peptide
ITSTEDK

SEQ ID NO: 504 - protein, 7 aa, Artificial Sequence
  Description of Artificial Sequence Synthetic peptide
INTVGNT

SEQ ID NO: 505 - protein, 8 aa, Artificial Sequence
  Description of Artificial Sequence Synthetic peptide
IAPSGGSA

SEQ ID NO: 506 - protein, 7 aa, Artificial Sequence
  Description of Artificial Sequence Synthetic peptide
INRTGAT

SEQ ID NO: 507 - protein, 8 aa, Artificial Sequence
  Description of Artificial Sequence Synthetic peptide
ISLTAAST

SEQ ID NO: 508 - protein, 6 aa, Artificial Sequence
  Description of Artificial Sequence Synthetic peptide
ISEGST

SEQ ID NO: 509 - protein, 7 aa, Artificial Sequence
  Description of Artificial Sequence Synthetic peptide
SANGGTT

SEQ ID NO: 510 - protein, 6 aa, Artificial Sequence
  Description of Artificial Sequence Synthetic peptide
ISGLST

SEQ ID NO: 511 - protein, 7 aa, Artificial Sequence
  Description of Artificial Sequence Synthetic peptide
ITNGGST

SEQ ID NO: 512 - protein, 7 aa, Artificial Sequence
  Description of Artificial Sequence Synthetic peptide
ITSGGST

SEQ ID NO: 513 - protein, 7 aa, Artificial Sequence
  Description of Artificial Sequence Synthetic peptide
IKGTGTT

SEQ ID NO: 514 - protein, 6 aa, Artificial Sequence
  Description of Artificial Sequence Synthetic peptide
ITEGST

SEQ ID NO: 515 - protein, 10 aa, Artificial Sequence
  Description of Artificial Sequence Synthetic peptide
INTFPAGTTA

SEQ ID NO: 516 - protein, 7 aa, Artificial Sequence
  Description of Artificial Sequence Synthetic peptide
ISRDGRS

SEQ ID NO: 517 - protein, 7 aa, Artificial Sequence
  Description of Artificial Sequence Synthetic peptide
IKGTGTT

SEQ ID NO: 518 - protein, 7 aa, Artificial Sequence
  Description of Artificial Sequence Synthetic peptide
INTGGRT

SEQ ID NO: 519 - protein, 7 aa, Artificial Sequence
  Description of Artificial Sequence Synthetic peptide
INRTGAA

SEQ ID NO: 520 - protein, 7 aa, Artificial Sequence
  Description of Artificial Sequence Synthetic peptide
ISRAGAT

SEQ ID NO: 521 - protein, 8 aa, Artificial Sequence
  Description of Artificial Sequence Synthetic peptide
HNWRGGGT

SEQ ID NO: 522 - protein, 8 aa, Artificial Sequence
  Description of Artificial Sequence Synthetic peptide
ISRSGGST

SEQ ID NO: 523 - protein, 8 aa, Artificial Sequence
  Description of Artificial Sequence Synthetic peptide
ISRSGGFT

SEQ ID NO: 524 - protein, 8 aa, Artificial Sequence
  Description of Artificial Sequence Synthetic peptide
VSISGAGT

SEQ ID NO: 525 - protein, 8 aa, Artificial Sequence
  Description of Artificial Sequence Synthetic peptide
VSLSGAST

SEQ ID NO: 526 - protein, 8 aa, Artificial Sequence
  Description of Artificial Sequence Synthetic peptide
ISWSGGRI

SEQ ID NO: 527 - protein, 7 aa, Artificial Sequence
  Description of Artificial Sequence Synthetic peptide
FTSGGST

SEQ ID NO: 528 - protein, 8 aa, Artificial Sequence
  Description of Artificial Sequence Synthetic peptide
ISRTGSGT

SEQ ID NO: 529 - protein, 8 aa, Artificial Sequence
  Description of Artificial Sequence Synthetic peptide
VTAGADTT

SEQ ID NO: 530 - protein, 8 aa, Artificial Sequence
  Description of Artificial Sequence Synthetic peptide
ISSSGAYT

SEQ ID NO: 531 - protein, 7 aa, Artificial Sequence
  Description of Artificial Sequence Synthetic peptide
LYRSGST

SEQ ID NO: 532 - protein, 8 aa, Artificial Sequence
  Description of Artificial Sequence Synthetic peptide
ISRTGSAT

SEQ ID NO: 533 - protein, 7 aa, Artificial Sequence
  Description of Artificial Sequence Synthetic peptide
LYRDGST

SEQ ID NO: 534 - protein, 9 aa, Artificial Sequence
  Description of Artificial Sequence Synthetic peptide
IAAGSPSTP

SEQ ID NO: 535 - protein, 7 aa, Artificial Sequence
  Description of Artificial Sequence Synthetic peptide
IYRDGST

SEQ ID NO: 536 - protein, 8 aa, Artificial Sequence
  Description of Artificial Sequence Synthetic peptide
ISRSGGST

SEQ ID NO: 537 - protein, 8 aa, Artificial Sequence
  Description of Artificial Sequence Synthetic peptide
ISRSGGST

SEQ ID NO: 538 - protein, 7 aa, Artificial Sequence
  Description of Artificial Sequence Synthetic peptide
LYRGGST

SEQ ID NO: 539 - protein, 7 aa, Artificial Sequence
  Description of Artificial Sequence Synthetic peptide
ITSLGSQ

SEQ ID NO: 540 - protein, 8 aa, Artificial Sequence
  Description of Artificial Sequence Synthetic peptide
ISDSGGSA

SEQ ID NO: 541 - protein, 8 aa, Artificial Sequence
  Description of Artificial Sequence Synthetic peptide
IGSDGENI

SEQ ID NO: 542 - protein, 8 aa, Artificial Sequence
  Description of Artificial Sequence Synthetic peptide
VSSSGAGT

SEQ ID NO: 543 - protein, 8 aa, Artificial Sequence
  Description of Artificial Sequence Synthetic peptide
ISRSGGTT

SEQ ID NO: 544 - protein, 8 aa, Artificial Sequence
  Description of Artificial Sequence Synthetic peptide
ISRGGGTT

SEQ ID NO: 545 - protein, 8 aa, Artificial Sequence
  Description of Artificial Sequence Synthetic peptide
VSSSGAGT

SEQ ID NO: 546 - protein, 7 aa, Artificial Sequence
  Description of Artificial Sequence Synthetic peptide
IARSGTT

SEQ ID NO: 547 - protein, 7 aa, Artificial Sequence
  Description of Artificial Sequence Synthetic peptide
IARSGTT

SEQ ID NO: 548 - protein, 7 aa, Artificial Sequence
  Description of Artificial Sequence Synthetic peptide
ISRGGVA

SEQ ID NO: 549 - protein, 7 aa, Artificial Sequence
  Description of Artificial Sequence Synthetic peptide
ISRVGTT

SEQ ID NO: 550 - protein, 8 aa, Artificial Sequence
  Description of Artificial Sequence Synthetic peptide
ISRDGAVP

SEQ ID NO: 551 - protein, 7 aa, Artificial Sequence
  Description of Artificial Sequence Synthetic peptide
ITSGGTT

SEQ ID NO: 552 - protein, 7 aa, Artificial Sequence
  Description of Artificial Sequence Synthetic peptide
TTRDGAP

SEQ ID NO: 553 - protein, 7 aa, Artificial Sequence
  Description of Artificial Sequence Synthetic peptide
RTNGGTT

SEQ ID NO: 554 - protein, 7 aa, Artificial Sequence
  Description of Artificial Sequence Synthetic peptide
ISSGGST

SEQ ID NO: 555 - protein, 7 aa, Artificial Sequence
  Description of Artificial Sequence Synthetic peptide
ISRGGST

SEQ ID NO: 556 - protein, 7 aa, Artificial Sequence
  Description of Artificial Sequence Synthetic peptide
ITSGGQI

SEQ ID NO: 557 - protein, 7 aa, Artificial Sequence
  Description of Artificial Sequence Synthetic peptide
ITNGGST

SEQ ID NO: 558 - protein, 7 aa, Artificial Sequence
  Description of Artificial Sequence Synthetic peptide
INRGGVA

SEQ ID NO: 559 - protein, 8 aa, Artificial Sequence
  Description of Artificial Sequence Synthetic peptide
ISRGTSTT

SEQ ID NO: 560 - protein, 8 aa, Artificial Sequence
  Description of Artificial Sequence Synthetic peptide
ISRDGAVP

SEQ ID NO: 561 - protein, 7 aa, Artificial Sequence
  Description of Artificial Sequence Synthetic peptide
SARRGTT

SEQ ID NO: 562 - protein, 8 aa, Artificial Sequence
  Description of Artificial Sequence Synthetic peptide
IRWSRGST

SEQ ID NO: 563 - protein, 7 aa, Artificial Sequence
  Description of Artificial Sequence Synthetic peptide
IARVGTT

SEQ ID NO: 564 - protein, 8 aa, Artificial Sequence
  Description of Artificial Sequence Synthetic peptide
VNAGADTT

SEQ ID NO: 565 - protein, 7 aa, Artificial Sequence
  Description of Artificial Sequence Synthetic peptide
ITSGGST

SEQ ID NO: 566 - protein, 8 aa, Artificial Sequence
  Description of Artificial Sequence Synthetic peptide
ISRSGGST

SEQ ID NO: 567 - protein, 7 aa, Artificial Sequence
  Description of Artificial Sequence Synthetic peptide
LYRSGST

SEQ ID NO: 568 - protein, 8 aa, Artificial Sequence
  Description of Artificial Sequence Synthetic peptide
VSGGGGGT

SEQ ID NO: 569 - protein, 7 aa, Artificial Sequence
  Description of Artificial Sequence Synthetic peptide
LFRSGST

SEQ ID NO: 570 - protein, 8 aa, Artificial Sequence
  Description of Artificial Sequence Synthetic peptide
ISRSGGTT

SEQ ID NO: 571 - protein, 7 aa, Artificial Sequence
  Description of Artificial Sequence Synthetic peptide
ISSGGRT

SEQ ID NO: 572 - protein, 7 aa, Artificial Sequence
  Description of Artificial Sequence Synthetic peptide
LFRSGST

SEQ ID NO: 573 - protein, 8 aa, Artificial Sequence
  Description of Artificial Sequence Synthetic peptide
ISRGGGTT

SEQ ID NO: 574 - protein, 7 aa, Artificial Sequence
  Description of Artificial Sequence Synthetic peptide
LYRSGST

SEQ ID NO: 575 - protein, 7 aa, Artificial Sequence
  Description of Artificial Sequence Synthetic peptide
LYRSGST

SEQ ID NO: 576 - protein, 7 aa, Artificial Sequence
  Description of Artificial Sequence Synthetic peptide
LYRSGST

SEQ ID NO: 577 - protein, 8 aa, Artificial Sequence
  Description of Artificial Sequence Synthetic peptide
AALLDSYY

SEQ ID NO: 578 - protein, 8 aa, Artificial Sequence
  Description of Artificial Sequence Synthetic peptide
TALLDSYY

SEQ ID NO: 579 - protein, 8 aa, Artificial Sequence
  Description of Artificial Sequence Synthetic peptide
TALLDSYY

SEQ ID NO: 580 - protein, 8 aa, Artificial Sequence
  Description of Artificial Sequence Synthetic peptide
AALLDSYY

SEQ ID NO: 581 - protein, 8 aa, Artificial Sequence
  Description of Artificial Sequence Synthetic peptide
AALLDSYY

SEQ ID NO: 582 - protein, 8 aa, Artificial Sequence
  Description of Artificial Sequence Synthetic peptide
AALLDSYY

SEQ ID NO: 583 - protein, 8 aa, Artificial Sequence
  Description of Artificial Sequence Synthetic peptide
AALLDSYY

SEQ ID NO: 584 - protein, 8 aa, Artificial Sequence
  Description of Artificial Sequence Synthetic peptide
AALLDSYY

SEQ ID NO: 585 - protein, 8 aa, Artificial Sequence
  Description of Artificial Sequence Synthetic peptide
AALLDSYY

SEQ ID NO: 586 - protein, 8 aa, Artificial Sequence
  Description of Artificial Sequence Synthetic peptide
AALLDSYY

SEQ ID NO: 587 - protein, 17 aa, Artificial Sequence
  Description of Artificial Sequence Synthetic peptide
NAAQSRTSWLFPDEYDY

SEQ ID NO: 588 - protein, 19 aa, Artificial Sequence
  Description of Artificial Sequence Synthetic peptide
AADRVTDTYYYLNPESYDY

SEQ ID NO: 589 - protein, 10 aa, Artificial Sequence
  Description of Artificial Sequence Synthetic peptide
FASLIDAGTY

SEQ ID NO: 590 - protein, 19 aa, Artificial Sequence
  Description of Artificial Sequence Synthetic peptide
AADRVTDTYYYLNPESYDY

SEQ ID NO: 591 - protein, 5 aa, Artificial Sequence
  Description of Artificial Sequence Synthetic peptide
NTATS

SEQ ID NO: 592 - protein, 10 aa, Artificial Sequence
  Description of Artificial Sequence Synthetic peptide
FASVFDAGTY

SEQ ID NO: 593 - protein, 10 aa, Artificial Sequence
  Description of Artificial Sequence Synthetic peptide
FASIFDAGTY

SEQ ID NO: 594 - protein, 10 aa, Artificial Sequence
  Description of Artificial Sequence Synthetic peptide
FASYLGAGAY

SEQ ID NO: 595 - protein, 11 aa, Artificial Sequence
  Description of Artificial Sequence Synthetic peptide
VMGAAGQGWRY

SEQ ID NO: 596 - protein, 10 aa, Artificial Sequence
  Description of Artificial Sequence Synthetic peptide
WASYLGAGTY

SEQ ID NO: 597 - protein, 6 aa, Artificial Sequence
  Description of Artificial Sequence Synthetic peptide
SKEGGL

SEQ ID NO: 598 - protein, 10 aa, Artificial Sequence
  Description of Artificial Sequence Synthetic peptide
FASVFDAGTY

SEQ ID NO: 599 - protein, 10 aa, Artificial Sequence
  Description of Artificial Sequence Synthetic peptide
FASLFDAGTY

SEQ ID NO: 600 - protein, 16 aa, Artificial Sequence
  Description of Artificial Sequence Synthetic peptide
ASDGPWRATTPDAYDY

SEQ ID NO: 601 - protein, 5 aa, Artificial Sequence
  Description of Artificial Sequence Synthetic peptide
YSNQY

SEQ ID NO: 602 - protein, 11 aa, Artificial Sequence
  Description of Artificial Sequence Synthetic peptide
ATRCTVVPGIT

SEQ ID NO: 603 - protein, 6 aa, Artificial Sequence
  Description of Artificial Sequence Synthetic peptide
WIPPIP

SEQ ID NO: 604 - protein, 11 aa, Artificial Sequence
  Description of Artificial Sequence Synthetic peptide
ATRCTVVPGIT

SEQ ID NO: 605 - protein, 21 aa, Artificial Sequence
  Description of Artificial Sequence Synthetic peptide
AARRSDFTGDYAYSGRSAYDY

SEQ ID NO: 606 - protein, 9 aa, Artificial Sequence
  Description of Artificial Sequence Synthetic peptide
RGRAGERVY

SEQ ID NO: 607 - protein, 18 aa, Artificial Sequence
  Description of Artificial Sequence Synthetic peptide
AAKYGGSLSYMHPTGYTY

SEQ ID NO: 608 - protein, 16 aa, Artificial Sequence
  Description of Artificial Sequence Synthetic peptide
AASKANMPALPANYDY

SEQ ID NO: 609 - protein, 6 aa, Artificial Sequence
  Description of Artificial Sequence Synthetic peptide
WIPPIP

SEQ ID NO: 610 - protein, 6 aa, Artificial Sequence
  Description of Artificial Sequence Synthetic peptide
WIPPIP

SEQ ID NO: 611 - protein, 17 aa, Artificial Sequence
  Description of Artificial Sequence Synthetic peptide
ATTRVWPTQHQMGQIEY

SEQ ID NO: 612 - protein, 12 aa, Artificial Sequence
  Description of Artificial Sequence Synthetic peptide
VADWQYGSTWNY

SEQ ID NO: 613 - protein, 17 aa, Artificial Sequence
  Description of Artificial Sequence Synthetic peptide
AAASSSSRLLEPIGYNY

SEQ ID NO: 614 - protein, 14 aa, Artificial Sequence
  Description of Artificial Sequence Synthetic peptide
AGNLKRSETSYYWK

SEQ ID NO: 615 - protein, 15 aa, Artificial Sequence
  Description of Artificial Sequence Synthetic peptide
TADRHEWGRLMKGDY

SEQ ID NO: 616 - protein, 20 aa, Artificial Sequence
  Description of Artificial Sequence Synthetic peptide
AATSRSLTSAMTREIRAYDY

SEQ ID NO: 617 - protein, 9 aa, Artificial Sequence
  Description of Artificial Sequence Synthetic peptide
RGRAGMEVY

SEQ ID NO: 618 - protein, 11 aa, Artificial Sequence
  Description of Artificial Sequence Synthetic peptide
ATRCTVVPGIS

SEQ ID NO: 619 - protein, 17 aa, Artificial Sequence
  Description of Artificial Sequence Synthetic peptide
AAASSSSRPLEPIGYNY

SEQ ID NO: 620 - protein, 19 aa, Artificial Sequence
  Description of Artificial Sequence Synthetic peptide
AAGNTGPFNLLHSSAQYAY

SEQ ID NO: 621 - protein, 11 aa, Artificial Sequence
  Description of Artificial Sequence Synthetic peptide
ATRCTVVPGIS

SEQ ID NO: 622 - protein, 6 aa, Artificial Sequence
  Description of Artificial Sequence Synthetic peptide
WIPPLP

SEQ ID NO: 623 - protein, 12 aa, Artificial Sequence
  Description of Artificial Sequence Synthetic peptide
AARYDMDYEYKT

SEQ ID NO: 624 - protein, 10 aa, Artificial Sequence
  Description of Artificial Sequence Synthetic peptide
FASVVDAGTY

SEQ ID NO: 625 - protein, 20 aa, Artificial Sequence
  Description of Artificial Sequence Synthetic peptide
AAQGRILRGRGLFKASDYDY

SEQ ID NO: 626 - protein, 11 aa, Artificial Sequence
  Description of Artificial Sequence Synthetic peptide
RLSRYYNSNIY

SEQ ID NO: 627 - protein, 13 aa, Artificial Sequence
  Description of Artificial Sequence Synthetic peptide
KAELYTLQHNYEY

SEQ ID NO: 628 - protein, 11 aa, Artificial Sequence
  Description of Artificial Sequence Synthetic peptide
HLSRYYNSNIY

SEQ ID NO: 629 - protein, 14 aa, Artificial Sequence
  Description of Artificial Sequence Synthetic peptide
LAEERPYYGGPLEY

SEQ ID NO: 630 - protein, 10 aa, Artificial Sequence
  Description of Artificial Sequence Synthetic peptide
FANIVDRPVS

SEQ ID NO: 631 - protein, 10 aa, Artificial Sequence
  Description of Artificial Sequence Synthetic peptide
FASVLGAGTY

SEQ ID NO: 632 - protein, 11 aa, Artificial Sequence
  Description of Artificial Sequence Synthetic peptide
RLSRYYNSNIY

SEQ ID NO: 633 - protein, 5 aa, Artificial Sequence
  Description of Artificial Sequence Synthetic peptide
NAGDY

SEQ ID NO: 634 - protein, 10 aa, Artificial Sequence
  Description of Artificial Sequence Synthetic peptide
YVDPLGRVPR

SEQ ID NO: 635 - protein, 10 aa, Artificial Sequence
  Description of Artificial Sequence Synthetic peptide
FASVLGAGTY

SEQ ID NO: 636 - protein, 7 aa, Artificial Sequence
  Description of Artificial Sequence Synthetic peptide
NAPSLGY

SEQ ID NO: 637 - protein, 10 aa, Artificial Sequence
  Description of Artificial Sequence Synthetic peptide
FASALGAGVY

SEQ ID NO: 638 - protein, 10 aa, Artificial Sequence
  Description of Artificial Sequence Synthetic peptide
FASIIDAGTY

SEQ ID NO: 639 - protein, 16 aa, Artificial Sequence
  Description of Artificial Sequence Synthetic peptide
AARSGGSYTYTGSYHY

SEQ ID NO: 640 - protein, 16 aa, Artificial Sequence
  Description of Artificial Sequence Synthetic peptide
AANRYGSSSYQGQYAS

SEQ ID NO: 641 - protein, 16 aa, Artificial Sequence
  Description of Artificial Sequence Synthetic peptide
AAQQWPDPRNPNGYDY

SEQ ID NO: 642 - protein, 16 aa, Artificial Sequence
  Description of Artificial Sequence Synthetic peptide
AAAKAGHWGRDANYDY

SEQ ID NO: 643 - protein, 16 aa, Artificial Sequence
  Description of Artificial Sequence Synthetic peptide
AAAKAGQWGRDAKYDY

SEQ ID NO: 644 - protein, 21 aa, Artificial Sequence
  Description of Artificial Sequence Synthetic peptide
TADLKGLWALGLPGHYASWDS

SEQ ID NO: 645 - protein, 9 aa, Artificial Sequence
  Description of Artificial Sequence Synthetic peptide
RARRGWAIY

SEQ ID NO: 646 - protein, 16 aa, Artificial Sequence
  Description of Artificial Sequence Synthetic peptide
AADSGGSWGRGTTYDY

SEQ ID NO: 647 - protein, 17 aa, Artificial Sequence
  Description of Artificial Sequence Synthetic peptide
AAYNTAGWGEPHQSYRY

SEQ ID NO: 648 - protein, 15 aa, Artificial Sequence
  Description of Artificial Sequence Synthetic peptide
AGRRSVVVRSFDYDY

SEQ ID NO: 649 - protein, 9 aa, Artificial Sequence
  Description of Artificial Sequence Synthetic peptide
NVNWALHDS

SEQ ID NO: 650 - protein, 16 aa, Artificial Sequence
  Description of Artificial Sequence Synthetic peptide
AANSGGHWWRGATYDY

SEQ ID NO: 651 - protein, 9 aa, Artificial Sequence
  Description of Artificial Sequence Synthetic peptide
NVNWALHDS

SEQ ID NO: 652 - protein, 17 aa, Artificial Sequence
  Description of Artificial Sequence Synthetic peptide
AAYNTANWGQPHQSYRH

SEQ ID NO: 653 - protein, 12 aa, Artificial Sequence
  Description of Artificial Sequence Synthetic peptide
NVVTYGSNRRDF

SEQ ID NO: 654 - protein, 16 aa, Artificial Sequence
  Description of Artificial Sequence Synthetic peptide
AANRYGSSSYQGQYGS

SEQ ID NO: 655 - protein, 16 aa, Artificial Sequence
  Description of Artificial Sequence Synthetic peptide
AANRYGSSSYQGQYDY

SEQ ID NO: 656 - protein, 9 aa, Artificial Sequence
  Description of Artificial Sequence Synthetic peptide
NVNWALQDS

SEQ ID NO: 657 - protein, 9 aa, Artificial Sequence
  Description of Artificial Sequence Synthetic peptide
RARRGWAIY

SEQ ID NO: 658 - protein, 21 aa, Artificial Sequence
  Description of Artificial Sequence Synthetic peptide
AADLTGLWALGLPGHYASWDS

SEQ ID NO: 659 - protein, 10 aa, Artificial Sequence
  Description of Artificial Sequence Synthetic peptide
QLGRTVLDYF

SEQ ID NO: 660 - protein, 16 aa, Artificial Sequence
  Description of Artificial Sequence Synthetic peptide
AAAKAGQWGRYANYDY

SEQ ID NO: 661 - protein, 16 aa, Artificial Sequence
  Description of Artificial Sequence Synthetic peptide
AANPYGSSSYQGQYGS

SEQ ID NO: 662 - protein, 16 aa, Artificial Sequence
  Description of Artificial Sequence Synthetic peptide
AANRYGSSSYQGQYGS

SEQ ID NO: 663 - protein, 16 aa, Artificial Sequence
  Description of Artificial Sequence Synthetic peptide
AAAKAGHWGRDANYDY

SEQ ID NO: 664 - protein, 10 aa, Artificial Sequence
  Description of Artificial Sequence Synthetic peptide
YVDRRGAVPT

SEQ ID NO: 665 - protein, 10 aa, Artificial Sequence
  Description of Artificial Sequence Synthetic peptide
YVDRRDVVPT

SEQ ID NO: 666 - protein, 10 aa, Artificial Sequence
  Description of Artificial Sequence Synthetic peptide
YADRFSGSPT

SEQ ID NO: 667 - protein, 10 aa, Artificial Sequence
  Description of Artificial Sequence Synthetic peptide
FANVISGPVY

SEQ ID NO: 668 - protein, 16 aa, Artificial Sequence
  Description of Artificial Sequence Synthetic peptide
AASRQGNPYAQTSYDY

SEQ ID NO: 669 - protein, 14 aa, Artificial Sequence
  Description of Artificial Sequence Synthetic peptide
NALVSRRDSAAYFA

SEQ ID NO: 670 - protein, 9 aa, Artificial Sequence
  Description of Artificial Sequence Synthetic peptide
KARKDSHDY

SEQ ID NO: 671 - protein, 13 aa, Artificial Sequence
  Description of Artificial Sequence Synthetic peptide
NAHIREYYSTYDY

SEQ ID NO: 672 - protein, 14 aa, Artificial Sequence
  Description of Artificial Sequence Synthetic peptide
NALFNPIDGPARYY

SEQ ID NO: 673 - protein, 14 aa, Artificial Sequence
  Description of Artificial Sequence Synthetic peptide
TALFNPVDGTARYY

SEQ ID NO: 674 - protein, 14 aa, Artificial Sequence
  Description of Artificial Sequence Synthetic peptide
NAASSTWPPRDYDY

SEQ ID NO: 675 - protein, 14 aa, Artificial Sequence
  Description of Artificial Sequence Synthetic peptide
NADERPYYGDSVLS

SEQ ID NO: 676 - protein, 10 aa, Artificial Sequence
  Description of Artificial Sequence Synthetic peptide
YADRFSGSPT

SEQ ID NO: 677 - protein, 18 aa, Artificial Sequence
  Description of Artificial Sequence Synthetic peptide
AADADRSTTIYSRDIYDY

SEQ ID NO: 678 - protein, 16 aa, Artificial Sequence
  Description of Artificial Sequence Synthetic peptide
AASRQGLPYVETSYDY

SEQ ID NO: 679 - protein, 10 aa, Artificial Sequence
  Description of Artificial Sequence Synthetic peptide
YVDRRDEVPT

SEQ ID NO: 680 - protein, 18 aa, Artificial Sequence
  Description of Artificial Sequence Synthetic peptide
AADGNPAKLVLDQYGMDY

SEQ ID NO: 681 - protein, 10 aa, Artificial Sequence
  Description of Artificial Sequence Synthetic peptide
YVDQRGVVPT

SEQ ID NO: 682 - protein, 17 aa, Artificial Sequence
  Description of Artificial Sequence Synthetic peptide
AAYNTAGWGEPHQSYRY

SEQ ID NO: 683 - protein, 15 aa, Artificial Sequence
  Description of Artificial Sequence Synthetic peptide
NAEVHVWGVPGPRDY

SEQ ID NO: 684 - protein, 16 aa, Artificial Sequence
  Description of Artificial Sequence Synthetic peptide
AANPYGSSSYQGQYAS

SEQ ID NO: 685 - protein, 9 aa, Artificial Sequence
  Description of Artificial Sequence Synthetic peptide
NVNWALHDS

SEQ ID NO: 686 - protein, 16 aa, Artificial Sequence
  Description of Artificial Sequence Synthetic peptide
AAATAGHWGRDANYDY

SEQ ID NO: 687 - protein, 9 aa, Artificial Sequence
  Description of Artificial Sequence Synthetic peptide
NVNWALHDS

SEQ ID NO: 688 - protein, 16 aa, Artificial Sequence
  Description of Artificial Sequence Synthetic peptide
AANPYGSSSYQGQYGS

SEQ ID NO: 689 - protein, 9 aa, Artificial Sequence
  Description of Artificial Sequence Synthetic peptide
TMVWGLRYY

SEQ ID NO: 690 - protein, 9 aa, Artificial Sequence
  Description of Artificial Sequence Synthetic peptide
NVNWALHDS

SEQ ID NO: 691 - protein, 16 aa, Artificial Sequence
  Description of Artificial Sequence Synthetic peptide
AANPYGSSSYQGQYGS

SEQ ID NO: 692 - protein, 9 aa, Artificial Sequence
  Description of Artificial Sequence Synthetic peptide
NVNWALHDS

SEQ ID NO: 693 - protein, 9 aa, Artificial Sequence
  Description of Artificial Sequence Synthetic peptide
NVNWALHDS

SEQ ID NO: 694 - protein, 9 aa, Artificial Sequence
  Description of Artificial Sequence Synthetic peptide
NVNWALHDS

SEQ ID NO: 695 - protein, 6 aa, Artificial Sequence
  Description of Artificial Sequence Synthetic 6xHis tag
HHHHHH

(19) United States
(12) Patent Application Publication
(10) Pub. No.: US 2021/0269512 A1
(43) Pub. Date: Sep. 2, 2021

(54) ANTIBODIES AGAINST DISEASE CAUSING AGENTS OF POULTRY AND USES THEREOF

(71) Applicant: NOVOBIND LIVESTOCK THERAPEUTICS INC., Vancouver (CA)

(72) Inventors: Hamlet ABNOUSI, Vancouver (CA); Slade Andrew LOUTET, Vancouver (CA); Filip VAN PETEGEM, Vancouver (CA); Tsz Ying Silvia CHEUNG, Vancouver (CA)

(21) Appl. No.: 17/141,052

(22) Filed: Jan. 4, 2021

Related U.S. Application Data
(63) Continuation of application No. PCT/IB2019/001198, filed on Jul. 3, 2019.
(60) Provisional application No. 62/694,164, filed on Jul. 5, 2018.

(51) Int. Cl. C07K 16/12

(52) U.S. Cl. CPC C07K 16/1282; C07K 2317/22; C07K 2317/76

(57) ABSTRACT

Described herein are methods and antibodies useful for reducing, eliminating, or preventing infection with a bacterial population in an animal. Also described herein are antigens useful for targeting by heavy chain antibodies and VHH fragments for reducing a bacterial population in an animal.

### CROSS-REFERENCE

This application claims the benefit of U.S. Provisional Application No. 62/694,164, filed Jul. 5, 2018, which application is incorporated herein by reference. Priority is claimed pursuant to 35 U.S.C. § 119. The above noted patent application is incorporated by reference as if set forth fully herein.

### SEQUENCE LISTING

The instant application contains a Sequence Listing which has been submitted electronically in ASCII format and is hereby incorporated by reference in its entirety. Said ASCII copy, created on Feb. 5, 2020, is named 48647_706_301_SL.txt and is 344,000 bytes in size.

### FIELD OF THE INVENTION

This invention relates to methods and compositions for the control of microorganisms associated with necrotic enteritis and uses thereof.

### BACKGROUND OF THE INVENTION

Losses to the agriculture industry following contamination of livestock with pathogens are a global burden. With a growing global population and no significant increase in the amount of farmland available to agriculture, there is a need to produce larger quantities of food without using more space. Traditional treatment of animals with antibiotics is a major contributor to the emergence of multi-drug resistant organisms and is widely recognised as an unsustainable solution to controlling contamination of livestock. There is a need for the development of pathogen-specific molecules that inhibit infection or association of the pathogen with the host, without encouraging resistance. Global losses to the poultry industry due to the pathogenic organisms that cause necrotic enteritis has been estimated to be $6 billion⁽¹⁾ USD per annum. The bacterium Clostridium perfringens is the causative agent of necrotic enteritis in poultry in conjunction with a variety of predisposing factors⁽²⁾.

### SUMMARY OF THE INVENTION

With reference to the definitions set out below, described herein are polypeptides comprising heavy chain variable region fragments (V_{H}Hs) whose intended use includes but is not limited to the following applications in agriculture or an unrelated field: diagnostics, in vitro assays, feed, therapeutics, substrate identification, nutritional supplementation, bioscientific and medical research, and companion diagnostics. Also described herein are polypeptides comprising V_{H}Hs that bind and decrease the virulence of disease-causing agents in agriculture. Further to these descriptions, set out below are the uses of polypeptides that comprise V_{H}Hs in methods of reducing transmission and severity of disease in host animals, including their use as an ingredient in a product. Further described are the means to produce, characterise, refine and modify V_{H}Hs for this purpose.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-1B: Panel A shows a schematic of camelid heavy chain only antibodies and their relationship to V_{H}H domains. Panel B illustrates the framework regions (FRs) and complementarity determining regions (CDRs) of the V_{H}H domain.

FIGS. 2A-2F: Shows phage ELISA binding data for V_{H}H antibodies of this disclosure.

FIG. 3: Shows that unlabeled CnaA can outcompete labeled CnaA for collagen binding

### DEFINITIONS

In describing the present invention, the following terminology is used in accordance with the definitions below.

In the following description, certain specific details are set forth in order to provide a thorough understanding of various embodiments. However, one skilled in the art will understand that the embodiments provided may be practiced without these details. Unless the context requires otherwise, throughout the specification and claims which follow, the word “comprise” and variations thereof, such as, “comprises” and “comprising” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed embodiments.

### 1) Host

As referred to herein, “host”, “host organism”, “recipient animal”, “host animal” and variations thereof refer to the intended recipient of the product when the product constitutes a feed. In certain embodiments, the host is from the superorder Galloanserae. In certain embodiments, the host is a poultry animal. In certain embodiments, the poultry animal is a chicken, turkey, duck, quail, pigeon, squab or goose. In certain embodiments, the poultry animal is a chicken.

### 2) Pathogens

As referred to herein, “pathogen”, “pathogenic”, and variations thereof refer to virulent microorganisms, that can be associated with host organisms, that give rise to a symptom or set of symptoms in that organism that are not present in uninfected host organisms, including the reduction in ability to survive, thrive, reproduce. Without limitation, pathogens encompass parasites, bacteria, viruses, prions, protists, fungi and algae. In certain embodiments, the pathogen is a bacterium belonging to the Clostridium genus.

“Virulence”, “virulent” and variations thereof refer to a pathogen's ability to cause symptoms in a host organism. “Virulence factor” refers to nucleic acids, plasmids, genomic islands, genes, peptides, proteins, toxins, lipids, macromolecular machineries or complexes thereof that have a demonstrated or putative role in infection.

“Disease-causing agent” refers to a microorganism, pathogen or virulence factor with a demonstrated or putative role in infection.

### 3) Bacteria

As referred to herein, “bacteria”, “bacterial” and variations thereof refer, without limitation, to Clostridium species, or any other bacterial species associated with host organisms. In certain embodiments, bacteria may not be virulent in all host organisms it is associated with.

### 4) Antibodies

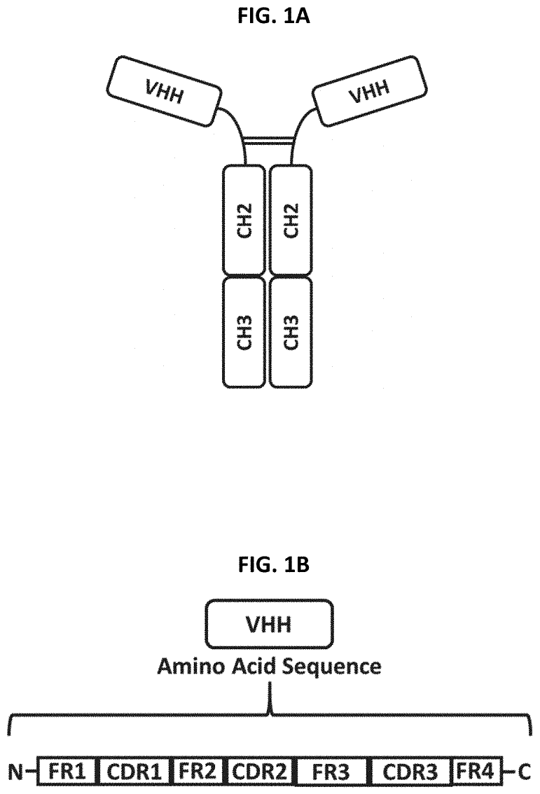

A schematic of camelid heavy chain only antibodies and their relationship to V_{H}H domains and complementarity determining regions (CDRs) is shown in FIG. 1. (Panel A). A camelid heavy chain only antibody consists of two heavy chains linked by a disulphide bridge. Each heavy chain contains two constant immunoglobulin domains (CH2 and CH3) linked through a hinge region to a variable immunoglobulin domain (V_{H}H). (Panel B) are derived from single V_{H}H domains. Each V_{H}H domain contains an amino acid sequence of approximately 110-130 amino acids. The V_{H}H domain consists of the following regions starting at the N-terminus (N): framework region 1 (FR1), complementarity-determining region 1 (CDR1), framework region 2 (FR2), complementarity-determining region 2 (CDR2), framework region 3 (FR3), complementarity-determining region 3 (CDR3), and framework region 4 (FR4). The domain ends at the C-terminus (C). The complementarity-determining regions are highly variable, determine antigen binding by the antibody, and are held together in a scaffold by the framework regions of the V_{H}H domain. The framework regions consist of more conserved amino acid sequences; however, some variability exists in these regions.

As referred to herein “V_{H}H” refers to an antibody or antibody fragment comprising a single heavy chain variable region which may be derived from natural or synthetic sources. NBXs referred to herein are an example of a V_{H}H. In a certain aspect a V_{H}H may lack a portion of a heavy chain constant region (CH2 or CH3), or an entire heavy chain constant region.

As referred to herein “heavy chain antibody” refers to an antibody that comprises two heavy chains and lacks the two light chains normally found in a conventional antibody. The heavy chain antibody may originate from a species of the Camelidae family or Chondrichthyes class. Heavy chain antibodies retain specific binding to an antigen in the absence of any light chain.

As referred to herein “specific binding”, “specifically binds” or variations thereof refer to binding that occurs between an antibody and its target molecule that is mediated by at least one complementarity determining region (CDR) of the antibody's variable region. Binding that is between the constant region and another molecule, such as Protein A or G, for example, does not constitute specific binding.

As referred to herein “antibody fragment” refers to any portion of a conventional or heavy chain antibody that retains a capacity to specifically bind a target antigen and may include a single chain antibody, a variable region fragment of a heavy chain antibody, a nanobody, a polypeptide or an immunoglobulin new antigen receptor (IgNAR).

As referred to herein an “antibody originates from a species” when any of the CDR regions of the antibody were raised in an animal of said species. Antibodies that are raised in a certain species and then optimized by an in vitro method (e.g., phage display) are considered to have originated from that species.

As referred to herein “conventional antibody” refers to any full-sized immunoglobulin that comprises two heavy chain molecules and two light chain molecules joined together by a disulfide bond. In certain embodiments, the antibodies, compositions, feeds, products, and methods described herein do not utilize conventional antibodies.

### 5) Production System

As referred to herein, “production system” and variations thereof refer to any system that can be used to produce any physical embodiment of the invention or modified forms of the invention. Without limitation, this includes but is not limited to biological production by any of the following: bacteria, yeast, algae, arthropods, arthropod cells, plants, mammalian cells. Without limitation, biological production can give rise to antibodies that can be intracellular, periplasmic, membrane-associated, secreted, or phage-associated. Without limitation, “production system” and variations thereof also include, without limitation, any synthetic production system. This includes, without limitation, de novo protein synthesis, protein synthesis in the presence of cell extracts, protein synthesis in the presence of purified enzymes, and any other alternative protein synthesis system.

### 6) Product

As referred to herein, “product” refers to any physical embodiment of the invention or modified forms of the invention, wherein the binding of the V_{H}H to any molecule, including itself, defines its use. Without limitation, this includes a feed, a feed additive, a nutritional supplement, a premix, a medicine, a therapeutic, a drug, a diagnostic tool, a component or entirety of an in vitro assay, a component or the entirety of a diagnostic assay (including companion diagnostic assays).

### 7) Feed Product

As referred to herein, “feed product” refers to any physical embodiment of the invention or modified forms of the invention, wherein the binding of the V_{H}H to any molecule, including itself, defines its intended use as a product that is taken up by a host organism. Without limitation, this includes a feed, a pellet, a feed additive, a nutritional supplement, a premix, a medicine, a therapeutic or a drug.

### DETAILED DESCRIPTION OF THE INVENTION

Descriptions of the invention provided are to be interpreted in conjunction with the definitions and caveats provided herein.

For many years, the agriculture industry has utilized antibiotics to control pathogenic bacteria. These antibiotics also acted as growth promoters. This approach has contributed greatly to the spread of antibiotic resistance amongst pathogenic organisms. To phase out antibiotics for non-medicinal purposes and limit antimicrobial resistance, the use of antibiotics as growth promoters in animal feed has already been banned in Europe (effective from 2006). Widespread protection of farmed animals through vaccination has failed due to the short lifespan of many agriculturally important animals, logistical challenges with vaccination of industrial-sized flocks, and high costs. The withdrawal of prophylactic antibiotics in animal feed and the failure of vaccination to offer widespread protection underpins the need for the development of non-antibiotic products to administer to agricultural animals to prevent infection and promote growth.

Significant pathogens affecting poultry animals include bacteria, such as members of the Clostridium and Salmonella genera, among others, as well as parasites, such as members of the Eimeria genus.

Losses due to Clostridium perfringens, the causative agent of necrotic enteritis are estimated at $6 billion⁽¹⁾ USD per annum. Necrotic enteritis can lead to significant mortality in chicken flocks⁽³⁾. At subclinical levels, damage to the intestinal mucosa caused by C. perfringens leads to decreased digestion and absorption, reduced weight gain and increased feed conversion ratio⁽³⁾. Typically, necrotic enteritis occurs after some other predisposing factor causes mucosal damage to the chicken⁽²⁾ C. perfringens virulence factors associated with necrotic enteritis have been shown to include production of toxins and adherence to collagen⁽⁴⁾.

Subclinical infection by Eimeria parasites is one of the most common predisposing factors for necrotic enteritis⁽²⁾. These parasites can physically damage the epithelial layer and induce mucose generation⁽⁵⁾. In addition, Eimeria parasites are also the causative agent of coccidiosis in chickens, a disease that is estimated to cause €10 billion in poultry losses globally⁽⁶⁾. Coccidiosis is characterized by reduced weight gain and feed conversion, malabsorption, cell lysis of cells linking, and diarrhea⁽⁷⁾.

Changes to the gastrointestinal tract microbiota can also serve to induce necrotic enteritis. For example, early infections early of chicks by Salmonella enterica can result in the development of necrotic enteritis in experimental models, possibly through alteration of the host immune response⁽⁸⁾.

Other proposed predisposing factors for the development of necrotic enteritis include immune suppression by viral infections, physical changes to the gut caused by alterations to the diet, and poor animal husbandry⁽²⁾.

Earlier efforts in the field of this invention rely on the host organism to generate protection against disease-causing agents. This approach is often limited by the short lifespan of the host organisms affected by the pathogens listed above, which do allow the host organism's immune system enough time to generate long-lasting immunity. Furthermore, the effectiveness of prior arts is limited by technical challenges associated with widespread vaccination of large flocks of host organisms. These problems are circumvented by introducing exogenous peptides that neutralise the virulence and spread of the disease-causing agent into the host via feed without eliciting the host immune response. Moreover, the methods described herein provide scope for the adaptation and refinement of neutralising peptides, which provides synthetic functionality beyond what the host is naturally able to produce.

Antibody heavy chain variable region fragments (V_{H}Hs) are small (12-15 kDa) proteins that comprise specific binding regions to antigens. When introduced into an animal, V_{H}Hs bind and neutralise the effect of disease-causing agents in situ. Owing to their smaller mass, they are less susceptible than conventional antibodies, such as previously documented IgYs, to cleavage by enzymes found in host organisms, more resilient to temperature and pH changes, more soluble, have low systemic absorption and are easier to recombinantly produce on a large scale, making them more suitable for use in animal therapeutics than conventional antibodies.

### Antibodies for Preventing or Reducing Virulence (Summary)

In one aspect, the present invention provides a polypeptide or pluralities thereof comprising a V_{H}H or V_{H}Hs that bind disease-causing agents to reduce the severity and transmission of disease between and across species. In certain embodiments, the V_{H}H is supplied to host animals. In certain embodiments, the V_{H}H is an ingredient of a product.

### Binding to Reduce Virulence

In another aspect, the present invention provides a polypeptide or pluralities thereof comprising a V_{H}H or V_{H}Hs that bind disease-causing agents, and in doing so, reduce the ability of the disease-causing agent to exert a pathological function or contribute to a disease phenotype. In certain embodiments, binding of the V_{H}H(s) to the disease-causing agent reduces the rate of replication of the disease-causing agent. In certain embodiments, binding of the V_{H}H(s) to the disease-causing agent reduces the ability of the disease-causing agent to bind to its cognate receptor. In certain embodiments, binding of the V_{H}H(s) to the disease-causing agent reduces the ability of the disease-causing agent to interact with another molecule or molecules. In certain embodiments, binding of the V_{H}H(s) to the disease-causing agent reduces the mobility or motility of the disease-causing agent. In certain embodiments, binding of the V_{H}H(s) to the disease-causing agent reduces the ability of the disease-causing agent to reach the site of infection. In certain embodiments, binding of the V_{H}H(s) to the disease-causing agent reduces the ability of the disease-causing agent to cause cell death.

Antibodies Derived from Llamas

In a further aspect, the present invention provides a method for the inoculation of Camelid or other species with recombinant virulence factors, the retrieval of mRNA encoding V_{H}H domains from lymphocytes of the inoculated organism, the reverse transcription of mRNA encoding V_{H}H domains to produce cDNA, the cloning of cDNA into a suitable vector and the recombinant expression of the V_{H}H from the vector. In certain embodiments, the camelid can be a dromedary, camel, llama, alpaca, vicuna or guacano, without limitation. In certain embodiments, the inoculated species can be, without limitation, any organism that can produce single domain antibodies, including cartilaginous fish, such as a member of the Chondrichthyes class of organisms, which includes for example sharks, rays, skates and sawfish. In certain embodiments, the heavy chain antibody comprises a sequence set forth in Table 1. In certain embodiments, the heavy chain antibody comprises an amino acid sequence with at least 80%, 90%, 95%, 97%, or 99% identity to any sequence disclosed in Table 1. In certain embodiments, the heavy chain antibody possess a CDR1 set forth in Table 2. In certain embodiments, the heavy chain antibody possess a CDR2 set forth in Table 2. In certain embodiments, the heavy chain antibody possess a CDR3 set forth in Table 2.

TABLE 1
Unique SEQ IDs for V_{H}H antibodies of this disclosure

| SEQ | | | |
|---|---|---|---|
| ID | | | |
| NO: | NBX | Amino acid sequence | Antigen |
| 1 | NBX0301 | QVQLQESGGGVVQAGGSLSLSCSPYQRASSLFAMGWFRQSPGKEREFVAGI | NetB |
| | | SWNGDKSQYADSVKDRFTISRDNDKNTVFLQMNSLKPEDTAVYYCAAHRAS | |
| | | FELGFATHDYDFWGQGTQVTVSS | |
| 2 | NBX0302 | QVQLQESGGGLVQTGGSLRLSCVASGSIFSISSAVWSRQAPGKQREWVASIFS | NetB |
| | | DGSTNYATSVKGRFTISRDHAKNTVYLQMNSLKPEDTGVYYCAVDGYRGQGT | |
| | | QVTVSS | |
| 3 | NBX0303 | QVQLQESGGGLVQAGGSLRLSCTASGRTLSYWTMGWFRQAPGKEREFVAAI | NetB |
| | | NWSSGTRYSDSVRDRFTIDGDTDKTTVYLEMNKMNLDDSAVYYCAAHRASF | |
| | | GLGYQTHEYDFWGQGTQVTVSS | |
| 4 | NBX0304 | QVQLQESGGGLVQTGDSLRLSCTASGGTFSSYTMGWYRQAPGKGREFVGSI | NetB |
| | | TWNSEVTYYADSVKGRFTISRDNAKNMMNLQMNSLKPEDTAVYYCAAGRA | |
| | | GSGFTSWGQGTQVTVSS | |
| 5 | NBX0305 | QVQLQESGGGLVQPGGSLRLSCTASGFTLDKYAVGWFRQAPGKEREGVSCIS | NetB |
| | | SIDDSTDYVDSVKGRFTISRDNAKNAVYLQMNSLKPEDTAVYNCMTIPLPYGS | |
| | | TCDIPSRSDLLAINYWGKGTLVTVSS | |
| 6 | NBX0306 | QVQLQQSGGGLVQPGGSLRLSCTASGFTVPYYYIGWFRQAPGKEREGISCIAS | NetB |
| | | SSGKAYYADSVKGRFTLSKDNAKNTAYLQMDSLKPEDTAVYYCAALRKYGSTC | |
| | | YLHVLEYDYWGQGTQVNVSS | |
| 7 | NBX0307 | QVQLQESGGGLVQAGGSLRLSCTASGRTLSYWTMGWFRQVPGKEREFVAAI | NetB |
| | | NWSSGTRYSESVRDRFTIDGDTDKTTVYLEMNKMNLDDSAVYYCAAHRASF | |
| | | GLGYQTHEYDFWGQGTQVTVSS | |
| 8 | NBX0308 | QVQLQQSGGGLVQAGGSLRLSCTASGRTLSYWTMGWFRQVPGKEREFVAA | NetB |
| | | INWSSGTRYSESVRDRFTIDGDTDKTTVYLEMNKMNLDDSAVYYCAAHRASF | |
| | | GLGYQTHEYDFWGQGTQVTVSS | |
| 9 | NBX0309 | QVQLQQSGGGLVQAGGSLRLSCAASGSTFNNYMIGWFRQAPGKEREFVATI | NetB |
| | | SGSGAGTFYADSVRGRFTISRDNAKNTVYLQMNSLKLEDTAGYYCARRMSRS | |
| | | GIFGLRDYDSWGQGTQVTVSS | |
| 10 | NBX0310 | QVQLQQSGGGVVQAGGSLSLSCSPYQRASSLFAMGWFRQSPGKEREFVAGI | NetB |
| | | SWNGDKSQYADSVKDRFTISRDNDKNTVFLQMNSLKPEDTAVYYCAAHRAS | |
| | | FELGFATHDYDFWGQGTQVTVSS | |
| 11 | NBX0311 | QVQLQESGGGLVQAGGSLRLSCAASGRTFSNADMAWFRQSPGKERESVAAI | NetB |
| | | SWSGGRTYYADSVKGRATISRDIAKDTVYLQMNSLKPEDTAVYYCAAGGYSN | |
| | | LPTSYGYWGQGTQVTVSS | |
| 12 | NBX0316 | QVQLQESGGGLVQTGGSLRLSCAASGRAFSTYGMGWFRQAPGKEREFVAGI | CnaA |
| | | SSSGAGSAYVDSVKHRFTVSRDNAKNTMYLQMNSLKPEDTAVYYCAASTTS | |
| | | WGKFAHYIYWGQGTQVTVSS | |
| 13 | NBX0317 | QVQLQESGGGLVQAGGSLRLSCAASGGTFSSYIMGWFRQAPGKDREFVGAI | CnaA |
| | | SWSGGVTHYADSVKGRFTISRDNAKNTVYLQMNSLKPEDTAVYYCAADSRIS | |
| | | AGGSYYEADFGSWGQGTQVTVSS | |
| 14 | NBX0318 | QVQLQESGGGLVQAGGSLRLSCVVSGSIMSIRVMGWYRQAPGKQRELVAT | NetB |
| | | MSRGNTINYADSVRGRFTISRDNAKSTVYLQMNSLKPEDTDVYYCAALLDSYY | |
| | | WGQGTQVTVSS | |
| 15 | NBX0319 | QVQLQESGGGLVQAGGSLRLSCAASASIISIRVMGWYRQAPGKQRELVATM | NetB |
| | | SRGGTINYADSVRGRFTISRDNAKSTVFLEMNSLKPEDTAVYYCTALLDSYYW | |
| | | GQGTQVTVSS | |
| 16 | NBX0320 | QVQLQESGGGLVQAGGSLRLSCAASGSIFSIRVMGWYRQAPGKQRELVATM | NetB |
| | | SRGGTINYADSVRGRFTISRDNAKITVYLQMTSLKPEDTAVYYCAALLDSYYW | |
| | | GQGTQVTVSS | |
| 17 | NBX0321 | QVQLQESGGGLVQPGGSLRLSCTASGFTLDKYAVGWFRQAPGKEREGVSCIS | NetB |
| | | SIDDSTDYVDSVKGRFTISRDNAKNAVYLQMNSLKPEDTAVYDCMTIPLPYGS | |
| | | TCRIPSRSDLLAINYWGKGTLVTVSS | |
| 18 | NBX0322 | QVQLQESGGGLVQAGGSLRLSCQGSGRTFSTYAMGWYRQAPGKEREFVAAI | NetB |
| | | TRGGNTIYADSVKGRFTISRVSDKNTVYLQMSSLKPEDTAVYYCAADRIIVPRD | |
| | | PMDYWGKGTLVTVSS | |
| 19 | NBX0323 | QVQLQQSGGGLVQAGGSLRLSCTASGRTLSYWTMGWFRQAPGKEREFVAA | NetB |
| | | INWSSGTRYSDSVRDRFTIDGDTDKTTVYLEMNKMNLDDSAVYYCAAHRASF | |
| | | GLGYQTHEYDFWGQGTQVTVSS | |
| 20 | NBX0324 | QVQLQESGGGLVQAGGSLRLTCTASGRTLSYWTMGWFRQAPGKEREFVAAI | NetB |
| | | NWSSGTRYSDSVKDRFTIDGDSDKTTVYLQMNSLNLDDSAVYYCAAHRASFG | |
| | | LGYQTHEYDFWGQGTQVTVSS | |
| 21 | NBX0325 | QVQLQESGGGLVQAGDSLRLSCLASGGTFSSYIMGWFRQAPGKDREFVGAIS | CnaA |
| | | WSGGVTHYADSVKGRFTISRDNAKNTVYLQMNSLKPEDTAVYYCAADSRISA | |
| | | GGSYYEADFGSWGQGTQVTVSS | |
| 22 | NBX0326 | QVQLQESGGGLVQAGGSLRLTCAVSGRTFSAIHMGWFRQAPGKEREFVAGI | CnaA |
| | | SWSGGGTAYGGTVKGRFTISRDNAKNTVSLQMNSLKSEDTAVYYCAASDTD | |
| | | WGRSASYDYWGQGTQVTVSA | |
| 23 | NBX0327 | QVQLQQSGGGLVQAGGSLRLSCAASGGTFSSYVMGWFRQAPGKDREFVGA | CnaA |
| | | ISWSGGVTHYADSVKGRFTISRDNAKNTVYLQMNSLKPEDTAVYYCAADSRIS | |
| | | AGGSYYEADFGSWGQGTQVTVSS | |
| 24 | NBX0328 | QVQLQQSGGGLVQAGDSLRLSCATSGRTFSSYTMGWFRQTPGKEREFVAAI | Cpa |
| | | SWSGTYYTDSVKGRFTISRDTAKNTVYLQMNSLKPEDTAVYYCAVGSRRLYYS | |
| | | SDINYWGQGTQVTVSS | |
| 25 | NBX0329 | QVQLQESGGGLVQAGGSLRLSCATSGLTVSRYTMGWFRQTPGKDREFVAAI | Cpa |
| | | SWSGTYYTDSVKGRFTISVDNAKNMVYLQMNSLKPEDTAVYYCAAGSRRLYY | |
| | | SNDINYWGQGTQVTVSS | |
| 26 | NBX0330 | QVQLQESGGGLVQAGGSLRLSCAASSRTFSNYAMAWFRQTPGKEREFLATIN | Cpa |
| | | GDTTFTIYADSVKGRFTISRDNAKNTLYLQMNSLKAEDTAVYYCAARQWNPT | |
| | | MRERDYGYWGQGTEVTVSS | |
| 27 | NBX0331 | QVQLQESGGGLVQAGGSLRLSCAASGRVFENYFMGWFRQAPGKEREFVAA | Cpb2 |
| | | TNWNTATNWNTYYAAFVKARFTISRDKAKNTLYLQMNSLKPEDTAVYYCAA | |
| | | TGSRTYDVVDYYDYWGQGTQVTVSS | |
| 28 | NBX0332 | QVQLQESGGGLVQAGGSLRLSCAASGRTFSSYSMAWFRQAPGKERESVAAIT | Cpb2 |
| | | YSGITTAYTDSVKGRFTIWRDNAKNTVYLQMNSLKPEDTAVYYCAASYSASRS | |
| | | YPFGEYDYWGQGTQVTVSS | |
| 29 | NBX0333 | QVQLQESGGGLVQAGGSLRLSCAASGRTFSSYSMAWFRQAPGKERESVAAIT | Cpb2 |
| | | YSGISTAYTDSVKGRFTISRDNAKNTVYLYMNSLKPEDTAVYYCAASYSASRSY | |
| | | PFGEYDYWGQGTQVTVSS | |
| 30 | NBX0334 | QVQLQESGGGLVQPGGSLRLSCAASGFTFSNSAMSWMRQAPGKAVEWVSS | Cpb2 |
| | | INIGGDSRRYAESVAGRFTISRDNAKNTLYLQMNSLKPEDTAVYYCAKGLASTI | |
| | | RGQGTQVTVSS | |
| 31 | NBX0335 | QVQLQESGGGLVQPGGSLRLSCAASGFTFSNSAMSWMRQAPGKGVEWVS | Cpb2 |
| | | SIEVGGGRRYAESVAGRFTISRDNAKNTLYLQMNSLKPEDTAVYYCSKGLASTI | |
| | | RGQGTQVTVSS | |
| 32 | NBX0336 | QVQLQESGGGLVQPGGSLRLSCAASGFTFSNSAMSWMRQAPGKGVEWVS | Cpb2 |
| | | SIGIDGGRRYAEAVAGRFTISRDNAKNTLYLQMNSLKPEDTAVYYCAKGLASTI | |
| | | RGQGTQVTVSS | |
| 33 | NBX0337 | QVQLQESGGGLVQPGGSLRLSCAASGFTFSNSAMSWMRQAPGKGVEWVS | Cpb2 |
| | | SIGIGGGTTRYADSVAGRFTISRDNAKNTLYLQMNSLKPEDTAVYYCAKGLAST | |
| | | IRGQGTQVTVSS | |
| 34 | NBX0338 | QVQLQESGGGLVQAGDSLRLSCATSGRSFSSYTMGWFRQTPGKEREFVAAIS | Cpa |
| | | WSGTYYTDSVKGRFTISRDNAKNTVYLQMNSLKPEDTAVYYCAVGSRRLYYSS | |
| | | DINYWGQGTQVTVSS | |
| 35 | NBX0339 | QVQLQESGGGLVQAGDSLRLSCATSGLTVSRYTMGWFRQTPGKEREFVAAIS | Cpa |
| | | WSGTYYTDSVKGRFTISRDNAKNMVYLQMNSLKPEDTAVYYCAAGSRRLHYS | |
| | | SDINYWGQGTQVTVSS | |
| 36 | NBX0340 | QVQLQESGGGLVQAGESLRLSCLAAGRTFSTSTLGWFRQAPGLEREFVAAIRY | Cpa |
| | | TSDYTARTTDYADSVKGRFAISRDYIKQAVYLQMNNLKPEDTAVYYCAAAKYG | |
| | | MGYSDPSGYTYWGQGTQVTVSS | |
| 37 | NBX0341 | QVQLQESGGGLVQAGGSLRLSCAASSRTFSNYAMAWFRQTPGKEREFLAAIT | Cpa |
| | | GDTAFTIYADSVKGRFTISRDNPKNTLYLQMNSLKAEDTAVYYCAARQWNPT | |
| | | MRERDYGYWGQGTEVTVSS | |
| 38 | NBX0342 | QVQLQESGGGLVQAGGSLRLSCAASGRRFRLYHMGWFRQAPGKEREFVAVI | Cpb2 |
| | | SWSGGTTVYADSVKGRFTISRDNEKNAGYLQMNSLKPEDTAVYYCAVDRLIE | |
| | | SFSDPTAWPRMDYWGKGALVTVSS | |
| 39 | NBX0343 | QVQLQESGGGLVQPGGSLRLSCAASGFTFSNSAMSWMRQAPGKGVEWVS | Cpb2 |
| | | SINIGGGTTSYADSVAGRFTISRDNAKNTLYLQMNSLKPEDTAVYYCAKGLAST | |
| | | IRGQGTQVTVSS | |
| 40 | NBX0344 | QVQLQESGGGLVQPGGSLRLSCAASGFTFSNSAMSWMRQAPGKGVEWVS | Cpb2 |
| | | SINIGGGTRRYAESVAGRFTISRDNAKNTLYLQMNSLKPEDTAVYYCAKGLAST | |
| | | IRGQGTQVTVSS | |
| 41 | NBX0345 | QVQLQESGGGLVQAGGSLRLSCAASGRKFRLYHMGWFRQAPGKEREFVAVI | Cpb2 |
| | | SWSGGSTVYADSVKGRFTISRDNEKNAGYLQMNSLKPEDTAVYYCAVDRLIES | |
| | | FSDPTAWPRMDYWGKGALVTVSS | |
| 42 | NBX0346 | QVQLQQSGGGLVQPGGSLRLSCAASGFTFSNSAMSWMRQAPGKGVEWVS | Cpb2 |
| | | SINIGGGTRYADSVAGRFTIYRDNAKNTLYLQMNSLKSEDTAVYYCAKGLASTI | |
| | | RGQGTQVTVSS | |
| 43 | NBX0347 | QVQLQESGGGSVQAGGSLRLSCAASGRTFSSYDMGWFRQAPGKEREWVAS | Cpb2 |
| | | ISYNIYYADFVKGRFTISKDNAKNTVSLQMNSLKPEDTAVYYCAAVQRRGSYSY | |
| | | DRAQSYDYWGQGTQVTVSS | |
| 44 | NBX0348 | QVQLQESGGGLVQPGGSLRLSCAASGFTFSNSAMSWMRQAPGKGVEWVS | Cpb2 |
| | | SIEIGGTRRYAESVAGRFTISRDNAKNTLYLQMNSLKAEDTAVYYCAKGLASTI | |
| | | RGQGTQVTVSS | |
| 45 | NBX0349 | QVQLQESGGGLVQPGGSLRLSCAASGFTFSNSPMSWMRQAPGKGVEWVSS | Cpb2 |
| | | INIGAGTTRYAESVAGRFTIARDNAKNTLYLQMNSLKPEDTAVYYCAKGLASTI | |
| | | RGQGTQVIVSS | |
| 46 | NBX0350 | QVQLQESGGGLVQPGGSLRLSCAASGFTFSNSAMSWMRQAPGKGVEWVS | Cpb2 |
| | | SINIGGGDKRYAESVAGRFTISRDNAKNTLYLQMNSLKFEDTAVYYCAKGLAST | |
| | | IRGQGTQVTVSS | |
| 47 | NBX0351 | QVQLQESGGGLVQPGGSLRLSCAASGFTFSNSAMSWMRQAPGKGVEWVS | Cpb2 |
| | | SIETGGTKRYAESVAGRFTISRDNAKNTLNLQMNSLKPEDTAVYYCAKGLASTI | |
| | | RGQGTQVTVSS | |
| 48 | NBX0352 | QVQLQQSGGGLVQPGGSLRLSCAASGFTFSNSPMSWMRQAPGKGVEWVS | Cpb2 |
| | | SINIGEGTTRYAESVAGRFTISRDNVKNTLYLQMNSLKPEDTAVYYCAKGLASTI | |
| | | RGQGTQVTVSS | |
| 49 | NBX0353 | QVQLQESGGGLVQPGGSLRLSCAASGFTFSNSPMSWMRQAPGKGVEWVSS | Cpb2 |
| | | INIGGDTRRYAESVAGRFTISRDNAKNTLYLQMNSLKSEDTAVYYCAKGLASTI | |
| | | RGQGTQVTVSS | |
| 50 | NBX0354 | QVQLQESGGGLVQPGGSLRLSCAASGFTFSNSAMSWMRQAPGNGVEWVS | Cpb2 |
| | | SVNIDGGRRYAEAVAGRFTISRDNAKNTLYLQMNSLKPEDTAVYYCAKGLAST | |
| | | IRGQGTQVTVSS | |
| 51 | NBX0355 | QVQLQESGGGLVQPGGSLRLSCAASGFTFSNSAMAWMRQAPGKGVEWVS | Cpb2 |
| | | SISIDGGRRYAEAVAGRFTISRDNAKNTLYLQMNSLKPEDTAVYYCAKGLASTI | |
| | | RGQGTQVTVSS | |
| 52 | NBX0356 | QVQLQESGGGLVQAGGSLRLSCAASGGKFTLYHMGWFRQTPGKEREFVAVI | Cpb2 |
| | | SWSGRSTVYADSVKGRFTISRDNDKNAGYLQMNSLKPEDTAIYYCAVDRLIEK | |
| | | FSDPTAWPRMDSWGRGTLVTVSS | |
| 53 | NBX0357 | QVQLQESGGGLVQAGDSLRLSCAASGRTASMGWFRQAPGTQREFVATITRS | Cpb2 |
| | | SIYTDYSDSVKGRFAISRDNAKNTVYLQMNSLKPEDTAVYYCAADSTMSGSSR | |
| | | YSSDYAYWGQGTQVTVSS | |
| 54 | NBX0358 | QVQLQESGGGLVQPGGSLRLSCAASGFTFSNSPMSWMRQAPGKGVEWVSS | Cpb2 |
| | | IDIGGNRRYAEAVAGRFTISRDNAKNTLYLQMNSLKPEDTAVYYCAKGLASTIR | |
| | | GQGTQVTVSS | |
| 55 | NBX0359 | QVQLQESGGGLVQAGGSLRLSCAVSGRRFTLYHMGWFRQRPGKEREFVAVI | Cpb2 |
| | | SWSGGSTVYADSVKGRFTISRDNEKNAGYLQMNSLKPEDTAVYYCAVDRLIES | |
| | | FSDPTAWPRMDYWGKGALVTVSS | |
| 56 | NBX0360 | QVQLQQSGGGLVQAGGSLRLSCAASGRRFSLYHMGWFRQAPGKEREFVAVI | Cpb2 |
| | | SWSGGTTVYADSVKGRFTISRDNEKNAGYLQMNSLKPEDTAVYYCAVDRLIE | |
| | | SFSDPTAWPRMDYWGKGALVTVSS | |
| 212 | NBX0361 | QVQLQESGGGLVQAGGSLRLSCAASGSIFSIRVMGWYRQAPGKQRELVATM | NetB |
| | | SRGGTINYADSVRGRFTISRDNAKSTVYLQMNSLKPEDTAVYYCAALLDSYYW | |
| | | GQGTQVTVSS | |
| 213 | NBX0362 | QVQLQESGGGLVQAGGSLRLSCVVSGSIMSIRVMGWYRQAPGKQRELVAT | NetB |
| | | MSRGNTINYADSVRGRFTISRDNAKSTVFLEMNSLKPEDTAVYYCAALLDSYY | |
| | | WGQGTQVTVSS | |
| 214 | NBX0363 | QVQLQESGGGLVQAGGSLRLSCAASASIISIRVMGWYRQAPGKQRELVATM | NetB |
| | | SRGGTINYADSVRGRFTISRDNAKSTVYLQMNSLKPEDTDVYYCAALLDSYYW | |
| | | GQGTQVTVSS | |
| 215 | NBX0364 | QVQLQESGGGLVQAGGSLRLSCVVSGSIMSIRVMGWYRQAPGKQRELVAT | NetB |
| | | MSRGGTINYADSVRGRFTISRDNAKSTVYLQMNSLKPEDTAVYYCTALLDSYY | |
| | | WGQGTQVTVSS | |
| 216 | NBX0365 | QVQLQESGGGLVQAGGSLRLSCAASGSIFSIRVMGWYRQAPGKQRELVATM | NetB |
| | | SRGGTINYADSVRGRFTISRDNAKSTVYLQMNSLKPEDTAVYYCTALLDSYYW | |
| | | GQGTQVTVSS | |
| 217 | NBX0366 | QVQLQESGGGLVQAGGSLRLSCVVSGSIMSIRVMGWYRQAPGKQRELVAT | NetB |
| | | MSRGGTINYADSVRGRFTISRDNAKNTVYLQMTSLKPEDTAVYYCTALLDSYY | |
| | | WGQGTQVTVSS | |
| 218 | NBX0367 | QVQLQESGGGLVQAGGSLRLSCAASGSIFSIRVMGWYRQAPGKQRELVATM | NetB |
| | | SRGGTINYADSVRGRFTISRDNAKSTVYLQMNSLKPEDTDVYYCAALLDSYYW | |
| | | GQGTQVTVSS | |
| 219 | NBX0368 | QVQLQESGGGLVQAGGSLRLSCVVSGSIMSIRVMGWYRQAPGKQRELVAT | NetB |
| | | MSRGNTINYADSVRGRFTISRDNAKNTVYLQMTSLKPEDTAVYYCAALLDSYY | |
| | | WGQGTQVTVSS | |
| 220 | NBX0369 | QVQLQESGGGLVQAGGSLRLSCAASASIFSIRVMGWYRQAPGKQRELVATM | NetB |
| | | SRGNTINYADSVRGRFTISRDNAKSTVYLQMTSLKPEDTAVYYCAALLDSYYW | |
| | | GQGTQVTVSS | |
| 221 | NBX0370 | QVQLQESGGGLVQAGGSLRLSCVVSGSIMSIRVMGWYRQAPGKQRELVAT | NetB |
| | | MSRGGTINYADSVRGRFTISRDNAKSTVYLQMNSLKPEDTAVYYCAALLDSYY | |
| | | WGQGTQVTVSS | |
| 222 | NBX0371 | QVQLQESGGGLVQAGGSLRLSCAASASIISIRVMGWYRQAPGKQRELVATM | NetB |
| | | SRGGTINYADSVRGRFTISRDNAKSTVYLQMNSLKPEDTAVYYCTALLDSYYW | |
| | | GQGTQVTVSS | |
| 223 | NBX0372 | QVQLQESGGGLVQAGGSLRLSCAASASIISIRVMGWYRQAPGKQRELVATM | NetB |
| | | SRGGTINYADSVRGRFTISRDNAKSTVYLQMNSLKPEDTAVYYCAALLDSYYW | |
| | | GQGTQVTVSS | |
| 224 | NBX0373 | QVQLQESGGGLVQAGGSLRLSCAASASIMSIRVMGWYRQAPGKQRELVAT | NetB |
| | | MSRGNTINYADSVRGRFTISRDNAKSTVFLEMNSLKPEDTAVYYCAALLDSYY | |
| | | WGQGTQVTVSS | |
| 225 | NBX0374 | QVQLQESGGGLVQAGGSLRLSCAASASIMSIRVMGWYRQAPGKQRELVAT | NetB |
| | | MSRGGTINYADSVRGRFTISRDNAKSTVYLQMNSLKPEDTDVYYCAALLDSYY | |
| | | WGQGTQVTVSS | |
| 226 | NBX0375 | QVQLQESGGGLVQAGGSLRLSCVVSGSIMSIRVMGWYRQAPGKQRELVAT | NetB |
| | | MSRGGTINYADSVRGRFTISRDNAKSTVYLQMNSLKPEDTDVYYCAALLDSYY | |
| | | WGQGTQVTVSS | |
| 227 | NBX0376 | QVQLQESGGGLVQAGGSLRLSCAASASIISIRVMGWYRQAPGKQRELVATM | NetB |
| | | SRGGTINYADSVRGRFTISRDNAKSTVYLQMTSLKPEDTAVYYCAALLDSYYW | |
| | | GQGTQVTVSS | |
| 228 | NBX0377 | QVQLQESGGGLVQAGGSLRLSCVVSGSIMSIRVMGWYRQAPGKQRELVAT | NetB |
| | | MSRGNTINYADSVKGRFTISRDNAKSTVFLQMNSLKPEDTDVYYCAALLDSYY | |
| | | WGQGTQVTVSS | |
| 229 | NBX0378 | QVQLQESGGGLVQAGGSLRLSCAASASIMSIRVMGWYRQAPGKQRELVAT | NetB |
| | | MSRGNTINYADSVRGRFTISRDNAKSTVYLQMNSLKPEDTAVYYCAALLDSYY | |
| | | WGQGTQVTVSS | |
| 230 | NBX0379 | QVQLQESGGGLVQAGGSLRLSCVVSGSIMSIRVMGWYRQAPGKQRELVAT | NetB |
| | | MSRGNTINYADSVRGRFTISRDNAKSTVYLQMNSLKPEDTAVYYCAALLDSYY | |
| | | WGQGTQVTVSS | |
| 231 | NBX0380 | QVQLQESGGGLVQAGGSLRLSCVVSGSIISIRVMGWYRQAPGKQRELVATM | NetB |
| | | SRGGTINYADSVRGRFTISRDNAKSTVFLEMNSLKPEDTAVYYCAALLDSYYW | |
| | | GQGTQVTVSS | |
| 232 | NBX0381 | QVQLQESGGGLVQAGGSLRLSCAASASIISIRVMGWYRQAPGKQRELVATM | NetB |
| | | SRGNTINYADSVRGRFTISRDDAKNTVYLQMNSLRPDDTAVYYCAALLDSYY | |
| | | WGQGTQVTVSS | |
| 233 | NBX0501 | QVQLQESGGGLVQAGGSLRLSCAASGSIFSINVMGWYRQAPGKQRDLVALIT | NetB |
| | | SGGSTTYADSVKGRFTISRDNAKNTVYLQMNSLKPEDTAVYYCNAAQSRTSW | |
| | | LFPDEYDYWGQGTQVTVSS | |
| 234 | NBX0502 | QVQLQESGGGLVQAGGSLRLSCAASGRTFSIYAMGWFRQAPGKEREFVAVI | NetB |
| | | NRGGGTTTYADSVKGRFTISRDNTKNTVSLQMNSLKPDDTAVYYCAADRVTD | |
| | | TYYYLNPESYDYWGQGTQVTVSS | |
| 235 | NBX0503 | QVQLQESGGGLVQAGGSLRLSCAASGSGRRVGYMAWYRQTPGKQRESVAT | NetB |
| | | ISRAGATKYADSVKDRFTISRDNAKDTVYLQMNSLKPDDTAVYYCFASLIDAG | |
| | | TYWGQGTQVTVSS | |
| 236 | NBX0504 | QVQLQESGGGLVQAGGSLRLSCAASGRTFSIYAMGWFRQAPGKEREFVAVI | NetB |
| | | NRSGGTTTYADSVKGRFTISRDNTKNTVSLQMNSLKPDDTAVYYCAADRVTD | |
| | | TYYYLNPESYDYWGQGTQVTVSS | |
| 237 | NBX0505 | QVQLQESGGGLVQAGGSLRLSCAASGMSFSLGTIYWYRQAPGKQREFVAFIT | NetB |
| | | NADTTMYANSVKGRFTISRDNGKNTVFLLMNNLKPEDSAVYYCNTATSWGQ | |
| | | GTQVTVSS | |
| 238 | NBX0506 | QVQLQESGGGLVQAGGSLRVSCAASGSGRRVGYMAWYRQTPGKQRELVAT | NetB |
| | | ISRAGATNYADSVKDRFTISRDNAKNTVYLQMNSLKPDDTAVYYCFASVFDA | |
| | | GTYWGQGTQVTVSS | |
| 239 | NBX0507 | QVQLQESGGGLVQAGGSLRLSCAASGSGRRVGYMAWYRQTPGKQRELVATI | NetB |
| | | SRAGATNYADSVKDRFTISRDNAKNTVYLQMNSLKPDDTAVYYCFASIFDAGT | |
| | | YWGQGTQVTVSS | |
| 240 | NBX0508 | QVQLQESGGGLVQAGGSLRLSCVASGSGSRINYMAWHRQTPGRQRELVAVI | NetB |
| | | NRTGAANYARSVKDRFTISRDNAKNTVYLQMNDLKPDDTAIYYCFASYLGAG | |
| | | AYWGQGTQVTVSS | |
| 241 | NBX0509 | QVQLQESGGGLVQAGGSLRLSCAASGRTFSTYTVGWFRQAPGKEREFVASIT | NetB |
| | | WNGGTILYADSVKGRFTISRDNAKNTVLLQMNSLKPEDTAVYYCVMGAAGQ | |
| | | GWRYWGQGTQVTVSS | |
| 242 | NBX0510 | QVQLQESGGGLVQAGGSLRLSCVASGSGSRINYMAWHRQTPGRQRELVAVI | NetB |
| | | NRTGAAKYADSVKDRFTVSRDNAENTVYLQMNDLKPDDTAVYYCWASYLGA | |
| | | GTYWGQGIQVTVSS | |
| 243 | NBX0511 | QVQLQESGGGLVQPGGSLRLSCAASGFTFSRNYMSWVRQAPGKGLEWVGSI | NetB |
| | | YSDDSTNYAPSVKGRFTISRDNAANTLYLQMNSLKSEDTAVYYCSKEGGLRGQ | |
| | | GTQVTVSS | |
| 244 | NBX0512 | QVQLQQSGGGLVQAGGSLRLSCAASGSGRRVGYMAWYRQTPGKQRELVAT | NetB |
| | | ISRAGATNYADSVKDRFTISRDNAKNTVYLQMNSLKPDDTAVYYCFASVFDA | |
| | | GTYWGQGTQVTVSS | |
| 245 | NBX0513 | QVQLQESGGGLVQAGGSLRLSCAASGSGRRVGYMAWYRQTPGKQRELVATI | NetB |
| | | SRAGATNYADSVKDRFTISRDNAKNTVYLQMNSLKPDDTAVYYCFASLFDAG | |
| | | TYWGQGTQVTVSS | |
| 246 | NBX0514 | QVQLQESGGGLVQAGGSLRLSCAASGRTFSGRTMAWFRQAPGKEREFVAAI | CnaA |
| | | TWSGGTTYYPDSVKGRFTISRDIPKNTLYLQMNSLKSEDTAVYYCASDGPWR | |
| | | ATTPDAYDYWGQGTQVTVSS | |
| 247 | NBX0515 | QVQLQESGGGLVQAGGSLRLSCAASGSIGTIDSMGWYREAPGKRRELVAFIM | CnaA |
| | | FSGRTIYQDSVKGRFSISGDNAKKTVSLQMTSLKPEDTGVYYCYSNQYWGQG | |
| | | TQVTVSS | |
| 248 | NBX0517 | QVQLQQSGGGLVQPGGSLRLSCAASEFSLLFGTIGWFRQAPGKEREGVSCVS | CnaA |
| | | SSDGSTYYADSVKGRFTISRDKAKNTWYLQMHSLKPEDTAVYYCATRCTVVP | |
| | | GITWGQGTQVTVSS | |
| 249 | NBX0518 | QVQLQESGGGVVQAGGSLRLSCVAPGSITRVGGMGWYRQPPGKERELVALI | CnaA |
| | | NEVGNTNYGDSVKGRFTISRDNAKKTVYLEMNSLKPEDTAVYYCWIPPIPWG | |
| | | QGTQVTVSS | |
| 250 | NBX0519 | QVQLQESGGGLVQPGGSLRLSCATSPFSLRLGVVGWFRQAPGREREGVSCIS | CnaA |
| | | SSEGSTHYADSVKGRFTISRDNAKNTVYLQMNSLKPEDTAVYYCATRCTVVPG | |
| | | ITWGQGTQVTVSS | |
| 251 | NBX0520 | QVQLQESGGGLVQAGDSLRLSCAASARTSSSRAMGWFRQTPVREREFVAAIS | CnaA |
| | | WSGGRTAYADSVKGRFTLSKYDKDTVSLTMNSLKPEDTAVYYCAARRSDFTG | |
| | | DYAYSGRSAYDYWGQGTQVTVSS | |
| 252 | NBX0521 | QVQLQESGGGSVQAGGSLRLSCAASGSTFIFDKMDWYRQTPEKSRELVATL | CnaA |
| | | MSRGDPYYLDSVKGRFTITRDNAKNTVYLQMNSLKPEDTAVYVCRGRAGERV | |
| | | YWGQGTQVTVSS | |
| 253 | NBX0522 | QVQLQESGGGLVQPGGSLRLSCAASGRTFSGVIVGWFRQAPGKEREFLATTL | CnaA |
| | | WSGGSTYYTDSVKGRFTISRDVAKNMVYLQMNSLKPEDAAIYYCAAKYGGSL | |
| | | SYMHPTGYTYWGQGTQVTVSS | |
| 254 | NBX0523 | QVQLQESGGGLVQAGGSLRLSCAASRIVFTISTMAWFRQAPGKEREFVASIN | CnaA |
| | | RSGALTSHANSVKGRFTISRDAAKNTVYLQMNSLKDEDTAIYYCAASKANMP | |
| | | ALPANYDYWGQGTQVTVSS | |
| 255 | NBX0524 | QVQLQESGGGVVQAGGSLRLSCVAPGSITRLGSMGWYRQPPGKQRELVALI | CnaA |
| | | TAVGNTNYGDSVKGRFTISRDNAKKMVYLEMNSLKPEDTAVYYCWIPPIPW | |
| | | GQGTQVTVSS | |
| 256 | NBX0525 | QVQLQESGGGVVQAGGSLRLSCVAPGSITRLGGMGWYRQTPGKQRELVALI | CnaA |
| | | DTVGNTNYGESVKGRFTISRDNAKKMVYLEMNSLKPEDTAVYYCWIPPIPWG | |
| | | QGTQVTVSS | |
| 257 | NBX0526 | QVQLQESGGGLVQAGDSLTLSCVASERAFMYNMAWFRQAPGKERDFVAVR | CnaA |
| | | NWNVERTNYADFAKGRFTISRDAAKKVMYLKMNNLKPEDTAVYYCATTRV | |
| | | WPTQHQMGQIEYWGQGTQVTVSS | |
| 258 | NBX0527 | QVQLQESGGGLVQAGGSLRLSCAASSSFNTMGWYRQAPGKQRELVAGITSG | CnaA |
| | | GTIKYGDSVKGRFTISGDNAKNTVYLQMDSLKPEDTAVYYCVADWQYGSTW | |
| | | NYWGQGTQVTVSS | |
| 259 | NBX0528 | QVQLQESGGGLVQAGDSLRLSCAASGRNFDYYSMGWFRQAPGNERIFVAAI | CnaA |
| | | NWRGAVIDYPDSVKGRFTISRDNAKNRVYLQMNSLKPEDTAVYYCAAASSSS | |
| | | RLLEPIGYNYWGQGTQVTVSS | |
| 260 | NBX0529 | QVQLQESGGGLVQAGGSLRLSCAASGSMFSINDMTWYRQAPGKQREMVA | CnaA |
| | | TISSGGTTDYTESVKGRFFVIRDNAKITVYLQMNKLRPEDSGVYYCAGNLKRSE | |
| | | TSYYWKTGQGIQVTVSS | |
| 261 | NBX0530 | QVQLQESGGGLVQTGGSLKLSCATSGRTFSRYHMGWFRQAPGKEREFVAAI | CnaA |
| | | SLSGGGTAFANFVEGRFTISRDNAKNTLYLQMNSLKPEDTAVYYCTADRHEW | |
| | | GRLMKGDYWGQGTQVTVSS | |
| 262 | NBX0531 | QVQLQESGGGSVQAGGSLTVSCSASGRTSNSYNMAWFRQGPGKERELVAAI | CnaA |
| | | SWTGGFTSYTNSVKDRFTISRENAKNTVYLQMNSLKPEDTAVYYCAATSRSLT | |
| | | SAMTREIRAYDYWGQGTQVTVSS | |
| 263 | NBX0532 | QVQLQESGGGLVQAGGSLRLSCAASGSTFSFNKMDWYRQAPEKQRELVATF | CnaA |
| | | MNDGNTYYVDSVKGRFTISRDNAKNTVYLQMNSLKFEDTAVYYCRGRAGME | |
| | | VYWGQGTQVTVSS | |
| 264 | NBX0533 | QVQLQESGGGLVQPGGSLTLSCATSPLTLRLGPIGWFRQAPGKEREGVSCISS | CnaA |
| | | RDDKNYAESVKGRFTISRDNAKNMVYLQMNSLKPEDTAVYYCATRCTVVPGI | |
| | | SWGQGTQVTVSS | |
| 265 | NBX0534 | QVQLQESGGGLVQAGDSLRLSCAASGRNFGYYTMGWFRQAPGNERIFVAAI | CnaA |
| | | TWRGVIHHADSVKGRFTISRDNAKNTVYLQMNSLKPEDTAVYYCAAASSSSR | |
| | | PLEPIGYNYWGQGTQVTVSS | |
| 266 | NBX0535 | QVQLQESGGGLVQAGGSLRLSCTASGDIFSAAGMAWFRQTPGKERDLVAYV | CnaA |
| | | TWDGGTTRYKDSVKGRFTISRDNAKNTVLLQMNSLKPEDTAVYYCAAGNTG | |
| | | PFNLLHSSAQYAYWGQGTQVTVSS | |
| 267 | NBX0536 | QVQLQESGGGLVQAGGSLRLSCATSPLTLRLGAIGWFRQAPGKEREMVSCIT | CnaA |
| | | STEDKNYADSVKGRFTISRDNAKTMVYLQMNSLKLEDTAVYYCATRCTVVPGI | |
| | | SWGQGTQVTVSS | |
| 268 | NBX0537 | QVQLQESGGGVVQAGGSLRLSCVAPGSITRIGGMGWYRQPPGKQRELVALI | CnaA |
| | | NTVGNTNYGDSVKGRFTISRDNAKKTVYLEMNSLKPEDTAVYYCWIPPLPWG | |
| | | QGTQVTVSS | |
| 269 | NBX0538 | QVQLQQSGGGLVQAGGSLRLSCTASGRSFSRYIMGWFRQAPGKERESVARI | NetB |
| | | APSGGSAYYADSVKGRFTISRDNAKNIVYLQMNNLKSEDTAVYHCAARYDM | |
| | | DYEYKTWGPGTQVTVSS | |
| 270 | NBX0539 | QVQLQESGGGLVQAGGSLRLSCVASGSGSRIGFMAWHRQTPGRQRELVAVI | NetB |
| | | NRTGATRYADSVKDRFTISRDNAKNTVYLQMNDLKPDDTALYYCFASVVDAG | |
| | | TYWGQGTQVTVSS | |
| 271 | NBX0540 | QVQLQESGGGLVQPGGSLRVSCAASGLTFSDYAMGWFRQAPGQEREFVARI | NetB |
| | | SLTAASTLYADSVRGRFTISRDNAKNTVYLQMNSLRPDDTAVYYCAAQGRILR | |
| | | GRGLFKASDYDYWGQGTQVTVSS | |
| 272 | NBX0541 | QVQLQQSGGGSVQTGGSLALSCAASGTISIFDPMGWYRQAPGKQRELVASIS | NetB |
| | | EGSTNYANSVKGRFTISRDNAKKTVSLQMNSLEPADTAVYYCRLSRYYNSNIY | |
| | | WGQGTQVTVSS | |
| 273 | NBX0542 | QVQLQESGGGLVQAGGSLRLSCAASRNIYGINVIAWYRQAPGKQREMVARS | NetB |
| | | ANGGTTRYADSVKGRFTISRDNVKNIVYLQMSSLKPEDTAAYYCKAELYTLQH | |
| | | NYEYWGQGTQVTVSS | |
| 274 | NBX0543 | QVQLQESGGGSVQTGGSLALSCVASGTLSLFDPMGWYRQAPGKQRELVASI | NetB |
| | | SGLSTNYANSVKGRFTISRDDAKKTVSLQMNSLEPADTAVYYCHLSRYYNSNIY | |
| | | WGQGTQVTVSS | |
| 275 | NBX0544 | QVQLQESGGGLVQAGGSLRLSCAASGRVLSINAMGWYRQAPGKRREMVAR | NetB |
| | | ITNGGSTNYAGSVKGRFTISRENTKNTMYLQMNSLKPEDTAVYYCLAEERPYY | |
| | | GGPLEYWGQGTQVTVSQ | |
| 276 | NBX0545 | QVQLQESGGGLVQAGGSLRLSCAASRTTFRVGTMAWFRQDPGKQRELVAGI | NetB |
| | | TSGGSTNYADSVKGRFTISRDNAKNTIYLQMNSLKPEDTGIYVCFANIVDRPVS | |
| | | WGQGTQVTVSS | |
| 277 | NBX0546 | QVQLQQSGGGAVQAGGSLTLSCVASGSGSRIGLMAWYRQTPGRQRELVAVI | NetB |
| | | KGTGTTRYADSVKDRFTISRDNAKNTMYLQMNDLKPDDTALYYCFASVLGAG | |
| | | TYWGQGTQVTVSS | |
| 278 | NBX0547 | QVQLQESGGGSVQTGGSLALSCAASGTISLFDSMGWYRQAPGKQRELVASIT | NetB |
| | | EGSTNYANSVKGRFTISRDNAKKTVSLQMNSLEPADTAVYYCRLSRYYNSNIY | |
| | | WGQGTQVTVSS | |
| 279 | NBX0548 | QVQLQQSGGGLVQSGGSLRLSCAASETSLNFDDMRWYRQTPGKRREWVAII | NetB |
| | | NTFPAGTTASYADSVKGRFTISKVNGENTVHLQMNRLKPEDTAVYYCNAGDY | |
| | | WGQGTQVTVSS | |
| 280 | NBX0549 | QVQLQESGGGLVQAGGSLRLSCTASGSDSSINYMGWYRQAPGKQRVLLAAI | NetB |
| | | SRDGRSNYADSVRGRFTISRDNAKNTVDLQMNSLKPEDTAVYYCYVDPLGRV | |
| | | PRWGQGTQVTVSS | |
| 281 | NBX0550 | QVQLQESGGGAVQAGGSLTLSCVASGTVNLMAWYRQTPGRQRELVAVIKG | NetB |
| | | TGTTRYADSVKDRFTISRDNAKNTMYLQMNDLKPDDTALYYCFASVLGAGTY | |
| | | WGQGTQVTVSS | |
| 282 | NBX0551 | QVQLQESGGGLVQAGGSLRLSCAASGSIFSRNIILWHRQAPGKQRELVGGINT | NetB |
| | | GGRTNYESSVKGRFTISRDNAKNTVYLQMDRLKPEDTAVYYCNAPSLGYWG | |
| | | QGTQVTVSS | |
| 283 | NBX0552 | QVQLQQSGGGLVQAGGSLRLSCVASGSGSINYMAWHRQTPGRQRELVAVI | NetB |
| | | NRTGAARYADSVKDRFTISRDNAENTMYLQMNDLKPDDTAVYYCFASALGA | |
| | | GVYWGQGTQVTVSS | |
| 284 | NBX0553 | QVQLQESGGGLVQPGGSLRLSCAASGSGWRVGYMAWYRQTPGKQRELVA | NetB |
| | | TISRAGATRYEDSVKDRFTISRDNAKNTVYLQMNSLKPDDTAVYYCFASIIDAG | |
| | | TYWGQGTPVTVSS | |
| 285 | NBX0561 | QVQLQESGGGLVQAGGSLRLSCTASGENFSTYVMGWFRQAPGKEREFVAA | CnaA |
| | | HNWRGGGTYYADSVKGRFTISRDHAKNTVYLEMNSLKPEDTAVYYCAARSG | |
| | | GSYTYTGSYHYWGQGTQVTVSS | |
| 286 | NBX0801 | QVQLQESGGGLVQAGDSLRLSCAAAGRTFSSYAMGWFRQAPGKEREFVATI | CnaA |
| | | SRSGGSTYYADSVKGRFTISRDNAKNTVYLQMNSLKPEDTAVYYCAANRYGSS | |
| | | SYQGQYASWGQGTQVTVSS | |
| 287 | NBX0802 | QVQLQESGGGLVQAGGSLRLSCAASGRTFSSYHMGWFRQAPGKEREFVATI | CnaA |
| | | SRSGGFTSYADSVKGRFTISRDNAKNTVWLQMNSLKPEDTAVYYCAAQQWP | |
| | | DPRNPNGYDYWGQGTQVTVSS | |
| 288 | NBX0803 | QVQLQESGGGLVQAGGSLRLACAASGRTFINYGMAWFRQSPGKEREFVAAV | CnaA |
| | | SISGAGTAYVEPVKDRFTISRDNTKNTLYLQMNTLKPEDTALYYCAAAKAGH | |
| | | WGRDANYDYWGQGTQVTVSS | |
| 289 | NBX0804 | QVQLQQSGGGLVQAGGSLRLSCSASGRTLTAYGMAWFRQSPGKEREFVAA | CnaA |
| | | VSLSGASTAYVEPVKDRFTISRDNTQNTVYLQMNSLKPEDTALYYCAAAKAG | |
| | | QWGRDAKYDYWGQGTQVTVSS | |
| 290 | NBX0805 | QVQLQQSGGGLVQAGGSLRLSCAASGRTFSTYAMGWFRQAPGKEREFVAGI | CnaA |
| | | SWSGGRISYTDSVKGRFTISRDNAKNTVYLQMNSLKPEDTAVYYCTADLKGL | |
| | | WALGLPGHYASWDSWGQGTQVTVSS | |
| 291 | NBX0806 | QVQLQESGGGLVQPGGSLRLSCAASGSIGSINIMDWYRQAPGKQRDLVATFT | CnaA |
| | | SGGSTVYADSVKGRFTISRDNAKDTVYLQMNSLKPEDTAVYYCRARRGWAIY | |
| | | WGQGTQVTVSS | |
| 292 | NBX0807 | QVQLQQSGGGLVQAGDSLRLSCAASGRTFSSYGMGWFRQATGKEREFVAGI | CnaA |
| | | SRTGSGTAYADSVKSRFTISRDNAKNTVYLQMNSLKAEDTAVYYCAADSGGS | |
| | | WGRGTTYDYWGQGTQVTVSS | |
| 293 | NBX0808 | QVQLQQSGGGSVQAGGSLRLSCRASARASSIGAMAWFRQAPGKDRELVAA | CnaA |
| | | VTAGADTTYYRDFVKGRFTLSRDNAKNTVYLQMNSLKLDDTAVYYCAAYNTA | |
| | | GWGEPHQSYRYWGQGTQVTVSS | |
| 294 | NBX0809 | QVQLQESGGGLVQAGGSLKLSCVASGLTFGNYDMAWFRQAPGKEREFVTHI | CnaA |
| | | SSSGAYTSYAYFVKGRFTISRDIAKNTVYLQMNSLKPEDTAIYYCAGRRSVVVR | |
| | | SFDYDYWGQGTQVTVSS | |
| 295 | NBX0810 | QVQLQQSGGGLVHPGGSLRLSCAASGRIFNANGMYWYRQAPGKQRELVAS | CnaA |
| | | LYRSGSTNYLDSVKGRFTISRDNAKNTVYLQMNSLKPEDTAVYYCNVNWALH | |
| | | DSWGQGTQVTVSS | |
| 296 | NBX0811 | QVQLQESGGGLVQAGDSLRLSCAASERTFSSDGMAWFRQATGKEREFVAGI | CnaA |
| | | SRTGSATAYAEFVKSRFTISRDNAKNTVYLQMNSLKAEDTAVYYCAANSGGH | |
| | | WWRGATYDYWGQGTQVTVSS | |
| 297 | NBX0812 | QVQLQESGGGLVQAGGSLRLSCTASGTIFSANGMYWYRQALGQRRELVASL | CnaA |
| | | YRDGSTNYADSVKGRFTISRDNAKNTVYLQMNSLKPEDTAVYYCNVNWALH | |
| | | DSWGQGTQVTVSS | |
| 298 | NBX0847 | QVQLQESGGGVVQAGDSLRLSCTASTRASIVGAMAWFRQAPGRNRDIVAAI | CnaA |
| | | AAGSPSTPYYADSVKGRFAISRDNAKNTVYLQMNSLKSEDTAIYYCAAYNTAN | |
| | | WGQPHQSYRHWGQGIQVTVSS | |
| 299 | NBX0866 | QVQLQESGGGLVQPGGSLRLSAAASGSILNINVMAWFRQAPGKQREWVASI | CnaA |
| | | YRDGSTYYADSVKGRFTISRDNAKNTVYLQMNSLKPEDTAVYYCNVVTYGSN | |
| | | RRDFWGQGTQVTVST | |
| 300 | NBX0867 | QVQLQESGGGLVQAGDSLRLSCAASGRTFSSYAMGWFRQAPGKDREFVSTI | CnaA |
| | | SRSGGSTYYADSVKGRFTISRDNAKNTVYLQMNSLKPEDTAVYYCAANRYGSS | |
| | | SYQGQYGSWGQGTQVTVSS | |
| 301 | NBX0868 | QVQLQQSGGGLVQAGDSLRLSCAASGRTFSSYAMGWFRQAPGKEREFVASI | CnaA |
| | | SRSGGSTYYADSVKVRFTISRDNAKNTVYLQMNSLKPEDTAVYYCAANRYGSS | |
| | | SYQGQYDYWGQGTQVTVSS | |
| 302 | NBX0869 | QVQLQESGGGLVQAGGSLRLSCTASGTIFSINGMYWYRQALGKRRELVASLY | CnaA |
| | | RGGSTNYADSVKGRFTISRDNAKNTVYLQMNSLKPEDTAVYYCNVNWALQD | |
| | | SWGQGTQVTVSS | |
| 303 | NBX0870 | QVQLQESGGGLVQAGGSLRLSCAASTSDGSINVMDWYRQTPGKQRDLVATI | CnaA |
| | | TSLGSQVYADSVKGRFTISRDNAKDTVYLQMNSLKPEDTAVYYCRARRGWAI | |
| | | YWGQGTQVTVSS | |
| 304 | NBX0871 | QVQLQQSGGGLVQAGGSLRLSCAASGRTFNIYAMGWFRQAPGKEREFVAGI | CnaA |
| | | SDSGGSANYADSVKDRFTISMDNAKNTVYLQMNSLKPEDTAVYYCAADLTGL | |
| | | WALGLPGHYASWDSWGQGTQVTVSS | |
| 305 | NBX0872 | QVQLQESGGGLVQPGGSLRLSCAASGFTFRSSAMSWVRQVPGKGLEWVSSI | CnaA |
| | | GSDGENIYYADAVKGRFTISRDNAKNTMYLQMNSLKLEDTAVYYCQLGRTVL | |
| | | DYFKGQGTQVTVSS | |
| 306 | NBX0873 | QVQLQESGGGLVQPGGSLRLSCAASGRTFINYGMAWFRQSPGKEREFVAAV | CnaA |
| | | SSSGAGTAYVEPVKDRFTISRNNTKNTVYLQMNSLKPEDTALYYCAAAKAGQ | |
| | | WGRYANYDYWGQGTQVTVSS | |
| 307 | NBX0874 | QVQLQQSGGGLVQAGDSLRLSCAASGRTFSSYAMGWFRQAPGKEREFVAAI | CnaA |
| | | SRSGGTTYYADSVKGRFTISRDNAKNTVYLQMNTLKPEDTAVYYCAANPYGSS | |
| | | SYQGQYGSWGQGTQVTVSS | |
| 308 | NBX0875 | QVQLQQSGGGLVQAGDSLRLSCAASGRAFSGYAMGWFRQAPGREREFVAA | CnaA |
| | | ISRGGGTTYYADSVKGRFTISRDNAKNTVYLQMNSLKPEDTAVYYCAANRYGS | |
| | | SSYQGQYGSWGQGTQVTVSS | |
| 309 | NBX0876 | QVQLQESGGGLVQAGGSLRLSCAASGRTFINYGMAWFRQSPGKEREFVAAV | CnaA |
| | | SSSGAGTAYVEPVKDRFTISRDNTKNTVYLQMDTLKPEDTALYYCAAAKAGH | |
| | | WGRDANYDYWGQGTQVTVSS | |
| 310 | NBX0877 | QVQLQESGGGMVEPGGSLRLSCAASGSISSITFMGWHRQAPGKEGEFVALIA | NetB |
| | | RSGTTTYADSVKGRFSISRDNAKNTVYLQMNNLKPEDTALYYCYVDRRGAVP | |
| | | TWGQGTQVTVSS | |
| 311 | NBX0878 | QVQLQQSGGGLVEPGGSLRLSCAASGSISSITFMGWHRQAPGEQGELVALIA | NetB |
| | | RSGTTTYADSVKGRFTISRDNAKNTVYLQMNNLKPEDTALYYCYVDRRDVVP | |
| | | TWGQGTQVTVSS | |
| 312 | NBX0879 | QVQLQESGGGLVQAGGSLRLSCAASGTGFPIITFMGYYRQAPGNQRELVAIIS | NetB |
| | | RGGVAKYGDSVKDRFTISRDNAKNTVYLEMNSLKPDDTAVYYCYADRFSGSP | |
| | | TWGQGTQVTVSS | |
| 313 | NBX0880 | QVQLQESGGGLVQPGGSLRLSCAASVSSIGTMGWFRQAPGKQPELVASISRV | NetB |
| | | GTTNYANSVKGRFTVSRDNAQNTMYLQMNSLKPEDTAVYLCFANVISGPVY | |
| | | WGQGTQVTVSS | |
| 314 | NBX0881 | QVQLQESGGGLVQAGGSLRLSCAASTRFFSNYAMGWFRQAPGKEREFVAAI | NetB |
| | | SRDGAVPLSGNSVPGRFTISRDNAKNTLYLQMNSLKPEDSAVYYCAASRQGN | |
| | | PYAQTSYDYWGQGTQVTVSS | |
| 315 | NBX0883 | QVQLQESGGEVVAPGGSLSLSCVASGSADSIKIMGWYRQAPGKQRELVATIT | NetB |
| | | SGGTTEFAESVKGRFTISRDNAKNTLYMQMNSLSPEDTAVYYCNALVSRRDS | |
| | | AAYFAWGQGTQVTVSS | |
| 316 | NBX0884 | QVQLQESGGGLVQPGGSLRLSCAASESIVSITPMMWYRQAPGKQREWVAIT | NetB |
| | | TRDGAPAYADSVKGRFTISRDSAKNTVYLQMNYLKPEDTAVYFCKARKDSHD | |
| | | YWGQGTQVTVSS | |
| 317 | NBX0885 | QVQLQESGGGLVQAGGSLRLSCAASETIGSIQRMGWYRQAPGKQRELVATR | NetB |
| | | TNGGTTNYGDSVRGRFTISVDVAKNTVYLQMNSLKPEDTAVYYCNAHIREYY | |
| | | STYDYWGQGTQVTVSS | |
| 318 | NBX0886 | QVQLQESGGGLVQPGGSLRLSCSASGSISRIRDMAWHRQVPGKQRELVASIS | NetB |
| | | SGGSTNVADSLKGRFTISRDNGKNTMYLQMDSLKSEDTAVYYCNALFNPIDG | |
| | | PARYYWGQGTQVTVSS | |
| 319 | NBX0887 | QVQLQESGGGLVQPGGSLRLSCSASGSISRIYDMAWHRQVPGKQRELVAGIS | NetB |
| | | RGGSTNVADSLKGRFTISRDNGKNTVYLQMDNLKSEDTAVYYCTALFNPVDG | |
| | | TARYYWGQGTQVTVSS | |
| 320 | NBX0888 | QVQLQESGGGLVQAGGSLRLSCAASGTIFSINVMGWYRQAPGKQRELVASIT | NetB |
| | | SGGQIKYADSVKGRFTTSRDNAKNTVYLQMNSLKPEDTAVYYCNAASSTWPP | |
| | | RDYDYWGQGTQVTVSS | |
| 321 | NBX0889 | QVQLQESGGGLVQPGGSLRLSCAASRSISSIAAMGWYRQAPGKQRELVARIT | NetB |
| | | NGGSTNYADSVKGRFTISRDNAKNTVYLQMNSLKPEDTAVYYCNADERPYYG | |
| | | DSVLSWGQGTQVTVSS | |
| 322 | NBX0890 | QVQLQQSGGGLVQAGGSLRLSCAASGTGFPIITFMGYYRQTPGNQREEVALI | NetB |
| | | NRGGVAKYGDSVKDRFTISRDNAKNTVYLEMNNLKPDDTAVYYCYADRFSGS | |
| | | PTWGQGTQVTVSS | |
| 323 | NBX0891 | QVQLQESGGGLVQAGGSLRLSCAASGRTFSNYHMAWFRQAPGKEREFVAAI | NetB |
| | | SRGTSTTFYRDSVRDRFTISRDNAKNTAYLQMNSLKPEDTAVYYCAADADRST | |
| | | TIYSRDIYDYWGQGTQVTVSS | |
| 324 | NBX0892 | QVQLQESGGGLVQAGDSLRLSCAASEGTFSNYRMGWFRQAPGKEREFVAAI | NetB |
| | | SRDGAVPLSGNSPLGRFTISRDNAKNTLYLQMNSLKPEDTAVYYCAASRQGLP | |
| | | YVETSYDYWGQGTQVTVSS | |
| 325 | NBX0893 | QVQLQESGGGLVQPGGSLRLSCVASGSISSITFMGWYRQVLGEQRELVALSA | NetB |
| | | RRGTTTYADSVKGRFTISRDNAKNTVYLQMNNLKPEDTALYYCYVDRRDEVP | |
| | | TWGQGTQVTVSS | |
| 326 | NBX0894 | QVQLQQSGGGLVQAGGSLRLSCAASGGTFSSYVMAWFRQAPGKEREFLAAI | NetB |
| | | RWSRGSTYYADSVKGRFTVFRDTVENTVYLQMNSLKPEDTAVYYCAADGNP | |
| | | AKLVLDQYGMDYWGKGTLVTVSS | |
| 327 | NBX0895 | QVQLQQSGGGLVEPGGSLRLSCAASGSISEITYMGWHRQAPGEQRELVALIA | NetB |
| | | RVGTTRYADSVKGRFTISRDNAKNTVYLQMNNLKPEDTALYYCYVDQRGVVP | |
| | | TWGQGTQVTVSS | |
| 328 | NBX0896 | QVQLQESGGGSVQAGGSLRLSCRASARASSIGAMAWFRQAPGKDRELVAAV | CnaA |
| | | NAGADTTYYRDFVKGRFTISRDNAKNTVYLQMNSLKLDDTAVYYCAAYNTAG | |
| | | WGEPHQSYRYWGQGIQVTVSS | |
| 329 | NBX0897 | QVQLQESGGGLVQPGGSLSLSCAASGSIFIISTMGWYRQAPGKQRELVATITS | CnaA |
| | | GGSTNYADPVKGRFTISRDNAKNMVYLQMNSLKPEDTAVYYCNAEVHVWG | |
| | | VPGPRDYWGQGTQVTVSS | |
| 330 | NBX0898 | QVQLQESGGGLVQAGDSLRLSCAASGRTFSSYAMGWFRQAPGKEREFVATI | CnaA |
| | | SRSGGSTYYADSVKGRFTISRDNAKNTVYLQMNSLKPEDTAVYYCAANPYGSS | |
| | | SYQGQYASWGQGTQVTVSS | |
| 331 | NBX0899 | QVQLQQSGGGLVQAGGSLRLSCAASGSIFSSNGMYWYRQAPGKQRELVASL | CnaA |
| | | YRSGSTNYADSVKGRFIISRDNAKNTVYLQMNSLKPEDTAVYYCNVNWALHD | |
| | | SWGQGTQVTVSS | |
| 332 | NBX08100 | QVQLQESGGGLVQAGGSLRLSCAASGRTFSAYGMAWFRQSPGKEREFVAAV | CnaA |
| | | SGGGGGTAYAEPVKDRFTISRDNAKNTVYLQMNTLKPEDTALYYCAAATAGH | |
| | | WGRDANYDYWGQGTQVTVSS | |
| 333 | NBX08101 | QVQLQESGGGLVQAGGSLRLSCAASGSIFSSNGMYWYRQAPGKQRELVASL | CnaA |
| | | FRSGSTNYADSVKGRFTISRDNAQNTVYLRMNSLKPEDTAVYYCNVNWALH | |
| | | DSWGPGTQVTVSS | |
| 334 | NBX08102 | QVQLQESGGGLVQAGDSLRLSCAASGRTFSSYAMGWFRQAPGKEREFVAAI | CnaA |
| | | SRSGGTTYYADSVKGRFTISRDNAKNTVYLQMNTLKPEDTAVYYCAANPYGSS | |
| | | SYQGQYGSWGQGTQVTVSS | |
| 335 | NBX08103 | QVQLQESGGGLVQPGGSLRLSCAASGIIHSINVMGWYRQAPGKQRELVAIISS | CnaA |
| | | GGRTTYADSVKGRSTITGDNDKNTVYLQMNSLKPEDTAVYYCTMVWGLRYY | |
| | | WGQGTQVTVSS | |
| 336 | NBX08104 | QVQLQQSGGGFVRPGESLTLSCAASTSIFSSNGMYWYRQAPGKRRELVASLF | CnaA |
| | | RSGSTNYADSVKGRFTISRDNAKNTVYLQMNSLKPEDTAVYYCNVNWALHD | |
| | | SWGQGTQVTVSS | |
| 337 | NBX08105 | QVQLQESGGGLVQAGDSLRLSCAASGRTFSSYAMAWFRQAPGKEREFVAAI | CnaA |
| | | SRGGGTTYYADSVKGRFTISRDNAKNTVYLQMNSLKPEDTAVYYCAANPYGS | |
| | | SSYQGQYGSWGQGTQVTVSS | |
| 338 | NBX08106 | QVQLQESGGGLVQAGGSLRLSCAASGSIFSSNGMYWYRQAPGKQRELVASL | CnaA |
| | | YRSGSTNYADSVKGRFIISRDNAKNTVYLQMNSLKPEDTAVYYCNVNWALHD | |
| | | SWGQGTQVTVSS | |
| 339 | NBX08107 | QVQLQQSGGGEVQPGGSLRLSCAASGSIFSSNGMYWYRQAPGKQRELVASL | CnaA |
| | | YRSGSTNYADSVKGRFIISRDNAKNTVYLQMNSLKPEDTAVYYCNVNWALHD | |
| | | SWGQGTQVTVSS | |
| 340 | NBX08108 | QVQLQESGGGLVQAGGSLRLSCAASRSILSANGMYWYRQAPGKQRELVASL | CnaA |
| | | YRSGSTDYADSVKGRFTISRDDSRDTMYLQMNSLKPEDTAVYYCNVNWALH | |
| | | DSWGQGTQVTVSS | |

TABLE 2
Unique SEQ IDs for V_{H}H CDRs of this disclosure

| | CDR1 | CDR1 | CDR2 | CDR2 | CDR3 | CDR3 | |
|---|---|---|---|---|---|---|---|
| | Amino | SEQ | Amino | SEQ | Amino | SEQ | |
| | Acid | ID | Acid | ID | Acid | ID | |
| NBX | Sequence | NO: | Sequence | NO: | Sequence | NO: | Antigen |
| NBX0301 | QRASSLFAM | 57 | ISWNGDKS | 107 | AAHRASFELGF | 157 | NetB |
| | | | | | ATHDYDF | | |
| NBX0302 | GSIFSISSA | 58 | IFSDGST | 108 | AVDGY | 158 | NetB |
| NBX0303 | GRTLSYWTM | 59 | INWSSGT | 109 | AAHRASFGLGY | 159 | NetB |
| | | | | | QTHEYDF | | |
| NBX0304 | GGTFSSYTM | 60 | ITWNSEVT | 110 | AAGRAGSGFTS | 160 | NetB |
| NBX0305 | GFTLDKYAV | 61 | ISSIDDST | 111 | MTIPLPYGSTCD | 161 | NetB |
| | | | | | IPSRSDLLAINY | | |
| NBX0306 | GFTVPYYYI | 62 | IASSSGKA | 112 | AALRKYGSTCYL | 162 | NetB |
| | | | | | HVLEYDY | | |
| NBX0309 | GSTFNNYMI | 63 | ISGSGAGT | 113 | ARRMSRSGIFGL | 163 | NetB |
| | | | | | RDYDS | | |
| NBX0311 | GRTFSNADM | 64 | ISWSGGRT | 114 | AAGGYSNLPTS | 164 | NetB |
| | | | | | YGY | | |
| NBX0316 | GRAFSTYGM | 65 | ISSSGAGS | 115 | AASTTSWGKFA | 165 | CnaA |
| | | | | | HYIY | | |
| NBX0317 | GGTFSSYIM | 66 | ISWSGGVT | 116 | AADSRISAGGSY | 166 | CnaA |
| | | | | | YEADFGS | | |
| NBX0318 | GSIMSIRVM | 67 | MSRGNTI | 117 | AALLDSYY | 167 | NetB |
| NBX0319 | ASIISIRVM | 68 | MSRGGTI | 118 | TALLDSYY | 168 | NetB |
| NBX0320 | GSIFSIRVM | 69 | MSRGGTI | 119 | AALLDSYY | 169 | NetB |
| NBX0321 | GFTLDKYAV | 70 | ISSIDDST | 120 | MTIPLPYGSTCR | 170 | NetB |
| | | | | | IPSRSDLLAINY | | |
| NBX0322 | GRTFSTYAM | 71 | ITRGGNT | 121 | AADRIIVPRDPMDY | 171 | NetB |
| NBX0326 | GRTFSAIHM | 72 | ISWSGGGT | 122 | AASDTDWGRS | 172 | CnaA |
| | | | | | ASYDY | | |
| NBX0327 | GGTFSSYVM | 73 | ISWSGGVT | 123 | AADSRISAGGSY | 173 | CnaA |
| | | | | | YEADFGS | | |
| NBX0328 | GRTFSSYTM | 74 | ISWSGT | 124 | AVGSRRLYYSSDINY | 174 | Cpa |
| NBX0329 | GLTVSRYTM | 75 | ISWSGT | 125 | AAGSRRLYYSN | 175 | Cpa |
| | | | | | DINY | | |
| NBX0330 | SRTFSNYAM | 76 | INGDTTFT | 126 | AARQWNPTMR | 176 | Cpa |
| | | | | | ERDYGY | | |
| NBX0331 | GRVFENYFM | 77 | TNWNTATNWNT | 127 | AATGSRTYDVV | 177 | Cpb2 |
| | | | | | DYYDY | | |
| NBX0332 | GRTFSSYSM | 78 | ITYSGITT | 128 | AASYSASRSYPF | 178 | Cpb2 |
| | | | | | GEYDY | | |
| NBX0333 | GRTFSSYSM | 79 | ITYSGIST | 129 | AASYSASRSYPF | 179 | Cpb2 |
| | | | | | GEYDY | | |
| NBX0334 | GFTFSNSAM | 80 | INIGGDSR | 130 | AKGLASTI | 180 | Cpb2 |
| NBX0335 | GFTFSNSAM | 81 | IEVGGGR | 131 | SKGLASTI | 181 | Cpb2 |
| NBX0336 | GFTFSNSAM | 82 | IGIDGGR | 132 | AKGLASTI | 182 | Cpb2 |
| NBX0337 | GFTFSNSAM | 83 | IGIGGGTT | 133 | AKGLASTI | 183 | Cpb2 |
| NBX0338 | GRSFSSYTM | 84 | ISWSGT | 134 | AVGSRRLYYSSDINY | 184 | Cpa |
| NBX0339 | GLTVSRYTM | 85 | ISWSGT | 135 | AAGSRRLHYSS | 185 | Cpa |
| | | | | | DINY | | |
| NBX0340 | GRTFSTSTL | 86 | IRYTSDYTAR | 136 | AAAKYGMGYS | 186 | Cpa |
| | | | TT | | DPSGYTY | | |
| NBX0341 | SRTFSNYAM | 87 | ITGDTAFT | 137 | AARQWNPTMR | 187 | Cpa |
| | | | | | ERDYGY | | |
| NBX0342 | GRRFRLYHM | 88 | ISWSGGTT | 138 | AVDRLIESFSDP | 188 | Cpb2 |
| | | | | | TAWPRM | | |
| NBX0343 | GFTFSNSAM | 89 | INIGGGTT | 139 | AKGLASTI | 189 | Cpb2 |
| NBX0344 | GFTFSNSAM | 90 | INIGGGTR | 140 | AKGLASTI | 190 | Cpb2 |
| NBX0345 | GRKFRLYHM | 91 | ISWSGGST | 141 | AVDRLIESFSDP | 191 | Cpb2 |
| | | | | | TAWPRM | | |
| NBX0346 | GFTFSNSAM | 92 | INIGGGT | 142 | AKGLASTI | 192 | Cpb2 |
| NBX0347 | GRTFSSYDM | 93 | ISYNI | 143 | AAVQRRGSYSY | 193 | Cpb2 |
| | | | | | DRAQSYDY | | |
| NBX0348 | GFTFSNSAM | 94 | IEIGGTR | 144 | AKGLASTI | 194 | Cpb2 |
| NBX0349 | GFTFSNSPM | 95 | INIGAGTT | 145 | AKGLASTI | 195 | Cpb2 |
| NBX0350 | GFTFSNSAM | 96 | INIGGGDK | 146 | AKGLASTI | 196 | Cpb2 |
| NBX0351 | GFTFSNSAM | 97 | IETGGTK | 147 | AKGLASTI | 197 | Cpb2 |
| NBX0352 | GFTFSNSPM | 98 | INIGEGTT | 148 | AKGLASTI | 198 | Cpb2 |
| NBX0353 | GFTFSNSPM | 99 | INIGGDTR | 149 | AKGLASTI | 199 | Cpb2 |
| NBX0354 | GFTFSNSAM | 100 | VNIDGGR | 150 | AKGLASTI | 200 | Cpb2 |
| NBX0355 | GFTFSNSAM | 101 | ISIDGGR | 151 | AKGLASTI | 201 | Cpb2 |
| NBX0356 | GGKFTLYHM | 102 | ISWSGRST | 152 | AVDRLIEKFSDP | 202 | Cpb2 |
| | | | | | TAWPRMDS | | |
| NBX0357 | GRTASM | 103 | ITRSSIYT | 153 | AADSTMSGSSR | 203 | Cpb2 |
| | | | | | YSSDYAY | | |
| NBX0358 | GFTFSNSPM | 104 | IDIGGNR | 154 | AKGLASTI | 204 | Cpb2 |
| NBX0359 | GRRFTLYHM | 105 | ISWSGGST | 155 | AVDRLIESFSDP | 205 | Cpb2 |
| | | | | | TAWPRMDY | | |
| NBX0360 | GRRFSLYHM | 106 | ISWSGGTT | 156 | AVDRLIESFSDP | 206 | Cpb2 |
| | | | | | TAWPRMDY | | |
| NBX0363 | ASIISIRVM | 341 | MSRGGTI | 459 | AALLDSYY | 577 | NetB |
| NBX0364 | GSIMSIRVM | 342 | MSRGGTI | 460 | TALLDSYY | 578 | NetB |
| NBX0365 | GSIFSIRVM | 343 | MSRGGTI | 461 | TALLDSYY | 579 | NetB |
| NBX0369 | ASIFSIRVM | 344 | MSRGNTI | 462 | AALLDSYY | 580 | NetB |
| NBX0370 | GSIMSIRVM | 345 | MSRGGTI | 463 | AALLDSYY | 581 | NetB |
| NBX0373 | ASIMSIRVM | 346 | MSRGNTI | 464 | AALLDSYY | 582 | NetB |
| NBX0374 | ASIMSIRVM | 347 | MSRGGTI | 465 | AALLDSYY | 583 | NetB |
| NBX0379 | GSIMSIRVM | 348 | MSRGNTI | 466 | AALLDSYY | 584 | NetB |
| NBX0380 | GSIISIRVM | 349 | MSRGGTI | 467 | AALLDSYY | 585 | NetB |
| NBX0381 | ASIISIRVM | 350 | MSRGNTI | 468 | AALLDSYY | 586 | NetB |
| NBX0501 | GSIFSINVM | 351 | ITSGGST | 469 | NAAQSRTSWLF | 587 | NetB |
| | | | | | PDEYDY | | |
| NBX0502 | GRTFSIYAM | 352 | INRGGGTT | 470 | AADRVTDTYYYL | 588 | NetB |
| | | | | | NPESYDY | | |
| NBX0503 | GSGRRVGYM | 353 | ISRAGAT | 471 | FASLIDAGTY | 589 | NetB |
| NBX0504 | GRTFSIYAM | 354 | INRSGGTT | 472 | AADRVTDTYYYL | 590 | NetB |
| | | | | | NPESYDY | | |
| NBX0505 | GMSFSLGTI | 355 | ITNADTT | 473 | NTATS | 591 | NetB |
| NBX0506 | GSGRRVGYM | 356 | ISRAGAT | 474 | FASVFDAGTY | 592 | NetB |
| NBX0507 | GSGRRVGYM | 357 | ISRAGAT | 475 | FASIFDAGTY | 593 | NetB |
| NBX0508 | GSGSRINYM | 358 | INRTGAA | 476 | FASYLGAGAY | 594 | NetB |
| NBX0509 | GRTFSTYTV | 359 | ITWNGGTI | 477 | VMGAAGQGWRY | 595 | NetB |
| NBX0510 | GSGSRINYM | 360 | INRTGAA | 478 | WASYLGAGTY | 596 | NetB |
| NBX0511 | GFTFSRNYM | 361 | IYSDDST | 479 | SKEGGL | 597 | NetB |
| NBX0512 | GSGRRVGYM | 362 | ISRAGAT | 480 | FASVFDAGTY | 598 | NetB |
| NBX0513 | GSGRRVGYM | 363 | ISRAGAT | 481 | FASLFDAGTY | 599 | NetB |
| NBX0514 | GRTFSGRTM | 364 | ITWSGGTT | 482 | ASDGPWRATTP | 600 | CnaA |
| | | | | | DAYDY | | |
| NBX0515 | GSIGTIDSM | 365 | IMFSGRT | 483 | YSNQY | 601 | CnaA |
| NBX0517 | EFSLLFGTI | 366 | VSSSDGST | 484 | ATRCTVVPGIT | 602 | CnaA |
| NBX0518 | GSITRVGGM | 367 | INEVGNT | 485 | WIPPIP | 603 | CnaA |
| NBX0519 | PFSLRLGVV | 368 | ISSSEGST | 486 | ATRCTVVPGIT | 604 | CnaA |
| NBX0520 | ARTSSSRAM | 369 | ISWSGGRT | 487 | AARRSDFTGDY | 605 | CnaA |
| | | | | | AYSGRSAYDY | | |
| NBX0521 | GSTFIFDKM | 370 | LMSRGDP | 488 | RGRAGERVY | 606 | CnaA |
| NBX0522 | GRTFSGVIV | 371 | TLWSGGST | 489 | AAKYGGSLSYM | 607 | CnaA |
| | | | | | HPTGYTY | | |
| NBX0523 | RIVFTISTM | 372 | INRSGALT | 490 | AASKANMPALP | 608 | CnaA |
| | | | | | ANYDY | | |
| NBX0524 | GSITRLGSM | 373 | ITAVGNT | 491 | WIPPIP | 609 | CnaA |
| NBX0525 | GSITRLGGM | 374 | IDTVGNT | 492 | WIPPIP | 610 | CnaA |
| NBX0526 | ERAFMYNM | 375 | RNWNVERT | 493 | ATTRVWPTQH | 611 | CnaA |
| | | | | | QMGQIEY | | |
| NBX0527 | SSFNTM | 376 | ITSGGTI | 494 | VADWQYGSTWNY | 612 | CnaA |
| NBX0528 | GRNFDYYSM | 377 | INWRGAVI | 495 | AAASSSSRLLEPI | 613 | CnaA |
| | | | | | GYNY | | |
| NBX0529 | GSMFSINDM | 378 | ISSGGTT | 496 | AGNLKRSETSYYWK | 614 | CnaA |
| NBX0530 | GRTFSRYHM | 379 | ISLSGGGT | 497 | TADRHEWGRL | 615 | CnaA |
| | | | | | MKGDY | | |
| NBX0531 | GRTSNSYNM | 380 | ISWTGGFT | 498 | AATSRSLTSAM | 616 | CnaA |
| | | | | | TREIRAYDY | | |
| NBX0532 | GSTFSFNKM | 381 | FMNDGNT | 499 | RGRAGMEVY | 617 | CnaA |
| NBX0533 | PLTLRLGPI | 382 | ISSRDDK | 500 | ATRCTVVPGIS | 618 | CnaA |
| NBX0534 | GRNFGYYTM | 383 | ITWRGVI | 501 | AAASSSSRPLEPI | 619 | CnaA |
| | | | | | GYNY | | |
| NBX0535 | GDIFSAAGM | 384 | VTWDGGTT | 502 | AAGNTGPFNLL | 620 | CnaA |
| | | | | | HSSAQYAY | | |
| NBX0536 | PLTLRLGAI | 385 | ITSTEDK | 503 | ATRCTVVPGIS | 621 | CnaA |
| NBX0537 | GSITRIGGM | 386 | INTVGNT | 504 | WIPPLP | 622 | CnaA |
| NBX0538 | GRSFSRYIM | 387 | IAPSGGSA | 505 | AARYDMDYEYKT | 623 | NetB |
| NBX0539 | GSGSRIGFM | 388 | INRTGAT | 506 | FASVVDAGTY | 624 | NetB |
| NBX0540 | GLTFSDYAM | 389 | ISLTAAST | 507 | AAQGRILRGRG | 625 | NetB |
| | | | | | LFKASDYDY | | |
| NBX0541 | GTISIFDPM | 390 | ISEGST | 508 | RLSRYYNSNIY | 626 | NetB |
| NBX0542 | RNIYGINVI | 391 | SANGGTT | 509 | KAELYTLQHNYEY | 627 | NetB |
| NBX0543 | GTLSLFDPM | 392 | ISGLST | 510 | HLSRYYNSNIY | 628 | NetB |
| NBX0544 | GRVLSINAM | 393 | ITNGGST | 511 | LAEERPYYGGPLEY | 629 | NetB |
| NBX0545 | RTTFRVGTM | 394 | ITSGGST | 512 | FANIVDRPVS | 630 | NetB |
| NBX0546 | GSGSRIGLM | 395 | IKGTGTT | 513 | FASVLGAGTY | 631 | NetB |
| NBX0547 | GTISLFDSM | 396 | ITEGST | 514 | RLSRYYNSNIY | 632 | NetB |
| NBX0548 | ETSLNFDDM | 397 | INTFPAGTTA | 515 | NAGDY | 633 | NetB |
| NBX0549 | GSDSSINYM | 398 | ISRDGRS | 516 | YVDPLGRVPR | 634 | NetB |
| NBX0550 | GTVNLM | 399 | IKGTGTT | 517 | FASVLGAGTY | 635 | NetB |
| NBX0551 | GSIFSRNII | 400 | INTGGRT | 518 | NAPSLGY | 636 | NetB |
| NBX0552 | GSGSINYM | 401 | INRTGAA | 519 | FASALGAGVY | 637 | NetB |
| NBX0553 | GSGWRVGYM | 402 | ISRAGAT | 520 | FASIIDAGTY | 638 | NetB |
| NBX0561 | GENFSTYVM | 403 | HNWRGGGT | 521 | AARSGGSYTYT | 639 | CnaA |
| | | | | | GSYHY | | |
| NBX0801 | GRTFSSYAM | 404 | ISRSGGST | 522 | AANRYGSSSYQ | 640 | CnaA |
| | | | | | GQYAS | | |
| NBX0802 | GRTFSSYHM | 405 | ISRSGGFT | 523 | AAQQWPDPRN | 641 | CnaA |
| | | | | | PNGYDY | | |
| NBX0803 | GRTFINYGM | 406 | VSISGAGT | 524 | AAAKAGHWGR | 642 | CnaA |
| | | | | | DANYDY | | |
| NBX0804 | GRTLTAYGM | 407 | VSLSGAST | 525 | AAAKAGQWGR | 643 | CnaA |
| | | | | | DAKYDY | | |
| NBX0805 | GRTFSTYAM | 408 | ISWSGGRI | 526 | TADLKGLWALG | 644 | CnaA |
| | | | | | LPGHYASWDS | | |
| NBX0806 | GSIGSINIM | 409 | FTSGGST | 527 | RARRGWAIY | 645 | CnaA |
| NBX0807 | GRTFSSYGM | 410 | ISRTGSGT | 528 | AADSGGSWGR | 646 | CnaA |
| | | | | | GTTYDY | | |
| NBX0808 | ARASSIGAM | 411 | VTAGADTT | 529 | AAYNTAGWGE | 647 | CnaA |
| | | | | | PHQSYRY | | |
| NBX0809 | GLTFGNYDM | 412 | ISSSGAYT | 530 | AGRRSVVVRSF | 648 | CnaA |
| | | | | | DYDY | | |
| NBX0810 | GRIFNANGM | 413 | LYRSGST | 531 | NVNWALHDS | 649 | CnaA |
| NBX0811 | ERTFSSDGM | 414 | ISRTGSAT | 532 | AANSGGHWW | 650 | CnaA |
| | | | | | RGATYDY | | |
| NBX0812 | GTIFSANGM | 415 | LYRDGST | 533 | NVNWALHDS | 651 | CnaA |
| NBX0847 | TRASIVGAM | 416 | IAAGSPSTP | 534 | AAYNTANWGQ | 652 | CnaA |
| | | | | | PHQSYRH | | |
| NBX0866 | GSILNINVM | 417 | IYRDGST | 535 | NVVTYGSNRRDF | 653 | CnaA |
| NBX0867 | GRTFSSYAM | 418 | ISRSGGST | 536 | AANRYGSSSYQ | 654 | CnaA |
| | | | | | GQYGS | | |
| NBX0868 | GRTFSSYAM | 419 | ISRSGGST | 537 | AANRYGSSSYQ | 655 | CnaA |
| | | | | | GQYDY | | |
| NBX0869 | GTIFSINGM | 420 | LYRGGST | 538 | NVNWALQDS | 656 | CnaA |
| NBX0870 | TSDGSINVM | 421 | ITSLGSQ | 539 | RARRGWAIY | 657 | CnaA |
| NBX0871 | GRTFNIYAM | 422 | ISDSGGSA | 540 | AADLTGLWALG | 658 | CnaA |
| | | | | | LPGHYASWDS | | |
| NBX0872 | GFTFRSSAM | 423 | IGSDGENI | 541 | QLGRTVLDYF | 659 | CnaA |
| NBX0873 | GRTFINYGM | 424 | VSSSGAGT | 542 | AAAKAGQWGR | 660 | CnaA |
| | | | | | YANYDY | | |
| NBX0874 | GRTFSSYAM | 425 | ISRSGGTT | 543 | AANPYGSSSYQ | 661 | CnaA |
| | | | | | GQYGS | | |
| NBX0875 | GRAFSGYAM | 426 | ISRGGGTT | 544 | AANRYGSSSYQ | 662 | CnaA |
| | | | | | GQYGS | | |
| NBX0876 | GRTFINYGM | 427 | VSSSGAGT | 545 | AAAKAGHWGR | 663 | CnaA |
| | | | | | DANYDY | | |
| NBX0877 | GSISSITFM | 428 | IARSGTT | 546 | YVDRRGAVPT | 664 | NetB |
| NBX0878 | GSISSITFM | 429 | IARSGTT | 547 | YVDRRDVVPT | 665 | NetB |
| NBX0879 | GTGFPIITFM | 430 | ISRGGVA | 548 | YADRFSGSPT | 666 | NetB |
| NBX0880 | VSSIGTM | 431 | ISRVGTT | 549 | FANVISGPVY | 667 | NetB |
| NBX0881 | TRFFSNYAM | 432 | ISRDGAVP | 550 | AASRQGNPYAQ | 668 | NetB |
| | | | | | TSYDY | | |
| NBX0883 | GSADSIKIM | 433 | ITSGGTT | 551 | NALVSRRDSAAYFA | 669 | NetB |
| NBX0884 | ESIVSITPM | 434 | TTRDGAP | 552 | KARKDSHDY | 670 | NetB |
| NBX0885 | ETIGSIQRM | 435 | RTNGGTT | 553 | NAHIREYYSTYDY | 671 | NetB |
| NBX0886 | GSISRIRDM | 436 | ISSGGST | 554 | NALFNPIDGPARYY | 672 | NetB |
| NBX0887 | GSISRIYDM | 437 | ISRGGST | 555 | TALFNPVDGTARYY | 673 | NetB |
| NBX0888 | GTIFSINVM | 438 | ITSGGQI | 556 | NAASSTWPPRDYDY | 674 | NetB |
| NBX0889 | RSISSIAAM | 439 | ITNGGST | 557 | NADERPYYGDSVLS | 675 | NetB |
| NBX0890 | GTGFPIITFM | 440 | INRGGVA | 558 | YADRFSGSPT | 676 | NetB |
| NBX0891 | GRTFSNYHM | 441 | ISRGTSTT | 559 | AADADRSTTIYS | 677 | NetB |
| | | | | | RDIYDY | | |
| NBX0892 | EGTFSNYRM | 442 | ISRDGAVP | 560 | AASRQGLPYVE | 678 | NetB |
| | | | | | TSYDY | | |
| NBX0893 | GSISSITFM | 443 | SARRGTT | 561 | YVDRRDEVPT | 679 | NetB |
| NBX0894 | GGTFSSYVM | 444 | IRWSRGST | 562 | AADGNPAKLVL | 680 | NetB |
| | | | | | DQYGMDY | | |
| NBX0895 | GSISEITYM | 445 | IARVGTT | 563 | YVDQRGVVPT | 681 | NetB |
| NBX0896 | ARASSIGAM | 446 | VNAGADTT | 564 | AAYNTAGWGE | 682 | CnaA |
| | | | | | PHQSYRY | | |
| NBX0897 | GSIFIISTM | 447 | ITSGGST | 565 | NAEVHVWGVP | 683 | CnaA |
| | | | | | GPRDY | | |
| NBX0898 | GRTFSSYAM | 448 | ISRSGGST | 566 | AANPYGSSSYQ | 684 | CnaA |
| | | | | | GQYAS | | |
| NBX0899 | GSIFSSNGM | 449 | LYRSGST | 567 | NVNWALHDS | 685 | CnaA |
| NBX08100 | GRTFSAYGM | 450 | VSGGGGGT | 568 | AAATAGHWGR | 686 | CnaA |
| | | | | | DANYDY | | |
| NBX08101 | GSIFSSNGM | 451 | LFRSGST | 569 | NVNWALHDS | 687 | CnaA |
| NBX08102 | GRTFSSYAM | 452 | ISRSGGTT | 570 | AANPYGSSSYQ | 688 | CnaA |
| | | | | | GQYGS | | |
| NBX08103 | GIIHSINVM | 453 | ISSGGRT | 571 | TMVWGLRYY | 689 | CnaA |
| NBX08104 | TSIFSSNGM | 454 | LFRSGST | 572 | NVNWALHDS | 690 | CnaA |
| NBX08105 | GRTFSSYAM | 455 | ISRGGGTT | 573 | AANPYGSSSYQ | 691 | CnaA |
| | | | | | GQYGS | | |
| NBX08106 | GSIFSSNGM | 456 | LYRSGST | 574 | NVNWALHDS | 692 | CnaA |
| NBX08107 | GSIFSSNGM | 457 | LYRSGST | 575 | NVNWALHDS | 693 | CnaA |
| NBX08108 | RSILSANGM | 458 | LYRSGST | 576 | NVNWALHDS | 694 | CnaA |

### Antibodies Recombinantly Expressed

In another aspect, the present invention provides a method for producing V_{H}H in a suitable producing organism. Suitable producing organisms include, without limitation, bacteria, yeast and algae. In certain embodiments, the producing bacterium is Escherichia coli. In certain embodiments, the producing bacterium is a member of the Bacillus genus. In certain embodiments, the producing bacterium is a probiotic. In certain embodiments, the yeast is Pichia pastoris. In certain embodiments, the yeast is Saccharomyces cerevisiae. In certain embodiments, the alga is a member of the Chlamydomonas or Phaeodactylum genera.

### Antibodies Added to Feed

In yet another aspect, the present invention provides a polypeptide or pluralities thereof comprising a V_{H}H or V_{H}Hs that bind disease-causing agents and are administered to host animals via any suitable route as part of a feed product. In certain embodiments, the animal is selected from the list of host animals described, with that list being representative but not limiting. In certain embodiments, the route of administration to a recipient animal can be, but is not limited to: introduction to the alimentary canal orally or rectally, provided to the exterior surface (for example, as a spray or submersion), provided to the medium in which the animal dwells (including air based media), provided by injection, provided intravenously, provided via the respiratory system, provided via diffusion, provided via absorption by the endothelium or epithelium, or provided via a secondary organism such as a yeast, bacterium, algae, bacteriophages, plants and insects. In certain embodiments, the host is from the superorder Galloanserae. In certain embodiments, the host is a poultry animal. In certain embodiments, the poultry animal is a chicken, turkey, duck, quail, pigeon, squab or goose. In certain embodiments, the poultry animal is a chicken.

### Feed Product

In a further aspect, the present invention provides a polypeptide or pluralities thereof comprising a V_{H}H or V_{H}Hs that bind disease-causing agents and are administered to host animals in the form of a product. The form of the product is not limited, so long as it retains binding to the disease-causing agent in the desired form. In certain embodiments, the product is feed, pellet, nutritional supplement, premix, therapeutic, medicine, or feed additive, but is not limited to these forms.

### Feeding Dosage

In a further aspect, the present invention provides a polypeptide or pluralities thereof comprising a V_{H}H or V_{H}Hs that bind disease-causing agents and are administered to host animals as part of a product at any suitable dosage regime. In practice, the suitable dosage is the dosage at which the product offers any degree of protection against a disease-causing agent, and depends on the delivery method, delivery schedule, the environment of the recipient animal, the size of the recipient animal, the age of the recipient animal and the health condition of the recipient animal among other factors. In certain embodiments, V_{H}Hs are administered to recipient animals at a concentration in excess of 1 mg/kg of body weight. In certain embodiments, V_{H}Hs are administered to recipient animals at a concentration in excess of 5 mg/kg of body weight. In certain embodiments, V_{H}Hs are administered to recipient animals at a concentration in excess of 10 mg/kg of body weight. In certain embodiments, V_{H}Hs are administered to recipient animals at a concentration in excess of 50 mg/kg of body weight. In certain embodiments, V_{H}Hs are administered to recipient animals at a concentration in excess of 100 mg/kg of body weight. In certain embodiments, V_{H}Hs are administered to recipient animals at a concentration less than 1 mg/kg of body weight. In certain embodiments, V_{H}Hs are administered to recipient animals at a concentration less than 500 mg/kg of body weight. In certain embodiments, V_{H}Hs are administered to recipient animals at a concentration less than 100 mg/kg of body weight. In certain embodiments, V_{H}Hs are administered to recipient animal at a concentration less than 50 mg/kg of body weight. In certain embodiments, V_{H}Hs are administered to recipient animals at a concentration less than 10 mg/kg of body weight.

### Feeding Frequency

In a further aspect, the present invention provides a polypeptide or pluralities thereof comprising a V_{H}H or V_{H}Hs that bind disease-causing agents and are administered to host animals as part of a product at any suitable dosage frequency. In practice, the suitable dosage frequency is that at which the product offers any protection against a disease-causing agent, and depends on the delivery method, delivery schedule, the environment of the recipient animal, the size of the recipient animal, the age of the recipient animal and the health condition of the recipient animal, among other factors. In certain embodiments, the dosage frequency can be but is not limited to: constantly, at consistent specified frequencies under an hour, hourly, at specified frequencies throughout a 24-hour cycle, daily, at specified frequencies throughout a week, weekly, at specified frequencies throughout a month, monthly, at specified frequencies throughout a year, annually, and at any other specified frequency greater than 1 year.

### Feed Additives

In a further aspect, the present invention provides a polypeptide or pluralities thereof comprising a V_{H}H or V_{H}Hs that bind disease-causing agents and are administered to host animals as part of a product that also comprises other additives or coatings. In practice, the most suitable coating or additive depends on the method of delivery, the recipient animal, the environment of the recipient, the dietary requirements of the recipient animal, the frequency of delivery, the age of the recipient animal, the size of the recipient animal, the health condition of the recipient animal In certain embodiments, these additives and coatings can include but are not limited to the following list and mixtures thereof: a vitamin, an antibiotic, a hormone, an antimicrobial peptide, a steroid, a probiotic, a probiotic, a bacteriophage, chitin, chitosan, B-1,3-glucan, vegetable extracts, peptone, shrimp meal, krill, algae, B-cyclodextran, alginate, gum, tragacanth, pectin, gelatin, an additive spray, a toxin binder, a short chain fatty acid, a medium chain fatty acid, yeast, a yeast extract, sugar, a digestive enzyme, a digestive compound, an essential mineral, an essential salt, or fibre.

### Non-Feed Uses

In a further aspect, the present invention provides a polypeptide or pluralities thereof comprising a V_{H}H or V_{H}Hs that bind disease-causing agents, and can be used in a non-feed use, such as but not limited to: a diagnostic kit, an enzyme-linked immunosorbent assay (ELISA), a western blot assay, an immunofluorescence assay, or a fluorescence resonance energy transfer (FRET) assay, in its current form and/or as a polypeptide conjugated to another molecule. In certain embodiments, the conjugated molecule is can be but is not limited to: a fluorophore, a chemiluminescent substrate, an antimicrobial peptide, a nucleic acid or a lipid.

### Antigens

In a further aspect, the present invention provides a polypeptide or pluralities thereof comprising a V_{H}H or V_{H}Hs that bind disease-causing agents, including toxins, produced by a species of Clostridium. In certain embodiments, the species does not belong to the Clostridium genus but is capable of harbouring disease-causing agents shared by Clostridium species. In certain embodiments, the Clostridium species refers to both current and reclassified organisms. In certain embodiments, the Clostridium species is Clostridium perfringens.

In certain embodiments, the V_{H}H or plurality thereof is capable of binding to one or more disease-causing agents, originating from the same or different species. In certain embodiments, the disease-causing agent is a polypeptide with 80% or greater amino acid sequence identity to NetB (SEQ ID 207). In certain embodiments, the disease-causing agent is a polypeptide with 80% or greater amino acid sequence identity to Cpa (SEQ ID 208). In certain embodiments, the disease-causing agent is a polypeptide with 80% or greater amino acid sequence identity to Cpb2 (SEQ ID 209). In certain embodiments, the disease-causing agent is a polypeptide with 80% or greater amino acid sequence identity to CnaA (SEQ ID 210). In certain embodiments, the disease-causing agent is a polypeptide with 80% or greater amino acid sequence identity to the collagen-binding domain of CnaA (SEQ ID 211). In certain embodiments, the disease-causing agent is an exposed peptide, protein, protein complex, nucleic acid, lipid, or combination thereof, that is associated to the surface of the Clostridium bacterium. In certain embodiments, the disease-causing agent is a pilus, fimbria, flagellum, secretion system or porin. In certain embodiments, the disease-causing agent is the Clostridium bacterium.

In certain embodiments, the disease-causing agent or a derivative thereof can be provided in excess and outcompete the activity of the pathogen expressed disease-causing agent. In certain embodiments, a polypeptide with 80% or greater amino acid sequence identity to CnaA (SEQ ID 210) or the collagen-binding domain of CnaA (SEQ ID 211) can be provided in excess to outcompete the activity of CnaA expressed by the Clostridium perfringens bacterium.

Antigen Sequences
NetB
>ABW71134.1 necrotic enteritis toxin B precursor
[Clostridium perfringens]
(SEQ ID NO: 207)
MKRLKIISITLVLTSVISTSLFSTQTQVFASELNDINKIELKNLSGEIIK
ENGKEAIKYTSSDTASHKGWKATLSGTFIEDPHSDKKTALLNLEGFIPSD
KQIFGSKYYGKMKWPETYRINVKSADVNNNIKIANSIPKNTIDKKDVSNS
IGYSIGGNISVEGKTAGAGINASYNVQNTISYEQPDFRTIQRKDDANLAS
WDIKFVETKDGYNIDSYHAIYGNQLFMKSRLYNNGDKNFTDDRDLSTLIS
GGFSPNMALALTAPKNAKESVIIVEYQRFDNDYILNWETTQWRGTNKLSS
TSEYNEFMFKINWQDHKIEYYL
Cpa
>WP_057230321.1 phospholipase [Clostridium
perfringens]
(SEQ ID NO: 208)
MKRKICKALICAALATSLWAGASTKVYAWDGKIDGTGTHAMIVTQGVSIL
ENDLSKNEPESVRKNLEILKENMHELQLGSTYPDYDKNAYDLYQDHFWDP
DTDNNFSKDNSWYLAYSIPDTGESQIRKFSALARYEWQRGNYKQATFYLG
EAMHYFGDIDTPYHPANVTAVDSAGHVKFETFAEERKEQYKINTAGCKTN
EDFYADILKNKDFNAWSKEYARGFAKTGKSIYYSHASMSHSWDDWDYAAK
VTLANSQKGTAGYIYRFLHDVSEGNDPSVGKNVKELVAYISTSGEKDAGT
DDYMYFGIKTKDGKTQEWEMDNPGNDFMTGSKDTYTFKLKDENLKIDDIQ
NMWIRKRKYTAFPDAYKPENIKIIANGKVVVDKDINEWISGNSTYNIK
Cpb2
>AEP94971.1 Beta2-toxin (plasmid) [Clostridium
perfringens]
(SEQ ID NO: 209)
MKKLIVKSTMMLLFSCLLCLGIQLPNTVKANEVNKYQSVMVQYLEAFKNY
DIDTIVDISKDSRTVTKEEYKNMLMEFKYDPNQKLKSYEITGSRKIDNGE
IFSVKTEFLNGAIYNMEFTVSYIDNKLMVSNMNRISIVNEGKCIPTPSFR
TQVCTWDDELSQYIGDAVSFTRSSKFQYSSNTITLNFRQYATSGSRSLKV
KYSVVDHWMWGDDIRASQWVYGENPDYARQIKLYLGSGETFKNYRIKVEN
YTPASIKVFGEGYCY
CnaA
>ALJ54440.1 putative collagen adhesin [Clostridium
perfringens]
(SEQ ID NO: 210)
MKINKKIFSMLFMVIVLFTCISSNFSVSASSIQRGRDISNEVVTSLVATP
NSINDGGNVQVRLEFKENHQRNIQSGDTITVKWTNSGEVFFEGYEKTIPL
YIKDQNVGQAVIEKTGATLTFNDKIDKLDDVGGWATFTLQGRNITSGNHE
HTGIAYIISGSKRADVNITKPESGTTSVFYYKTGSMYTNDTNHVNWWLLV
NPSKVYSEKNVYIQDEIQGGQTLEPDSFEIVVTWYDGYVEKFKGKEAIRE
FHNKYPNSNISVSENKITVNISQEDSTQKFINIFYKTKITNPKQKEFVNN
TKAWFKEYNKPAVNGESFNHSVQNINADAGVNGTVKGELKIIKTLKDKSI
PIKDVQFKMRRVDNTVIKDGKKELLLTTDDKGIANVKGLPVGKYEVKEIS
APEWIAFNPLIAPKLEFTISDQDTEGKLWAVENELKTISIPVEKVWVGQT
SERAEIKLFADGIEVDKVILNADNNWKHTFENKPEYNSETKQKINYSVSE
TTISGYESNITGDAKNGFIVTNTELPDLTIGKEVIGELGDKTKVFNFELT
LKQADGKPINGKFNYIGSVDDRYKKESIKPSDGEITFIEGKATITLSHGQ
EITIKDLPYGVTYKVMEKEANENGYLTTYNGNNEVTTGELKQDTKVQVVN
NKEFVPTTGISTTTEQGTMVGMVIFSIGILMVMIVVLLQLNKGLKR
CnaA Collagen Binding Domain
(SEQ ID NO: 211)
GRDISNEVVTSLVATPNSINDGGNVQVRLEFKENHQRNIQSGDTITVKWT
NSGEVFFEGYEKTIPLYIKDQNVGQAVIEKTGATLTFNDKIDKLDDVGGW
ATFTLQGRNITSGNHEHTGIAYIISGSKRADVNITKPESGTTSVFYYKTG
SMYTNDTNHVNWWLLVNPSKVYSEKNVYIQDEIQGGQTLEPDSFEIVVTW
YDGYVEKFKGKEAIREFHNKYPNSNISVSENKITVNISQEDSTQKFINIF
YKTKITNPKQKEFVNNTKAWFKEYNKPAVNGESFNHSVQNINADAGVNGT
VK

### Examples

The following illustrative examples are representative of the embodiments of the applications, systems and methods described herein and are not meant to be limiting in any way.

While preferred embodiments of the present invention are shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

### 1. Production of Antigens

Recombinant antigens can be purified from an E. coli expression system. For example, an antigen can be expressed at 18° C. in E. coli BL21 (DE3) cells grown overnight in autoinducing media (Formedium). Cells are then lysed by sonication in buffer A (250 mM NaCl, 50 mM CaCl₂), 20 mM Imidazole and 10 mM HEPES, pH 7.4) with 12.5 μg/ml DNase I, and 1× Protease inhibitor cocktail (Bioshop). The lysate is cleared by centrifugation at 22000×g for 30 minutes at 4° C., applied to a 5 ml HisTrap HP column (GE Healthcare) pre-equilibrated with buffer A, washed with ten column volumes of buffer A and eluted with a gradient of 0% to 60% (vol/vol) buffer B (250 mM NaCl, 50 mM CaCl₂), 500 mM imidazole and 10 mM HEPES, pH 7.4). The protein is then dialyzed overnight in the presence of TEV against buffer C (250 mM NaCl, 10 mM HEPES, pH 7.4 and 5 mM β-mercaptoethanol) at 4° C. The dialyzed protein is applied to a HisTrap HP column (GE Biosciences) pre-equilibrated with buffer C. 6×His-tagged TEV (“6×His” disclosed as SEQ ID NO: 695) and 6×His-tag (SEQ ID NO: 695) are bound to the column and the antigen is collected in the flowthrough. The sample is dialyzed overnight against buffer D (5 mM NaCl and 10 mM Tris pH 8.8) and then applied to a 5 ml HiTrap Q HP column (GE Healthcare). The protein is eluted with a gradient of 0% to 50% (vol/vol) buffer E (1.0 M NaCl and 10 mM Tris pH 8.8). Lastly, the eluate is loaded onto a Superdex 75 Increase 10/300 GL gel filtration column (GE Healthcare) using buffer F (400 mM NaCl and 20 mM HEPES pH 7.4). The protein sample is then concentrated to 1 mg/mL using Amicon concentrators with appropriate molecular weight cut-off (MWCO; Millipore). The purified protein is stored at −80° C.

### 2. Production of NBXs and Panning

### Llama Immunisation

A single llama is immunized with purified disease-causing agents, such as the antigens listed, which may be accompanied by adjuvants. The llama immunization is performed using 100 μg of each antigen that are pooled and injected for a total of four injections. At the time of injection, the antigens are thawed, and the volume increased to 1 ml with PBS. The 1 ml antigen-PBS mixture is then mixed with 1 ml of Complete Freund's adjuvant (CFA) or Incomplete Freund's adjuvant (IFA) for a total of 2 ml. A total of 2 ml is immunized per injection. Whole llama blood and sera are then collected from the immunized animal on days 0, 28, 49, 70. Sera from days 28, 49 and 70 are then fractionated to separate V_{H}H from conventional antibodies. ELISA can be used to measure reactivity against target antigens in polyclonal and V_{H}H-enriched fractions. Lymphocytes are collected from sera taken at days 28, 49, and 70.

### Panning

RNA isolated from purified llama lymphocytes is used to generate cDNA for cloning into phagemids. The resulting phagemids are used to transform E. coli TG-1 cells to generate a library of expressed V_{H}H genes. The phagemid library size can be ˜2.5×10⁷ total transformants and the estimated number of phagemid containing V_{H}H inserts can be estimated to be ˜100%. High affinity antibodies are then selected by panning against the antigens used for llama immunization. Two rounds of panning are performed and antigen-binding clones arising from round 2 are identified using phage ELISA. Antigen-binding clones are sequenced, grouped according to their CDR regions, and prioritized for soluble expression in E. coli and antibody purification.

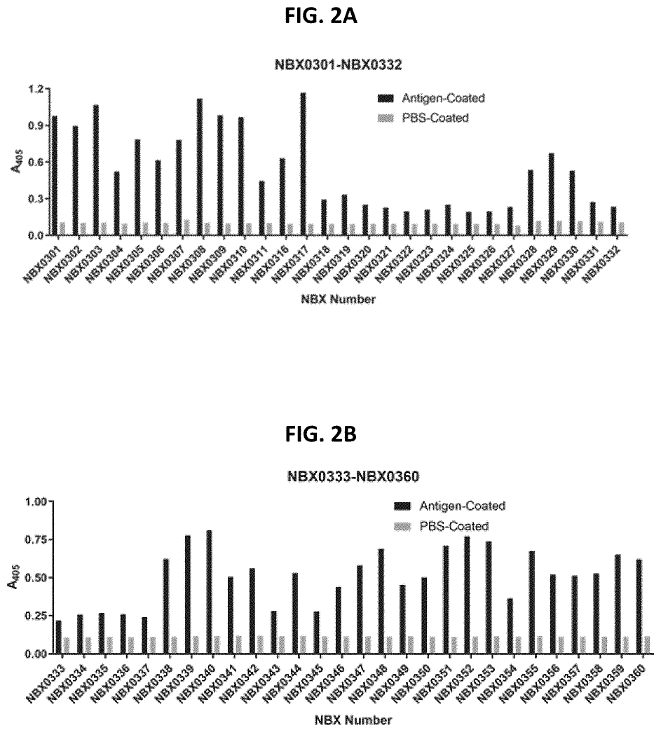

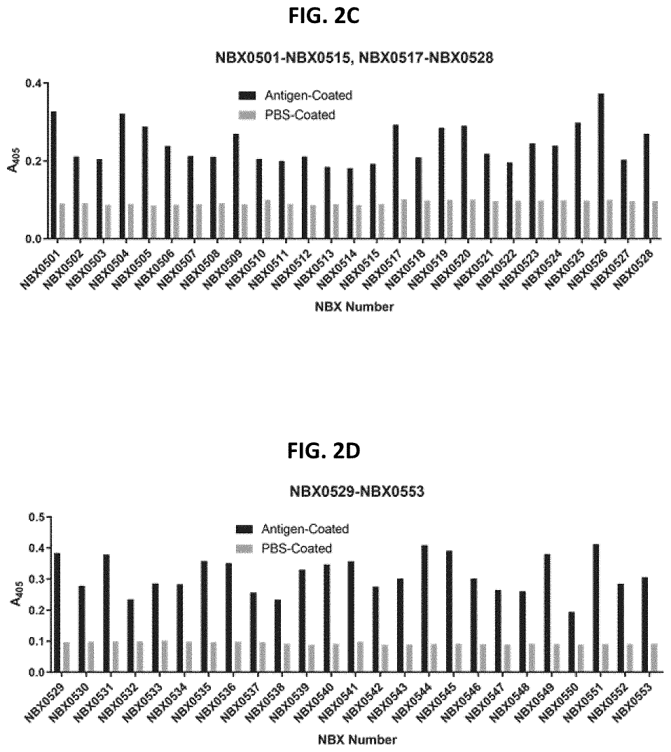

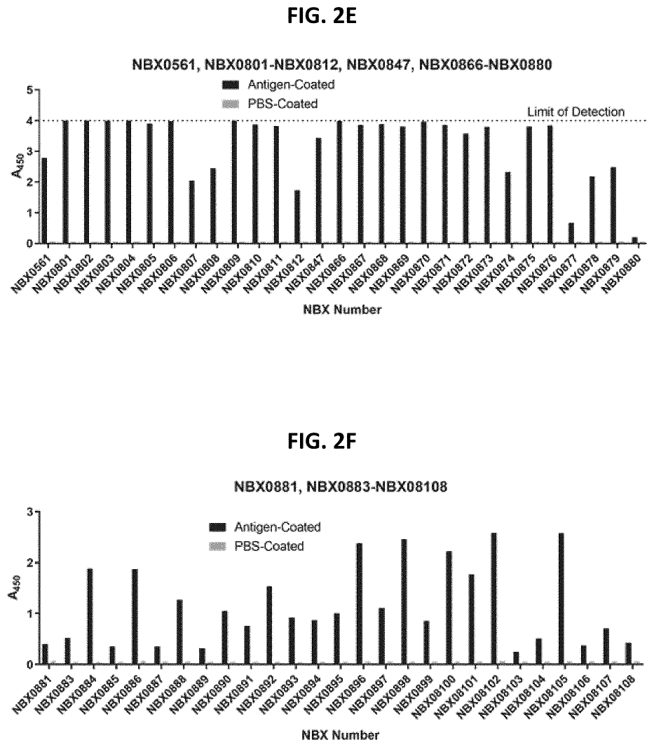

FIG. 2 shows the phage ELISA results for antibodies of this disclosure. Black bars show binding to wells coated with the antigen specified in Tables 1 and 2 dissolved in phosphate-buffered saline (PBS). Grey bars are negative controls that show binding to wells coated with PBS only. In all cases binding to the antigen target is at least twice above binding to the PBS-coated wells. Data for NBX0301 to NBX0332 are shown in panel A. Data for NBX0333-NBX0360 are shown in panel B. Data for NBX0501-NBX0515 and NBX0517-NBX0528 are shown in panel C. Data for NBX0529-NBX0553 are shown in panel D. Data for NBX0561, NBX0801-NBX0812, NBX0847, and NBX0866-NBX0880 are shown in panel E. Data for NBX0881 and NBX0883-NBX08108 are shown in panel F.

Purification of V_{H}Hs from E. coli

TEV protease-cleavable, 6×His-thioredoxin-NBX fusion proteins (“6×His” disclosed as SEQ ID NO: 695) are expressed in the cytoplasm of E. coli grown in autoinducing media (Formedium) for 24 hours at 30° C. Bacteria are collected by centrifugation, resuspended in buffer A (10 mM HEPES, pH 7.5, 250 mM NaCl, 20 mM Imidazole) and lysed using sonication. Insoluble material is removed by centrifugation and the remaining soluble fraction is applied to a HisTrap column (GE Biosciences) pre-equilibrated with buffer A. The protein is eluted from the column using an FPLC with a linear gradient between buffer A and buffer B (10 mM HEPES, pH 7.5, 500 mM NaCl, 500 mM Imidazole). The eluted protein is dialyzed overnight in the presence of TEV protease to buffer C (10 mM HEPES, pH 7.5, 500 mM NaCl). The dialyzed protein is applied to a HisTrap column (GE Biosciences) pre-equilibrated with buffer C. 6×His-tagged TEV and 6×His-tagged thioredoxin (“6×His” disclosed as SEQ ID NO: 695) are bound to the column and highly purified NBX is collected in the flowthrough. NBX proteins are dialyzed overnight to PBS and concentrated to ˜10 mg/ml.

Pichia pastoris strain GS115 with constructs for the expression and secretion of 6×His-tagged V_{H}H (“6×His” disclosed as SEQ ID NO: 695) are grown for 5 days at 30° C. with daily induction of 0.5% (vol/vol) methanol. Yeast cells are removed by centrifugation and the NBX-containing supernatant is spiked with 10 mM imidazole. The supernatant is applied to a HisTrap column (GE Biosciences) pre-equilibrated with buffer A (10 mM HEPES, pH 7.5, 500 mM NaCl). The protein is eluted from the column using an FPLC with a linear gradient between buffer A and buffer B (10 mM HEPES, pH 7.5, 500 mM NaCl, 500 mM Imidazole). NBX proteins are dialyzed overnight to PBS and concentrated to ˜10 mg/ml.

### 3. NBX Neutralization of NetB Cytotoxicity

Hepatocellular carcinoma-derived epithelial cells (LMH cells) from Gallus gallus strain Leghorn are adhered to the surface of a tissue-culture treated and gelatin-coated 96-well microtitre plate at 64,000 cells/well overnight at 37° C. and 5% CO₂. Recombinantly expressed NetB is preincubated with NBX at a range of concentrations or the buffer in which the NBXs are dissolved (20 mM HEPES pH 7.4, 150 mM NaCl) for 15 minutes at 37° C. and 5% CO₂. After 15 minutes the toxin/NBX mixtures are added to triplicate wells of LMH cells. The final concentration of NetB is 5 nM. The final concentrations of NBXs are 1, 3, 9, 27, 81, 243, 729, and 2187 nM. LMH cells with toxin/NBX mixtures are incubated for 5 hours at 37° C. and 5% CO₂. Cytotoxicity induced by NetB is measured using the Pierce LDH Cytotoxicity Assay Kit (Thermo Scientific) following the manufacturer's instructions. NetB percent cytotoxicity in the presence of NBX is determined relative to NetB cytotoxicity in the absence of NBX. A non-linear fit of the inhibitor concentration versus response is determined using GraphPad Prism 8 which generates the 50% inhibitory concentration (IC₅₀) which approximates the NBX concentration required to block 50% of the cytotoxicity of 5 nM NetB.

Table 3 indicates, for all NBXs tested, whether the NBX can neutralize the activity of NetB against LMH cells with an IC₅₀-value less than 1 μM and/or less than 50 nM.

TABLE 3
Summary table for NBXs that neutralize NetB

| NBX Number | IC₅₀ < 1 μM | IC₅₀ < 50 nM |
|---|---|---|
| NBX0301 | Yes | No |
| NBX0303 | Yes | Yes |
| NBX0304 | No | No |
| NBX0305 | Yes | Yes |
| NBX0307 | Yes | Yes |
| NBX0308 | Yes | No |
| NBX0309 | Yes | Yes |
| NBX0310 | Yes | Yes |
| NBX0311 | Yes | No |
| NBX0318 | Yes | Yes |
| NBX0319 | Yes | Yes |
| NBX0322 | Yes | No |
| NBX0323 | Yes | No |
| NBX0324 | Yes | Yes |
| NBX0362 | Yes | No |
| NBX0364 | Yes | Yes |
| NBX0365 | Yes | Yes |
| NBX0366 | Yes | Yes |
| NBX0370 | Yes | No |
| NBX0371 | Yes | Yes |
| NBX0372 | Yes | No |
| NBX0373 | Yes | No |
| NBX0375 | Yes | Yes |
| NBX0376 | Yes | No |
| NBX0378 | Yes | No |
| NBX0379 | Yes | No |
| NBX0501 | Yes | Yes |
| NBX0502 | No | No |
| NBX0503 | Yes | Yes |
| NBX0504 | No | No |
| NBX0505 | Yes | Yes |
| NBX0506 | Yes | Yes |
| NBX0507 | Yes | Yes |
| NBX0508 | Yes | No |
| NBX0509 | No | No |
| NBX0510 | Yes | Yes |
| NBX0511 | Yes | Yes |
| NBX0512 | Yes | No |
| NBX0513 | Yes | No |
| NBX0538 | Yes | Yes |
| NBX0539 | Yes | Yes |
| NBX0540 | Yes | Yes |
| NBX0541 | Yes | No |
| NBX0542 | Yes | Yes |
| NBX0543 | Yes | No |
| NBX0544 | Yes | Yes |
| NBX0545 | Yes | Yes |
| NBX0546 | Yes | Yes |
| NBX0547 | Yes | No |
| NBX0548 | Yes | Yes |
| NBX0549 | Yes | Yes |
| NBX0550 | Yes | Yes |
| NBX0551 | Yes | Yes |
| NBX0552 | Yes | Yes |
| NBX0553 | Yes | Yes |

### 4. NBX Reduction of CnaA Collagen Binding

In a 96-well microtiter plate, 2 μg of collagen is incubated in 100 μl of PBS per well overnight at 4° C. The plate is washed with 200 μl of PBS and then blocked with 200 μl of 5% skim milk in PBS for 2 hours at 37° C. During the blocking step, 200 nM or 2 μM of individual NBXs are mixed with or without 100 nM of 6×-Histidine (SEQ ID NO: 695) and Maltose-binding-protein (MBP) tagged CnaA in PBS for 30 minutes at 37° C. The plate is washed with 200 μl of PBS three times, and 100 μl of NBXs or NBX/MBP-CnaA mixture is added to each well for a 2-hour incubation at 37° C. After washing with 200 μl of PBS three times, 100 μl of 0.125 μg/ml of anti-His conjugated with HRP is added to each well and incubated for 1 hour at room temperature. The plate is then washed with 200 μl of PBS three times, and 100 μl of TMB substrate is added to each well and allowed to develop for 30 minutes. To stop the reaction, 50 μl of 1 M HCl is added to each well. Absorbance of the plate at 450 nm is read to quantify binding. To quantify the reduction of CnaA binding to collagen in the presence of NBX, a percent reduction is calculated relative to the binding of CnaA in the absence of NBX (100% binding).

Table 4 indicates, for all NBXs tested, whether the NBX can reduce binding of CnaA to collagen by more than 50% when the NBX is supplied at 2 μM and/or at 200 nM.

TABLE 4
Summary table for NBXs that neutralize CnaA

| | Collagen-binding | Collagen-binding |
|---|---|---|
| NBX | reduced by >50% | reduced by >50% |
| Number | at 2 μM | at 200 nM |
| NBX0316 | Yes | Yes |
| NBX0317 | Yes | Yes |
| NBX0325 | Yes | Yes |
| NBX0326 | Yes | Yes |
| NBX0327 | No | No |
| NBX0514 | No | No |
| NBX0515 | No | No |
| NBX0518 | No | No |
| NBX0520 | Yes | No |
| NBX0521 | No | No |
| NBX0522 | Yes | Yes |
| NBX0523 | No | No |
| NBX0524 | No | No |
| NBX0526 | No | No |
| NBX0527 | No | No |
| NBX0528 | Yes | Yes |
| NBX0529 | No | No |
| NBX0530 | Yes | Yes |
| NBX0531 | Yes | Yes |
| NBX0532 | No | No |
| NBX0533 | No | No |
| NBX0534 | Yes | Yes |
| NBX0535 | Yes | Yes |
| NBX0537 | No | No |
| NBX0801 | Yes | No |
| NBX0802 | Yes | No |
| NBX0803 | Yes | Yes |
| NBX0804 | Yes | Yes |
| NBX0805 | No | No |
| NBX0806 | No | No |
| NBX0807 | Yes | Yes |
| NBX0808 | Yes | No |
| NBX0809 | Yes | Yes |
| NBX0811 | Yes | Yes |
| NBX0812 | Yes | Yes |
| NBX0847 | Yes | No |
| NBX0866 | Yes | Yes |
| NBX0867 | Yes | No |
| NBX0868 | Yes | No |
| NBX0869 | Yes | Yes |
| NBX0870 | No | No |
| NBX0871 | No | No |
| NBX0872 | Yes | No |
| NBX0873 | Yes | Yes |
| NBX0874 | Yes | Yes |
| NBX0875 | Yes | Yes |
| NBX0876 | Yes | Yes |
| NBX0896 | Yes | No |
| NBX0897 | Yes | No |
| NBX0898 | Yes | No |
| NBX0899 | Yes | Yes |
| NBX08100 | Yes | Yes |
| NBX08101 | Yes | Yes |
| NBX08102 | Yes | Yes |
| NBX08103 | Yes | No |
| NBX08104 | Yes | Yes |
| NBX08105 | Yes | Yes |
| NBX08106 | Yes | Yes |
| NBX08107 | Yes | Yes |
| NBX08108 | Yes | Yes |

### 5. NBX Neutralization of Cpa Lecithinase Activity

Cpa is mixed with NBX or PBS to achieve a final concentration of 100 nM (Cpa) and 1 uM (NBX) in a total store-bought, free-range eggs by separation from the white. The yolk is punctured carefully then 5 ml is removed and mixed thoroughly with 45 ml PBS to create a 10% solution. The solution is centrifuged at 500 g to remove large aggregates and then passed through a 0.45 um GD/X syringe filter. 60 ul of the filtered yolk solution is added to the Cpa or Cpa/NBX wells to achieve a final concentration of 5% v/v egg yolk. The plate is incubated for 1 hr at 37° C. after which the optical density of the plate is measured at 620 nm. NBX neutralization of Cpa lecithinase activity is determined relative to Cpa lecithinase activity in the absence of NBX (100%).

Table 5 indicates, for all NBXs tested, whether the NBX can reduce Cpa lecithinase activity by more than 40% when the NBX is supplied at 1 μM.

TABLE 5
Summary table for NBXs that neutralize Cpa

| | Cpa lecithinase activity |
|---|---|
| NBX Number | reduced by >40% at 1 μM |
| NBX0329 | Yes |
| NBX0330 | No |
| NBX0338 | Yes |
| NBX0339 | Yes |
| NBX0340 | No |
| NBX0341 | No |

### 6. Untagged CnaA Provided in Excess Outcompetes Tagged CnaA for Collagen Binding

In a 96-well microtiter plate, 2 μg of collagen is incubated in 100 μl of PBS per well overnight at 4° C. The plate is washed with 200 μl of PBS and then blocked with 200 μl of 5% skim milk in PBS for 2 hours at 37° C. During the blocking step, 100 nM of 6×-Histidine (SEQ ID NO: 695) and Maltose-binding-protein (MBP) tagged CnaA is mixed with between 0 and 2000 nM untagged CnaA in PBS for 30 minutes at 37° C. The plate is washed with 200 μl of PBS three times, and 100 μl of MBP-CnaA or MBP-CnaA/untagged CnaA mixture is added to each well for a 2-hour incubation at 37° C. After washing with 200 μl of PBS three times, 100 μl of 0.125 μg/ml of anti-His conjugated with HRP is added to each well and incubated for 1 hour at room temperature. The plate is then washed with 200 μl of PBS three times, and 100 μl of TMB substrate is added to each well and allowed to develop for 30 minutes. To stop the reaction, 50 μl of 1 M HCl is added to each well. Absorbance of the plate at 450 nm is read to quantify binding.

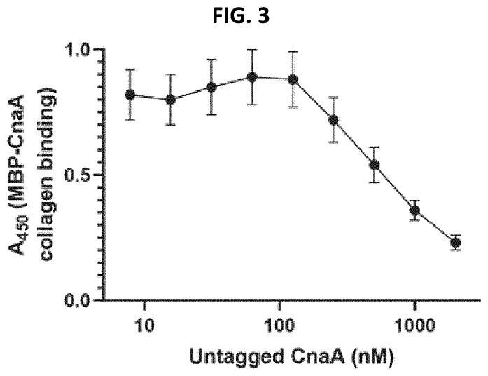

FIG. 3 shows the reduction of binding of MBP-CnaA to collagen in the presence of increasing concentrations of untagged CnaA.

All publications, patent applications, issued patents, and other documents referred to in this specification are herein incorporated by reference as if each individual publication, patent application, issued patent, or other document is specifically and individually indicated to be incorporated by reference in its entirety. Definitions that are contained in text incorporated by reference are excluded to the extent that they contradict definitions in this disclosure.

The following references are incorporated by reference in their entirety.
1. Wade, B. & Keyburn, A. (2015). The true cost of necrotic enteritis. World Poultry, 31, pp. 16-17
2. Moore, R. J. (2016). Necrotic enteritis predisposing factors in broiler chickens. Avian Pathology, 45(3), pp. 275-281.
3. Abid, S. A. et al. (2016). Emerging threat of necrotic enteritis in poultry and its control without use of antibiotics: a review. The Journal of Animal and Plant Sciences, 26(6), pp. 1556-1567.
4. Prescott, J. F. et al. (2011). The pathogenesis of necrotic enteritis in chickens: what we know and what we need to know: a review. Avian Pathology, 45(3), pp. 288-294.
5. Collier, C. T. et al. (2008) Coccidia-induced mucogenesis promotes the onset of necrotic enteritis by supporting Clostridium perfringens growth. Veterinary Immunology and Immunopathology, 122(1-2), pp. 104-115.
6. Van Meirhaeghe, H. & De Gussem, M. (2014). Coccidiosis a major threat to the chicken gut. Retrieved on May 25, 2018 from: https://www.poultryworld.net/Home/General/2014/9/Coccidiosis-a-major-threat-to-the-chicken-gut-1568808W/?dossier=35765&widgetid=1.
7. Chapman, H. D. (2014). Milestones in avian coccidiosis research: a review. Poultry Science, 93(3), pp. 501-511.
8. Shivaramaiah, S. et al. (2011). The role of an early Salmonella Typhimurium infection as a predisposing factor for necrotic enteritis in a laboratory challenge model. Avian Diseases, 55(2), pp. 319-323.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A polypeptide capable of: a) reducing the cytotoxicity of NetB against LMH cells with an IC50 value less than 1 mM; b) reducing the binding of nM CnaA to collagen by greater than 50% at 2 uM; or c) reducing Cpa lecithinase activity by greater than 40% at 1 uM.

2.-6. (canceled)

7. The polypeptide of claim 1, wherein the polypeptide comprises a plurality of VHHs.

8. The polypeptide of claim 7, wherein the polypeptide comprises at least three VHHs.

9. The polypeptide of claim 7, wherein any one of the plurality of VHHs is identical to another VHH of the plurality of VHHs.

10. The polypeptide of claim 7, wherein the plurality of VHHs are covalently coupled to one another by a linker, the linker comprising one or more amino acids.

11. The polypeptide of claim 1, wherein the polypeptide comprises a variable region fragment of a heavy chain antibody that comprises an amino acid sequence at least 80% identical to any one of SEQ ID Nos: 1 to 56 or 212 to 340.

12. The polypeptide of claim 1, wherein the polypeptide comprises a variable region fragment of a heavy chain antibody that comprises a complementarity determining region 1 (CDR1) as set forth in any one of SEQ ID Nos: 57 to 106 or 341 to 458, a complementarity determining region 2 (CDR2) as set forth in any one of SEQ ID Nos: 107 to 156 or 459 to 576, and a complementarity determining region 3 (CDR3) as set forth in any one of SEQ ID Nos: 157 to 206 or 577 to 694.

13.-15. (canceled)

16. The polypeptide of claim 1, wherein the species of Clostridium is Clostridium perfringens.

17. The polypeptide of claim 16, wherein the VHH specifically binds a Clostridium virulence factor.

18. The polypeptide of claim 16, wherein the VHH specifically binds an antigen or polypeptide at least 60% identical to SEQ IDs Nos: 207 to 211 or combinations thereof.

19.-39. (canceled)

40. A method of reducing or preventing a poultry-associated bacterial infection in a poultry animal, another animal species, or human individual comprising administering the polypeptide of claim 1 to the poultry animal, another animal species, or human individual.

41. A method of reducing transmission or preventing transmission of a poultry-associated bacterial from a poultry species to another poultry animal, another animal species, or a human individual comprising administering the polypeptide of claim 1 to the poultry species to another poultry animal, another animal species, or the human individual.

42. The method of claim 40, wherein the poultry animal is a species of a chicken, turkey, duck, quail, pigeon, squab, ostrich, or goose.

43. The method of claim 40, wherein the non-poultry animal species is a pig, sheep, goat, horse, cow, llama, alpaca, mink, rabbit, dog, cat, or human

44. The method of claim 40, wherein the polypeptide is adapted for introduction to the alimentary canal orally or rectally, provided to the exterior surface (for example, as a spray or submersion), provided to the medium in which the animal dwells (including air based media), provided by injection, provided intravenously, provided via the respiratory system, provided via diffusion, provided via absorption by the endothelium or epithelium, or provided via a secondary organism such as a yeast, bacterium, algae, bacteriophages, plants and insects to a host.

45. The method of claim 41, wherein the poultry animal is a species of a chicken, turkey, duck, quail, pigeon, squab, ostrich, or goose.

46. The method of claim 41, wherein the non-poultry animal species is a pig, sheep, goat, horse, cow, llama, alpaca, mink, rabbit, dog, cat, or human

47. The method of claim 41, wherein the polypeptide is adapted for introduction to the alimentary canal orally or rectally, provided to the exterior surface (for example, as a spray or submersion), provided to the medium in which the animal dwells (including air based media), provided by injection, provided intravenously, provided via the respiratory system, provided via diffusion, provided via absorption by the endothelium or epithelium, or provided via a secondary organism such as a yeast, bacterium, algae, bacteriophages, plants and insects to a host.
